(19) United States
(12) Patent Application Publication
(10) Pub. No.: US 2026/0125426 A1
(43) Pub. Date: May 7, 2026

(54) CYCLIC CELL-PENETRATING PEPTIDES AND USES THEREOF

(71) Applicant: Ohio State Innovation Foundation, Columbus, OH (US)

(72) Inventors: Dehua PEI, Columbus, OH (US); Jeremy RITCHEY, Columbus, OH (US)

(21) Appl. No.: 19/099,259

(22) PCT Filed: Jul. 31, 2023
(86) PCT No.: PCT/US2023/029093

§ 371 Date: Jan. 28, 2025

Related U.S. Application Data
(60) Provisional application No. 63/393,649, filed on Jul. 29, 2022.
(60) Provisional application No. 63/415,415, filed on Oct. 12, 2022.
(60) Provisional application No. 63/463,953, filed on May 4, 2023.

(51) Int. Cl. C07K 7/54; C07K 14/00

(52) U.S. Cl. CPC C07K 7/54; C07K 14/001; C07K 2319/10

(57) ABSTRACT

Disclosed are peptides comprising: a cell-penetrating peptide domain of from about 7 to about 25 amino acids in length; wherein the cell-penetrating peptide domain comprises any combination of at least two arginines and at least two amino acids having a hydrophobic side chain selected from an aryl or heteroaryl, wherein the aryl and heteroaryl are optionally substituted, and wherein the cell penetrating peptide domain comprises at least three thiol containing residues and which are separated from one another by at least one amino acids.

### CROSS REFERENCE TO RELATED APPLICATIONS

This application claims the benefit of priority to U.S. Provisional Application Nos. 63/393,649, filed Jul. 29, 2022, 63/415,415, filed Oct. 12, 2022, and 63/463,953, filed May 4, 2023, which are each incorporated by reference herein in their entireties.

### ACKNOWLEDGEMENT OF GOVERNMENT SUPPORT

This invention was made with government support under grant/contract number GM122459 awarded by the National Institutes of Health. The government has certain rights in the invention.

### BACKGROUND

Cell-penetrating peptides (CPPs) are short peptides (usually 5-30 aa) that are capable of entering the eukaryotic cell without causing significant damage to the cell membrane. As such, CPPs have potential applications in delivering membrane-impermeable cargoes (e.g., peptides, proteins, nucleic acids, and nanoparticles) into the interior of mammalian cells to act as novel therapeutics Since the initial discovery of Tat (Vivès, E. et al. (1997) Truncated HIV-1 Tat protein basic domain rapidly translocates through the plasma membrane and accumulates in the cell nucleus. J. Biol. Chem. 272, 16010-16017) and Penetratin (Derossi, D. et al. (1994) The third helix of the Antennapedia homeodomain translocates through biological membranes. J. Biol. Chem. 269, 10444-10450), nearly 2000 different CPPs have been reported. The first-generation CPPs are linear peptides, which generally have low cytosolic entry efficiencies and are also susceptible to proteolytic degradation in vivo. These shortcomings prompted us and other researchers to develop cyclic peptides as second-generation CPPs (Lättig-Tünnemann. G., et al. (2011) Backbone rigidity and static presentation of guanidinium groups increases cellular uptake of arginine-rich cell-penetrating peptides. Nat. Commun. 2, 453-459: Mandal, D., et al. (2011) Cell-penetrating homochiral cyclic peptides as nuclear-targeting molecular transporters. Angew. Chem. Int. Ed. 50 (41), 9633-9637; Qian, Z. et al. (2013) Efficient delivery of cyclic peptides into mammalian cells with short sequence motifs. ACS Chem. Biol. 8 (2). 423-431). Cyclic CPPs, as exemplified by cyclo(phe-Nal-Arg-arg-Arg-arg-Gln) (CPP9, where phe is D-phenylalanine, arg is D-arginine, and Nal is L-naphthylalanine) and cyclo(Phe-phe-Nal-Arg-arg-Arg-arg-Gln) (CPP12) (Qian, Z. et al. (2016) Discovery and mechanism of highly efficient cyclic cell-penetrating peptides. Biochemistry 55, 2601-2612), are highly resistant to proteolytic degradation and have vastly improved cytosolic entry efficiencies, compared to their linear counterparts. The latter properties in turn facilitated the mechanistic studies of how CPPs enter the eukaryotic cell. It is now established that CPPs bind to the proteoglycans and/or phospholipids on the cell membrane and are taken into the early endosome by endocytic/pinocytic mechanisms (Dougherty, P. G. et al. (2019) Understanding cell penetration of cyclic peptides. Chem. Rev. 119, 10241-10287). As the endosomes mature and become progressively more acidic, the CPPs bind to the endosomal membrane with increased affinity and cluster the phospholipids into CPP-enriched lipid domains (Qian. Id.: Sahni, A. et al. (2020) Cell-penetrating peptides escape the endosome by inducing vesicle budding and collapse. ACS Chem. Biol. 15, 2485-2492). The lipid domains bud out as small, unstable vesicles, which subsequently collapse, releasing their luminal contents into the cytosol (Id.). At high concentrations, CPPs can also induce the vesicle budding-and-collapse (VBC) mechanism at the plasma membrane, leading to direct translocation of the CPPs across the plasma membrane. Cyclization of CPPs increases their membrane-binding affinity and therefore the cell entry efficiency (Qian. Id.).

Cyclic CPPs have been used to deliver a variety of drug modalities into the mammalian cell in vitro and in vivo, including peptides (Qian, Z., et al. (2014) Early endosomal escape of a cyclic cell-penetrating peptide allows effective cytosolic cargo delivery. Biochemistry 53, 4034-4046: Dougherty, P. G., et al. (2020) A peptidyl inhibitor that blocks calcineurin-NFAT interaction and prevents acute lung injury. J. Med. Chem. 63 (21), 12853-12872: Dougherty. P. G., et al. (2019) Enhancing the cell permeability of stapled peptides with a cyclic cell-penetrating peptide. J. Med. Chem. 62 (22), 10098-10107: Salim, H., et al. (2020) Development of a cell-permeable cyclic peptidyl inhibitor against the Keap1-Nrf2 interaction. J. Org. Chem. 85(3), 1416-1424), proteins (Nischan, N., et al. (2015) Covalent attachment of cyclic TAT peptides to GFP results in protein delivery into live cells with immediate bioavailability. Angew. Chem. Int. Ed. 54(6), 1950-1953; Herce, H. D., et al. (2017) Cell-permeable nanobodies for targeted immunolabelling and antigen manipulation in living cells. Nature Chem. 9, 762-771; Schneider. A. F. L., et al. (2019) Targeted subcellular protein delivery using cleavable cyclic cell-penetrating peptides. Bioconjugate Chem. 30(2), 400-404: Zhang, W., et al. (2021) An intracellular nanobody targeting T4SS effector inhibits Ehrlichia infection. Proc. Natl. Acad. Sci. U.S.A. 118, e2024102118: Shintaku, J., et al. (2020) Thymidine phosphorylase intracellular enzyme replacement therapy in a murine model of mitochondrial neurogastrointestinal encephalopathy (MNGIE). Neurology 94, 3975), and nucleic acids (Soudah, T., et al. (2019) AntimiR-155 cyclic peptide-PNA conjugate: synthesis, cellular uptake, and biological activity. ACS Omega 4(9), 13954-13961: Cai, B., et al. (2019) Selection of DNA-encoded libraries to protein targets within and on Living Cells. J. Am. Chem. Soc. 141 (43), 17057-17061). However, backbone cyclized CPPs such as CPP9 and CPP12 must be chemically synthesized and subsequently conjugated to a cargo of interest. For protein cargoes, conjugation of a cyclic CPP to a specific site on the cargo protein remains a significant challenge in its own right; current conjugation methods often give a mixture of different products. Additionally, chemical conjugation of a CPP to a therapeutic protein significantly increases its cost of production. To overcome these limitations, genetically encodable CPPs, which can be fused to a peptide/protein cargo recombinantly, are being developed. A loop replacement strategy was developed in which a nonfunctional loop sequence of a cargo protein is replaced with a linear CPP motif (e.g., RRRRWWW, SEQ ID NO:1) (Chen, K., & Pei, D. (2020). Engineering Cell-Permeable Proteins through Insertion of Cell-Penetrating Motifs into Surface Loops. ACS Chemical Biology, 15(9), 2568-2576). Application of the strategy to the fibronectin type III domain led to a mammalian membrane transduction domain (MTD), which can be genetically fused to any peptide or protein cargo.

What are still needed, however, are new CPPs and strategies for making and using them. The compositions and methods disclosed herein address these and other needs.

### SUMMARY

Disclosed herein are compounds, compositions, methods for making and using such compounds and compositions. In various embodiments disclosed herein are cyclic peptides, compositions comprising such cyclic peptides, and methods of making and using them. In one aspect, disclosed are peptides comprising a cell-penetrating peptide domain of from about 7 to about 25 amino acids in length; wherein the cell-penetrating peptide domain comprises any combination of at least two arginines and at least two amino acids having a hydrophobic side chain selected from an aryl or heteroaryl, wherein the aryl and heteroaryl are optionally substituted, and wherein the cell penetrating peptide domain comprises at least three amino acids with a thiol containing side chain and which are separated from one another by at least two amino acids. Methods of making and using these peptides are also disclosed, as are derivatives and formulations thereof.

### BRIEF DESCRIPTION OF FIGURES

The accompanying figures, which are incorporated in and constitute a part of this specification, illustrate several aspects and together with the description serve to explain the principles of the invention.

FIGS. 1A and 1B are schemes showing the strategies for generating fusion proteins containing genetically encoded cyclic CPPs at their N- or C-terminus (FIG. 1A) or an internal loop region (FIG. 1B). Proteins containing linear CPP precursor sequences are produced recombinantly and then post-translationally converted into a bicyclic CPP by the addition of a Bi³⁺ ion.

FIGS. 2A and 2B show bismuth-mediated peptide cyclization. (FIG. 2A) Bismuth-mediated cyclization reaction for peptide 1 to form BCP1. (FIG. 2B) Analytical HPLC chromatogram of the crude cyclization reaction of BCP1.

FIG. 3 shows a series of live-cell confocal microscopic images of HeLa cells after treatment with 5 μM TMR-labeled peptides.

FIG. 4 shows the structures of naphthofluorescein (NF)-labeled representative BCPs and control peptides.

FIGS. 5A and 5B show cellular entry efficiency of BCPs as determined by flow cytometry. FIG. 5A is an overlay of raw flow cytometry data. FIG. 5B is a comparison of BCPs with Tat and CPP12. All values are relative to that of CPP12 (100%) and represent the mean±SD of three independent experiments.

FIG. 6 shows a live-cell confocal microscopy of HeLa cells after treatment with 2 μM TMR-labeled pBCP427-4 or CPP12 for 2 h at 37° C.

FIG. 7 shows live-cell confocal microscopy of HeLa cells after treatment with 2 or 5 μM TMR-labeled BCP427-1 or CPP12 for 2 h at 37° C. Note that at 5 μM. CPP12TMR resulted in direct translocation in many of the cells and intense diffuse fluorescence.

FIG. 8 shows serum stability of Tat, BCP4 (no Bi3+), BCP4, BCP16, and pBCP427-4. The amount of intact peptide remaining (relative to time 0) is plotted as a function of the incubation time.

FIGS. 9A and 9B show the effect of BCP16 and pBCP427-4 on the viability of HEK293T (9A) and HeLa cells (9B).

FIG. 10 shows structures of NF-labeled BCP16a, BCP16b, and pBCP427-4a.

FIGS. 11A and 11B show UPLC-MS analysis of BCP16a before (11A) or after incubation in human serum for 24 h (11B). The abundance of the [M+3H]³⁺ ion (at m/z 667) is plotted against the retention time.

FIG. 12 shows confocal microscopic images of NIH 3T3 cells after treatment with treatment with 2 μM peptide at 37° C. for 2 h.

FIG. 13A shows structures of CPP/BCP-K1 conjugates. FIG. 13B shows dose-dependent induction of luminescence signal in ARE-Reporter HepG2 cells by K1. CPP12-K1, BCP4-K1, BCP16-K1, and pBCP427-4-K1.

FIG. 14A shows structures of CPP/BCP-K1 conjugates. FIG. 14B shows dose-dependent induction of luminescence signal in ARE-Reporter HepG2 cells by K1, CPP12-K1, BCP16a-K1, and BCP16b-K1.

FIG. 15 shows confocal microscopic images of HeLa cells after treatment with 5 μM SEP or pBCP427-4-SEP for 4 h at 37° C. Top panels, fluorescence in the GFP channel; bottom panels, merged images of the GFP, DAPI, and DIC channels.

FIG. 16 shows serum stability of pBCP427-4 and pBCP427-4Ala. The amount of intact peptide remaining (relative to time 0) is plotted as a function of the incubation time.

### DETAILED DESCRIPTION

The present invention can be understood more readily by reference to the following detailed description of the invention and the Examples and Figures included therein.

Before the present compounds, compositions, articles, systems, devices, and/or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

### General Definitions

As used herein, nomenclature for compounds, including organic compounds, can be given using common names, IUPAC, IUBMB, or CAS recommendations for nomenclature. When one or more stereochemical features are present, Cahn-Ingold-Prelog rules for stereochemistry can be employed to designate stereochemical priority, E Z specification, and the like. One of skill in the art can readily ascertain the structure of a compound if given a name, either by systemic reduction of the compound structure using naming conventions, or by commercially available software, such as CHEMDRAW™ (Cambridgesoft Corporation, U.S.A.).

As used in the specification and the appended claims, the singular forms “a,” “an” and “the” include plural referents unless the context clearly dictates otherwise. Thus, for example, reference to “a functional group,” “an alkyl,” or “a residue” includes mixtures of two or more such functional groups, alkyls, or residues, and the like.

Ranges can be expressed herein as from “about” one particular value, and/or to “about” another particular value. When such a range is expressed, a further aspect includes from the one particular value and/or to the other particular value. Similarly, when values are expressed as approximations, by use of the antecedent “about,” it will be understood that the particular value forms a further aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

References in the specification and concluding claims to parts by weight of a particular element or component in a composition denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent (wt. %) of a component, unless specifically stated to the contrary, is based on the total weight of the formulation or composition in which the component is included.

As used herein, the terms “optional” or “optionally” means that the subsequently described event or circumstance can or cannot occur, and that the description includes instances where said event or circumstance occurs and instances where it does not.

As used herein, the term “subject” refers to the target of administration, e.g., a subject. Thus, the subject of the herein disclosed methods can be a vertebrate, such as a mammal, a fish, a bird, a reptile, or an amphibian. Alternatively, the subject of the herein disclosed methods can be a human, non-human primate, horse, pig, rabbit, dog, sheep, goat, cow, cat, guinea pig, fish, bird, or rodent. The term does not denote a particular age or sex. Thus, adult and new born subjects, as well as fetuses, whether male or female, are intended to be covered. In some examples, the subject is a mammal. A patient refers to a subject afflicted with a disease or disorder. The term “patient” includes human and veterinary subjects. In some examples of the disclosed methods, the subject has been diagnosed with a need for treatment of cancer prior to the administering step. In some examples of the disclosed method, the subject has been diagnosed with cancer prior to the administering step. The term subject also includes a cell, such as an animal, for example human, cell.

As used herein, the term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder: preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. In some examples, the term covers any treatment of a subject, including a mammal (e.g., a human), and includes: (i) preventing the disease from occurring in a subject that can be predisposed to the disease but has not yet been diagnosed as having it: (ii) inhibiting the disease, i.e., arresting its development: or (iii) relieving the disease, i.e., causing regression of the disease. In some examples, the subject is a mammal such as a primate, and, in some examples, the subject is a human. The term “subject” also includes domesticated animals (e.g., cats, dogs, etc.), livestock (e.g., cattle, horses, pigs, sheep, goats, fish, bird, etc.), and laboratory animals (e.g., mouse, rabbit, rat, guinea pig, fruit fly, etc.).

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

As used herein, the term “diagnosed” means having been subjected to a physical examination by a person of skill, for example, a physician, and found to have a condition that can be diagnosed or treated by the compounds, compositions, or methods disclosed herein. For example, “diagnosed with cancer” means having been subjected to a physical examination by a person of skill, for example, a physician, and found to have a condition that can be diagnosed or treated by a compound or composition that can treat or prevent cancer. As a further example, “diagnosed with a need for treating or preventing cancer” refers to having been subjected to a physical examination by a person of skill, for example, a physician, and found to have a condition characterized by cancer or other disease wherein treating or preventing cancer would be beneficial to the subject.

As used herein, the phrase “identified to be in need of treatment for a disorder.” or the like, refers to selection of a subject based upon need for treatment of the disorder. For example, a subject can be identified as having a need for treatment of a disorder (e.g., a disorder related to cancer) based upon an earlier diagnosis by a person of skill and thereafter subjected to treatment for the disorder. It is contemplated that the identification can. In some examples, be performed by a person different from the person making the diagnosis. It is also contemplated, in some examples, that the administration can be performed by one who subsequently performed the administration.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, and subcutaneous administration. Administration can be continuous or intermittent. In some examples, a preparation can be administered therapeutically: that is, administered to treat an existing disease or condition. In some examples, a preparation can be administered prophylactically: that is, administered for prevention of a disease or condition.

The term “contacting” as used herein refers to bringing a disclosed compound and a cell, target receptor, or other biological entity together in such a manner that the compound can affect the activity of the target (e.g., receptor, transcription factor, cell, etc.), either directly; i.e., by interacting with the target itself, or indirectly; i.e., by interacting with another molecule, co-factor, factor, or protein on which the activity of the target is dependent.

As used herein, the terms “effective amount” and “amount effective” refer to an amount that is sufficient to achieve the desired result or to have an effect on an undesired condition. For example, a “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In some examples, a preparation can be administered in a “prophylactically effective amount”; that is, an amount effective for prevention of a disease or condition.

As used herein, “EC₅₀,” is intended to refer to the concentration or dose of a substance (e.g., a compound or a drug) that is required for 50% enhancement or activation of a biological process, or component of a process, including a protein, subunit, organelle, ribonucleoprotein, etc. EC₅₀ also refers to the concentration or dose of a substance that is required for 50% enhancement or activation in vivo, as further defined elsewhere herein. Alternatively, EC₅₀ can refer to the concentration or dose of compound that provokes a response halfway between the baseline and maximum response. The response can be measured in an in vitro or in vivo system as is convenient and appropriate for the biological response of interest. For example, the response can be measured in vitro using cultured muscle cells or in an ex vivo organ culture system with isolated muscle fibers. Alternatively, the response can be measured in vivo using an appropriate research model such as rodent, including mice and rats. The mouse or rat can be an inbred strain with phenotypic characteristics of interest such as obesity or diabetes. As appropriate, the response can be measured in a transgenic or knockout mouse or rat wherein the gene or genes has been introduced or knocked-out, as appropriate, to replicate a disease process.

As used herein, “IC₅₀,” is intended to refer to the concentration or dose of a substance (e.g., a compound or a drug) that is required for 50% inhibition or diminuation of a biological process, or component of a process, including a protein, subunit, organelle, ribonucleoprotein, etc. IC₅₀ also refers to the concentration or dose of a substance that is required for 50% inhibition or diminuation in vivo, as further defined elsewhere herein. Alternatively, IC₅₀ also refers to the half maximal (50%) inhibitory concentration (IC) or inhibitory dose of a substance. The response can be measured in an in vitro or in vivo system as is convenient and appropriate for the biological response of interest. For example, the response can be measured in vitro using cultured muscle cells or in an ex vivo organ culture system with isolated muscle fibers. Alternatively, the response can be measured in vivo using an appropriate research model such as rodent, including mice and rats. The mouse or rat can be an inbred strain with phenotypic characteristics of interest such as obesity or diabetes. As appropriate, the response can be measured in a transgenic or knockout mouse or rat wherein a gene or genes has been introduced or knocked-out, as appropriate, to replicate a disease process.

The term “pharmaceutically acceptable” describes a material that is not biologically or otherwise undesirable, i.e., without causing an unacceptable level of undesirable biological effects or interacting in a deleterious manner.

As used herein, the term “derivative” refers to a compound having a structure derived from the structure of a parent compound (e.g., a compound disclosed herein) and whose structure is sufficiently similar to those disclosed herein and based upon that similarity, would be expected by one skilled in the art to exhibit the same or similar activities and utilities as the claimed compounds, or to induce, as a precursor, the same or similar activities and utilities as the claimed compounds. Exemplary derivatives include salts, esters, amides, salts of esters or amides, and N-oxides of a parent compound.

As used herein, the term “pharmaceutically acceptable carrier” refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions which are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose. Desirably, at least 95% by weight of the particles of the active ingredient have an effective particle size in the range of 0.01 to 10 micrometers.

A residue of a chemical species, as used herein, refers to a derivative of a moiety that is present in a particular product. To form the product, at least one atom of the moiety is replaced by a bond to a second moiety, such that the product contains a derivative of a moiety. For example, in some embodiments, an aromatic residue in a product may refer to one or more —(C₆H₅)ₙ units present in a cyclic peptide described herein. Similarly, an amino acid residue in a product may refer to cyclic peptide described herein having an amino acid incorporated therein through formation of one or more peptide bonds, and such residues may be referred to interchangeably herein as an amino acid or an amino acid residue.

As used herein, the term “chirality” refers to the “D” and “L” isomers of amino acids or amino acid residues.

As used herein, the term “non-aromatic hydrophobic” refers to a moiety that is not soluble in water and which does not comprise an aromatic ring. Generally, neutral moieties and/or non-polar moieties, or moieties that are predominately neutral and/or non-polar are hydrophobic. Hydrophobic can be measured by one of the methods disclosed herein below. Non-aromatic hydrophobic residues include saturated and unsaturated carbocyclyl and heterocyclyl groups which are not aromatic, as well as alkyl, alkenyl, and alkynyl. In some embodiments, the term “non-aromatic hydrophobic” can include groups in which a hydrophobic residue to attached to rest of the molecule through a bonding group which otherwise could be considered to be polar, such as acyl and alkylcarboxamidyl groups as defined below.

As used herein, the term “adjacent” refers to two contiguous amino acids, which are connected by a covalent bond. “Adjacent” is also used interchangeably with “consecutive.”

As used herein “aromatic” refers to an unsaturated cyclic molecule having 4n+2π electrons, wherein n is any integer. The term “non-aromatic” refers to any unsaturated cyclic molecule which does not fall within the definition of aromatic.

The term “acyl” refers to groups —C(O)R, where R is hydrogen, alkyl, alkenyl, alkynyl, carbocyclyl, or heterocyclyl, as defined herein. Unless stated otherwise specifically in the specification, acyl can be optionally substituted.

“Alkyl” or “alkyl group” refers to a fully saturated, straight or branched hydrocarbon chain radical having from one to forty carbon atoms, and which is attached to the rest of the molecule by a single bond. Alkyls comprising any number of carbon atoms from 1 to 20 are included. An alkyl comprising up to 40 carbon atoms is a C₁-C₄₀ alkyl, an alkyl comprising up to 10 carbon atoms is a C₁-C₁₀ alkyl, an alkyl comprising up to 6 carbon atoms is a C₁-C₆ alkyl and an alkyl comprising up to 5 carbon atoms is a C₁-C₅ alkyl. A C₁-C₅alkyl includes C₅ alkyls, C₄ alkyls, C₃ alkyls, C₂ alkyls and C₁ alkyl (i.e., methyl). A C₁-C₆ alkyl includes all moieties described above for C₁-C₅ alkyls but also includes C₆ alkyls. A C₁-C₁₀ alkyl includes all moieties described above for C₁-C₅alkyls and C₁-C₆ alkyls, but also includes C₇, C₈, C₉ and C₁₀ alkyls. Similarly, a C₁-C₁₂ alkyl includes all the foregoing moieties, but also includes C₁₁ and C₁₂ alkyls. Non-limiting examples of C₁-C₁₂ alkyl include methyl. ethyl, n-propyl, i-propyl, sec-propyl, n-butyl, i-butyl, sec-butyl, t-butyl, n-pentyl, t-amyl, n-hexyl, n-heptyl, n-octyl, n-nonyl, n-decyl, n-undecyl, and n-dodecyl. Unless stated otherwise specifically in the specification, an alkyl group can be optionally substituted.

“Alkylene” or “alkylene chain” refers to a fully saturated, straight or branched divalent hydrocarbon chain radical, having from one to forty carbon atoms. Non-limiting examples of C₂-C₄₀ alkylene include ethylene, propylene, n-butylene, ethenylene, propenylene, n-butenylene, propynylene, n-butynylene, and the like. Unless stated otherwise specifically in the specification, an alkylene chain can be optionally substituted. “Alkenyl” or “alkenyl group” refers to a straight or branched hydrocarbon chain radical having from two to forty carbon atoms, and having one or more carbon-carbon double bonds. Each alkenyl group is attached to the rest of the molecule by a single bond. Alkenyl group comprising any number of carbon atoms from 2 to 40 are included. An alkenyl group comprising up to 40 carbon atoms is a C₂-C₄₀ alkenyl, an alkenyl comprising up to 10 carbon atoms is a C₂-C₁₀ alkenyl, an alkenyl group comprising up to 6 carbon atoms is a C₂-C₆ alkenyl and an alkenyl comprising up to 5 carbon atoms is a C₂-C₅ alkenyl. A C₂-C₅ alkenyl includes C₅ alkenyls, C₄ alkenyls, C₃ alkenyls, and C₂ alkenyls. A C₂-C₆ alkenyl includes all moieties described above for C₂-C₅ alkenyls but also includes C₆ alkenyls. A C₂-C₁₀ alkenyl includes all moieties described above for C₂-C₅ alkenyls and C₂-C₆ alkenyls, but also includes C₇, C₈, C₉ and C₁₀ alkenyls. Similarly, a C₂-C₁₂ alkenyl includes all the foregoing moieties, but also includes C₁₁ and C₁₂ alkenyls. Non-limiting examples of C₂-C₁₂ alkenyl include ethenyl (vinyl), 1-propenyl, 2-propenyl (allyl), iso-propenyl, 2-methyl-1-propenyl. 1-butenyl, 2-butenyl, 3-butenyl, 1-pentenyl, 2-pentenyl. 3-pentenyl, 4-pentenyl, 1-hexenyl, 2-hexenyl, 3-hexenyl, 4-hexenyl, 5-hexenyl, 1-heptenyl, 2-heptenyl, 3-heptenyl, 4-heptenyl, 5-heptenyl, 6-heptenyl. 1-octenyl, 2-octenyl, 3-octenyl, 4-octenyl, 5-octenyl, 6-octenyl, 7-octenyl, 1-nonenyl, 2-nonenyl, 3-nonenyl, 4-nonenyl, 5-nonenyl, 6-nonenyl. 7-nonenyl, 8-nonenyl, 1-decenyl, 2-decenyl, 3-decenyl, 4-decenyl, 5-decenyl, 6-decenyl, 7-decenyl, 8-decenyl, 9-decenyl, 1-undecenyl, 2-undecenyl, 3-undecenyl, 4-undecenyl, 5-undecenyl, 6-undecenyl, 7-undecenyl, 8-undecenyl, 9-undecenyl, 10-undecenyl, 1-dodecenyl, 2-dodecenyl, 3-dodecenyl, 4-dodecenyl, 5-dodecenyl, 6-dodecenyl, 7-dodecenyl, 8-dodecenyl, 9-dodecenyl, 10-dodecenyl, and 11-dodecenyl. Unless stated otherwise specifically in the specification, an alkyl group can be optionally substituted.

“Alkenylene” or “alkenylene chain” refers to a straight or branched divalent hydrocarbon chain radical, having from two to forty carbon atoms, and having one or more carbon-carbon double bonds. Non-limiting examples of C₂-C₄₀ alkenylene include ethene, propene, butene, and the like. Unless stated otherwise specifically in the specification, an alkenylene chain can be optionally.

“Alkoxy” refers to the group —OR, where R is alkyl, alkenyl, alkynyl, cycloalkyl, or heterocyclyl as defined herein. Unless stated otherwise specifically in the specification, alkoxy can be optionally substituted.

“Alkylcarbamoyl” refers to the group —O—C(O)—NRₐR_{b}, where Rₐ and R_{b} are the same or different and independently an alkyl, alkenyl, alkynyl, aryl, heteroaryl, cycloalkyl, or heterocyclyl group, as defined herein, or RₐR_{b} can be taken together to form a heterocyclyl group, as defined herein. Unless stated otherwise specifically in the specification, alkylcarbamoyl can be optionally substituted.

“Alkylcarboxamidyl” refers to the group —C(O)—NRₐR_{b}, where Rₐ and R^{b} are the same or different and independently an alkyl, alkenyl, alkynyl, aryl, heteroaryl, cycloalkyl, cycloalkenyl, cycloalkynyl, or heterocyclyl group, as defined herein, or RₐR_{b} can be taken together to form a cycloalkyl group, as defined herein. Unless stated otherwise specifically in the specification, alkylcarboxamidyl can be optionally substituted.

“Alkoxycarbonyl” refers to the group —C(O)OR, where R is alkyl, alkenyl, alkynyl, aryl, heteroaryl, cycloalkyl, cycloalkenyl, cycloalkynyl, or heterocyclyl group, as defined herein. Unless stated otherwise specifically in the specification, alkoxycarbonyl can be optionally substituted.

“Alkylthio” refers to the —SR or —S(O)ₙ₌₁₋₂—R, where R is alkyl, alkenyl, alkynyl, cycloalkyl, cycloalkenyl, cycloalkynyl, or hetereocyclyl, as defined herein. Unless stated otherwise specifically in the specification, alkylthio can be optionally substituted.

“Arylthio” refers to the —SR or —S(O)ₙ₌₁₋₂—R, where R is aryl or hetereoaryl, as defined herein. Unless stated otherwise specifically in the specification, arylthio can be optionally substituted.

“Alkynyl” or “alkynyl group” refers to a straight or branched hydrocarbon chain radical having from two to forty carbon atoms, and having one or more carbon-carbon triple bonds. Each alkynyl group is attached to the rest of the molecule by a single bond. Alkynyl group comprising any number of carbon atoms from 2 to 40 are included. An alkynyl group comprising up to 40 carbon atoms is a C₂-C₄₀ alkynyl, an alkynyl comprising up to 10 carbon atoms is a C₂-C₁₀ alkynyl, an alkynyl group comprising up to 6 carbon atoms is a C₂-C₆ alkynyl and an alkynyl comprising up to 5 carbon atoms is a C₂-C₅ alkynyl. A C₂-C₅ alkynyl includes C₅ alkynyls, C₄ alkynyls, C₃ alkynyls, and C₂ alkynyls. A C₂-C₆ alkynyl includes all moieties described above for C₂-C₅ alkynyls but also includes C₆ alkynyls. A C₂-C₁₀ alkynyl includes all moieties described above for C₂-C₅ alkynyls and C₂-C₆ alkynyls, but also includes C₇, C₈, C₉ and C₁₀ alkynyls. Similarly, a C₂-C₁₂ alkynyl includes all the foregoing moieties, but also includes C₁₁ and C₁₂ alkynyls. Non-limiting examples of C₂-C₁₂ alkenyl include ethynyl, propynyl, butynyl, pentynyl and the like. Unless stated otherwise specifically in the specification, an alkyl group can be optionally substituted.

“Alkynylene” or “alkynylene chain” refers to a straight or branched divalent hydrocarbon chain, having from two to forty carbon atoms, and having one or more carbon-carbon triple bonds. Non-limiting examples of C₂-C₄₀ alkynylene include ethynylene, propargylene and the like. Unless stated otherwise specifically in the specification, an alkynylene chain can be optionally substituted.

“Carbocyclyl,” “carbocyclic ring” or “carbocycle” refers to a rings structure, wherein the atoms which form the ring are each carbon. Carbocyclic rings can comprise from 3 to 20 carbon atoms in the ring. Unless stated otherwise specifically in the specification, the carbocyclyl can be a monocyclic, bicyclic, tricyclic or tetracyclic ring system, which can include fused or bridged ring systems Carbocyclic rings include cycloalkyl, cycloalkenyl, and cycloalkynyl as defined herein. In some embodiments, the carbocyclyl is monovalent and is attached to the rest of molecule through a single bond. In some embodiments, the carbocyclyl is divalent and is independently attached to two moieties through single bonds. Unless stated otherwise specifically in the specification, a carbocyclyl group can be optionally substituted.

“Cycloalkyl” refers to a stable non-aromatic monocyclic or polycyclic fully saturated hydrocarbon radical consisting solely of carbon and hydrogen atoms, which can include fused or bridged ring systems, having from three to twenty carbon atoms, preferably having from three to ten carbon atoms, and which is attached to the rest of the molecule by a single bond. Monocyclic cycloalkyl radicals include, for example, cyclopropyl, cyclobutyl, cyclopentyl, cyclohexyl, cycloheptyl, and cyclooctyl. Polycyclic cycloalkyl radicals include, for example, adamantyl, norbornyl, decalinyl, 7,7-dimethyl-bicyclo[2.2.1]heptanyl, and the like. Unless otherwise stated specifically in the specification, a cycloalkyl group can be optionally substituted.

“Cycloalkenyl” refers to a stable non-aromatic monocyclic or polycyclic hydrocarbon radical consisting solely of carbon and hydrogen atoms, having one or more carbon-carbon double bonds, which can include fused or bridged ring systems, having from three to twenty carbon atoms, preferably having from three to ten carbon atoms, and which is attached to the rest of the molecule by a single bond. Monocyclic cycloalkenyl radicals include, for example, cyclopentenyl, cyclohexenyl, cycloheptenyl, cycloctenyl, and the like. Polycyclic cycloalkenyl radicals include, for example, bicyclo[2.2.1]hept-2-enyl and the like. Unless otherwise stated specifically in the specification, a cycloalkenyl group can be optionally substituted.

“Cycloalkynyl” refers to a stable non-aromatic monocyclic or polycyclic hydrocarbon radical consisting solely of carbon and hydrogen atoms, having one or more carbon-carbon triple bonds, which can include fused or bridged ring systems, having from three to twenty carbon atoms, preferably having from three to ten carbon atoms, and which is attached to the rest of the molecule by a single bond. Monocyclic cycloalkynyl radicals include, for example, cycloheptynyl, cyclooctynyl, and the like. Unless otherwise stated specifically in the specification, a cycloalkynyl group can be optionally substituted.

“Heterocyclyl.” “heterocyclic ring” or “heterocycle” refers to a stable 3- to 20-membered non-aromatic ring radical, which consists of two to fourteen carbon atoms and from one to eight heteroatoms selected from the group consisting of nitrogen, oxygen and sulfur. Unless stated otherwise specifically in the specification, the heterocyclyl radical can be a monocyclic, bicyclic, tricyclic or tetracyclic ring system, which can include fused or bridged ring systems; and the nitrogen, carbon or sulfur atoms in the heterocyclyl radical can be optionally oxidized; the nitrogen atom can be optionally quaternized; and the heterocyclyl radical can be partially or fully saturated. Examples of such heterocyclyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. In some embodiments, the heterocyclyl is monovalent and is attached to the rest of molecule through a single bond. In some embodiments, the heterocyclyl is divalent and is independently attached to two moieties through single bonds. Unless stated otherwise specifically in the specification, a heterocyclyl group can be optionally substituted.

“Aryl” refers to a hydrocarbon ring system radical comprising hydrogen, 6 to 18 carbon atoms and at least one aromatic ring. For purposes of this invention, the aryl radical can be a monocyclic, bicyclic, tricyclic or tetracyclic ring system, which can include fused or bridged ring systems. Aryl radicals include, but are not limited to, aryl radicals derived from aceanthrylene, acenaphthylene, acephenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, the term “aryl” is meant to include aryl radicals that are optionally substituted.

“Aryloxy” refers to groups —OAr, where Ar is an aryl or heteroaryl group as defined herein. Unless otherwise stated specifically in the specification, the aryloxy group can be optionally substituted.

“Heteroaryl” refers to a 5- to 20-membered ring system radical comprising hydrogen atoms, one to thirteen carbon atoms, one to six heteroatoms selected from the group consisting of nitrogen, oxygen and sulfur, and at least one aromatic ring. For purposes of this invention, the heteroaryl radical can be a monocyclic, bicyclic, tricyclic or tetracyclic ring system, which can include fused or bridged ring systems; and the nitrogen, carbon or sulfur atoms in the heteroaryl radical can be optionally oxidized: the nitrogen atom can be optionally quaternized. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e. thienyl). Unless stated otherwise specifically in the specification, a heteroaryl group can be optionally substituted.

“Aralkyl” refers to a radical of the formula —R_{b}—R_{c} where R_{b} is an alkylene, alkenylene or alkynylene group as defined above and Re is one or more aryl radicals as defined above, for example, benzyl, diphenylmethyl and the like. Unless stated otherwise specifically in the specification, an aralkyl group can be optionally substituted.

The term “substituted” used herein means any of the above groups (i.e., alkyl, alkenyl, alkynyl, cycloalkyl, cycloalkenyl, cycloalkynyl, heterocyclyl, aryl, heteroaryl, alkoxy, aryloxy, acyl, alkylcarbamoyl, alkylcarboxamidyl, alkoxy carbonyl, alkylthio, or arylthio) wherein at least one atom is replaced by a non-hydrogen atoms such as, but not limited to: a halogen atom such as F, Cl, Br, and I; an oxygen atom in groups such as hydroxyl groups, alkoxy groups, and ester groups: a sulfur atom in groups such as thiol groups, thioalkyl groups, sulfone groups, sulfonyl groups, and sulfoxide groups: a nitrogen atom in groups such as amines, amides, alkylamines, dialkylamines, arylamines, alkylarylamines, diarylamines, N-oxides, imides, and enamines: a silicon atom in groups such as trialkylsilyl groups, dialkylarylsilyl groups, alkyldiarylsilyl groups, and triarylsilyl groups; and other heteroatoms in various other groups. “Substituted” also means any of the above groups in which one or more atoms are replaced by a higher-order bond (e.g., a double- or triple-bond) to a heteroatom such as oxygen in oxo, carbonyl, carboxyl, and ester groups; and nitrogen in groups such as imines, oximes, hydrazones, and nitriles. For example, “substituted” includes any of the above groups in which one or more atoms are replaced with —NR_{g}Rₕ, —NR_{g}C(═O)Rₕ, —NR_{g}C(═O)NR_{g}Rₕ, —NR_{g}C(═O)ORₕ, —NR_{g}SO₂Rₕ, —OC(═O)NR_{g}Rₕ, —OR_{g}, —SR_{g}, —SOR_{g}, —SO₂R_{g}, —OSO₂R_{g}, —SO₂OR_{g}, ═NSO₂R_{g}, and —SO₂NR_{g}Rₕ. “Substituted also means any of the above groups in which one or more hydrogen atoms are replaced with —C(═O)R_{g}, —C(═O)OR_{g}, —C(═O)NR_{g}Rₕ, —CH₂SO₂R_{g}, —CH₂SO₂NR_{g}Rₕ. In the foregoing, R_{g} and Rₕ are the same or different and independently hydrogen, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl. “Substituted” further means any of the above groups in which one or more atoms are replaced by an amino, cyano, hydroxyl, imino, nitro, oxo, thioxo, halo, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl group. “Substituted” can also mean an amino acid in which one or more atoms on the side chain are replaced by alkyl, alkenyl, alkynyl, acyl, alkylcarboxamidyl, alkoxycarbonyl, carbocyclyl, heterocyclyl, aryl, or heteroaryl. In addition, each of the foregoing substituents can also be optionally substituted with one or more of the above substituents.

As used herein, “linker” or “L” refers to a moiety that covalently attaches two or more components of the polypeptide conjugates disclosed herein (e.g., a linker may covalently attach a CPP and a group that binds to a nucleic acid sequence by electrostatic interactions (i.e., P). In some embodiments, the linker can be natural or non-natural amino acid or polypeptide. In other embodiments, the linker is a synthetic compound containing two or more appropriate functional groups suitable to bind, e.g., the CPP and, independently, P. In some embodiments, the linker is about 3 to about 100 (e.g., about 3 to about 20) atoms in linear length (not counting the branched atoms or substituents). In some embodiments, the linker provides about 1 Å to about 400 Å in distance of the two groups to which it connects. In further examples, the linker is a polyalkyloxide (e.g., polyethyloxide or polyproploxide).

As used herein, “polypeptide” refers to a string of at least two amino acids attached to one another by a peptide bond. There is no upper limit to the number of amino acids that can be included in a polypeptide. Further, polypeptides may include non-natural amino acids, amino acid analogs, or other synthetic molecules that are capable of integrating into a polypeptide.

As used herein, a “monomer” refers to an amino acid residue in a polypeptide. In some embodiments, an amino acid monomer is divalent. In other embodiments, an amino acid monomer may be trivalent if the monomer is further substituted. For example, a cysteine monomer can independently form peptide bonds at the N and C termini, and also form a disulfide bond.

As used herein, an “amino acid-analog” or “analog” (e.g., “arginine-analog”, “lysine-analog” or “histidine-analog”) refers to a variant of an amino acid that retains at least one function of the amino acid, such as the ability to bind an oligonucleotide through electrostatic interactions. Such variants may have an elongated or shorter side chain (e.g., by one or more —CH₂— groups that retains the ability to bind an oligonucleotide through electrostatic interactions, or alternatively, the modification can improve the ability to bind an oligonucleotide through electrostatic interactions. For example, an arginine analog may include an additional methylene or ethylene between the backbone and guanidine/guanidinium group. Other examples include amino acids with one or more additional substituents (e.g., Me, Et, halogen, thiol, methoxy, ethoxy, C1-haloalkyl, C2-haloalkyl, amine, guanidine, etc). The amino acid-analog can be monovalent, divalent, or trivalent.

Examples of arginine-analogs include

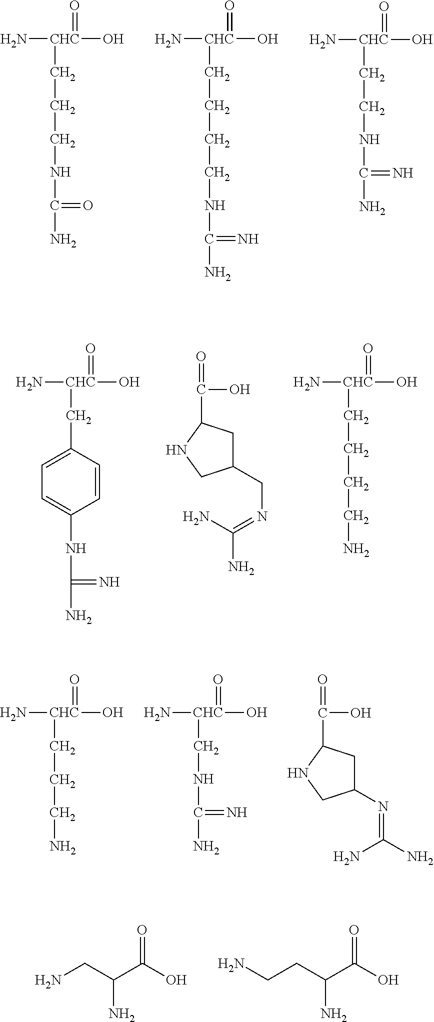

or any of the AAs that contain a quinidine/guanidinium on the side chain group.

Examples of cystine-analogs include homocysteine,

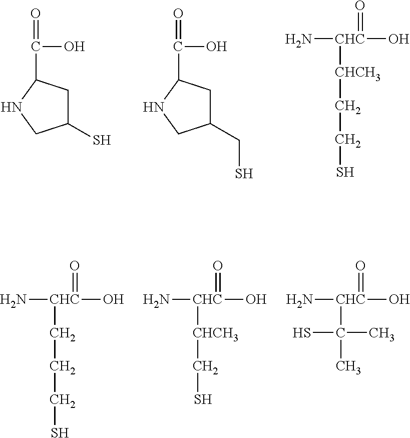

or any of the AAs that contain a thiol group on the side chain.

Throughout the present specification, peptides and amino acid monomers are depicted as charge neutral species. It is to be understood that such species may bear a positive or negative charge depending on the conditions. For example, at pH 7, the N-terminus of an amino acid is protonated and bears a positive charge (—NH₃⁺), and the C-terminus of an amino acid is deprotonated and bears a negative charge (—CO₂⁻). Similarly, the side chains of certain amino acids may bear a positive or negative charge. Cell Penetrating Peptides

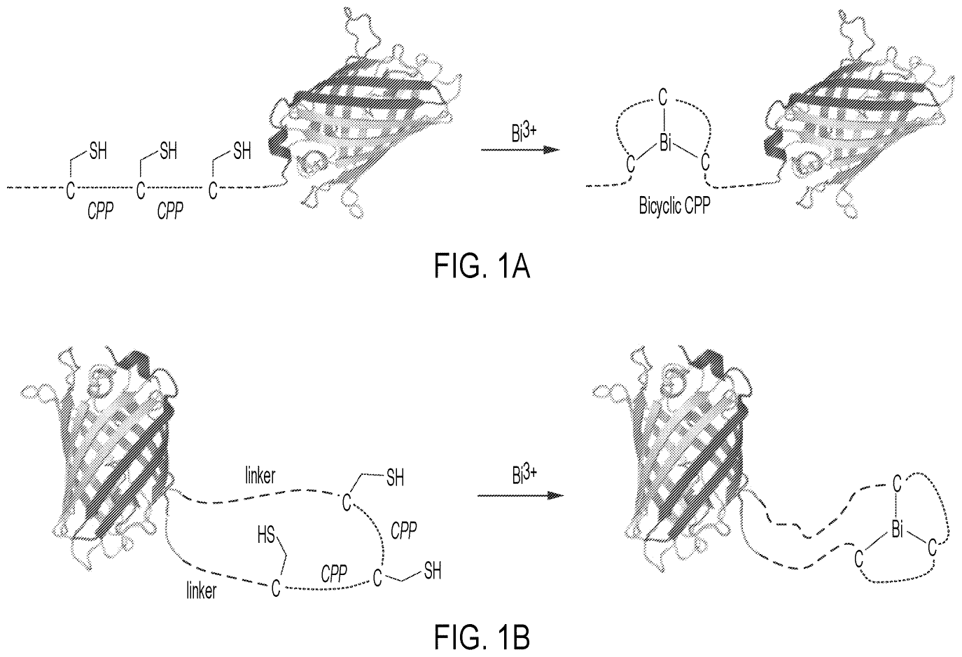

The disclosed peptides take advantage of the unique ability of the bismuth (III) ion to selectively, instantaneously, and quantitatively react with a protein (or a peptide) containing three nearby thiol-containing residues to form a stable metalloprotein (or a metallopeptide) complex (Potocki, S., et al. (2011). Metal binding ability of cysteine-rich peptide domain of ZIP13 Zn²⁺ ions transporter. Inorganic Chemistry, 50 (13), 6135-6145; Voss, S., et al. (2022) Peptide-Bismuth Bicycles: In Situ Access to Stable Constrained Peptides with Superior Bioactivity. Angewandte Chemie (International ed. in English), 61 (4), e202113857). Thus, a protein (or a peptide) is designed to contain a CPP motif with three imbedded thiol-containing residues and produced recombinantly (or chemically) (FIGS. 1A and 1B). After cell lysis, the protein (or peptide), either in a crude cell lysate or after purification, is treated with a BiBr₃ solution to convert the linear CPP sequence into a bicyclic CPP. With properly chosen ring sizes (<10 aa for each ring), the bicyclic CPPs can be highly stable against proteolytic degradation. Cyclization can greatly increase the cell-penetrating activity of the CPP motif. Compared to the previous loop replacement strategy, in which the CPP motif remains “linear” and is restricted to a loop region, the bismuth-based approach disclosed herein generates a bona fide cyclic CPP, which can be fused to the N-terminus, C-terminus, or an internal loop region of a protein. Furthermore, since the bicyclic CPP motif is proteolytically stable, it can be inserted into a surface loop along with a flexible linker sequence on either side (FIG. 1B). The latter can render the approach compatible with a broader range of cargo proteins and loop regions. Additional advantages include the simplicity and efficiency of the cyclization method and the excellent safety of the Bi³⁺ ion, which is already found in FDA-approved drugs (e.g., PEPTO-BISMOL™).

In addition to genetically encoded cyclic CPPs, peptides containing non-proteinogenic amino acids (e.g., Nal and D-amino acids) may also be chemically synthesized and cyclized by the addition of Bi³⁺ ion. The resulting cyclic CPPs can be used to deliver a wide variety of cargoes, as previously demonstrated with cyclic CPPs such as CPP9 and CPP12. Unlike the backbone cyclized CPPs, whose synthesis may be complicated by epimerization of the C-terminal residue during cyclization and the formation of inseparable diastereomers, cyclization by Bi³⁺ is free of epimerization and gives a single product in nearly quantitative yield.

Disclosed herein are peptides having activity as cell penetrating peptides (CPPs). In some embodiments, the peptides comprise a CPP domain of from about 7 to about 25 amino acids in length; wherein the CPP domain comprises any combination of at least two arginines and at least two amino acids having a hydrophobic side chain selected from an aryl or heteroaryl, wherein the aryl and heteroaryl are optionally substituted, and wherein the CPP domain comprises at least three thiol containing residues, e.g., amino acids or related moieties with a thiol containing side chain or a mercaptoacetate, and which are separated from one another by at least one amino acid. In a specific example, one or more (e.g., all) of the thiol-containing residues are cysteine. In other examples one or more (e.g., all) of the thiol-containing residues are homocysteine. In another example, a N-terminal residue can be a 2-mercaptoacetyl residue or higher homologue.

In some embodiments, the CPPs disclosed herein comprise about 8 to about to about 14 amino acids, e.g., about 7, about 8, about 9, about 10, about 11, about 12, about 13, about 14, about 15, about 16, about 17, about 18, about 19, about 20, about 21, about 22, about 22, about 23, about 24, about 25, or about 26 amino acids, inclusive of all ranges and subranges therebetween. In particular embodiments, the CPPs disclosed herein comprise from about 7 to about 12 amino acids, from about 10 to about 20 amino acids, or from about to about 15 amino acids.

Each amino acid can be a natural or non-natural amino acid. The term “non-natural amino acid” refers to an organic compound that is a congener of a natural amino acid in that it has a structure similar to a natural amino acid so that it mimics the structure and reactivity of a natural amino acid. The non-natural amino acid can be a modified amino acid, and/or amino acid analog, that is not one of the 20 common naturally occurring amino acids or the rare natural amino acids selenocysteine or pyrrolysine. Non-natural amino acids can also be the D-isomer of the natural amino acids. Thus, as used herein, the term “amino acid” refers to natural and non-natural amino acids, and analogs and derivatives thereof. Examples of suitable amino acids include, but are not limited to, alanine, allosoleucine, arginine, asparagine, aspartic acid, cysteine, glutamine, glutamic acid, glycine, histidine, isoleucine, leucine, lysine, methionine, napthylalanine, phenylalanine, proline, pyroglutamic acid, serine, threonine, tryptophan, tyrosine, valine, a derivative, or combinations thereof. Analogs of amino acids encompass that have a structural similar but not identical to an amino acid, e.g., due to a modification to the side chain or backbone on said amino acid. Such modifications may increase the hydrophobicity of the side chain, including elongation of the side chain by one or more hydrocarbons, or increasing the solvent accessible surface area (SASA as described herein) of an amino acid having an aromatic ring on its side chain, e.g., by conjugating a second aromatic ring or increasing the size of the aromatic ring. Derivatives of amino acids encompass natural and non-natural amino acids that have been modified (e.g., by substitution) to include a hydrophobic group as described herein. For example, a derivative of lysine includes lysine whose side chain has been substituted with alkylcarboxamidyl. These, and others, are listed in the Table 1 along with their abbreviations used herein.

TABLE 1
Amino Acid Abbreviations

| | Abbreviations* | Abbreviations* |
|---|---|---|
| Amino Acid | L-amino acid | D-amino acid |
| Alanine | Ala (A) | ala (a) |
| Allosoleucine | AIle | Aile |
| Arginine | Arg (R) | arg (r) |
| Asparagine | Asn (N) | asn (n) |
| Aspartic acid | Asp (D) | asp (d) |
| Cysteine | Cys (C) | cys (c) |
| Cyclohexylalanine | Cha | Cha |
| 2,3-diaminopropionic acid | Dap | Dap |
| 4-fluorophenylalanine | Fpa (Σ) | Pfa |
| Glutamic acid | Glu (E) | glu (e) |
| Glutamine | Gln (Q) | gln (q) |
| Glycine | Gly (G) | gly (g) |
| Histidine | His (H) | his (h) |
| Homoproline (aka pipecolic acid) | Pip (Θ) | pip (θ) |
| Isoleucine | Ile (I) | ile (i) |
| Leucine | Leu (L) | leu (l) |
| Lysine | Lys (K) | lys (k) |
| Methionine | Met (M) | met (m) |
| Naphthylalanine | Nal (Φ) | nal (φ) |
| Norleucine | Nle (Ω) | nle |
| Phenylalanine | Phe (F) | phe (f) |
| Phenylglycine | Phg (Ψ) | phg |
| 4-(phosphonodifluoro- | F₂Pmp (Λ) | f₂pmp |
| methyl)phenylalanine | | |
| Proline | Pro (P) | pro (p) |
| Sarcosine | Sar (Ξ) | sar |
| Selenocysteine | Sec (U) | sec (u) |
| Serine | Ser (S) | ser (s) |
| Threonine | Thr (T) | thr (y) |
| Tyrosine | Tyr (Y) | tyr (y) |
| Tryptophan | Trp (W) | trp (w) |
| Valine | Val (V) | val (v) |
| 3-(3-benzothienyl)-alanine | Bta | Bta |
| 4-fluorophenylalanine | Fpa or L-Fpa | fpa or D-Fpa |
| 1-naphthylalanine | L-1-Nal or 1-Nal | D-1-Nal or 1-nal |
| 2-naphthylalanine | L-2-Nal or 2-Nal | D-2-Nal or 2-nal |
| 2-pyridylalanine | L-2-Pya or 2-Pya | D-2-Pya or 2- |
| | | pya |
| 4-pyridylalanine | L-4-Pya or 4-Pya | D-4-Pya or 4- |
| | | pya |

*single letter abbreviations: when shown in capital letters herein it indicates the L-amino acid form, when shown in lower case herein it indicates the D-amino acid form.

When the disclosed peptides are contacted with a Bi(III) ion, the thiol containing residues form a bond with the Bi(III) ion, resulting in a bicyclic peptide. The resulting bismuth-cyclized peptide can be represented by the following general structure when the thiol-containing residues are cysteine and/or cysteine-analogs:

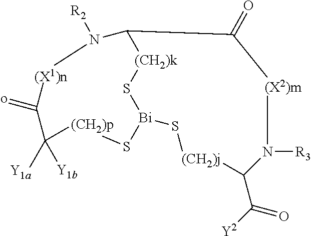

wherein X¹ and X² are amino acids;
n and m are independently selected from 1 to 20;
j and k are independently selected from 1 to 4;
p is selected from 0 to 10;
Y^{1a} and Y^{1b} are independently selected from H, OH, when Y^{1b} is not OH or NR¹Y¹, NR¹Y¹, when Y¹b is not OH or NR¹Y¹, C₁-C₆alkyl, arylC₀-C₆alkyl, heteroarylC₀-C₆alkyl where aryl and heteroaryl are optionally substituted;
Y¹ is H, a protecting group, a counterion, a cargo moiety joined by an optional linker, or a third peptide domain with an optional linker moiety;
R¹, R², and R³ are independently selected from H, C₁-C₆alkyl, arylC₁-C₆alkyl, heteroarylC₁-C₆alkyl where aryl and heteroaryl are optionally substituted;
Y² is OH, NH₂, a protecting group of the carboxylate, a counterion of the carboxylate, a cargo moiety joined by an optional linker, or a third peptide domain with an optional linker moiety;
wherein, the methylene group in (CH₂)p, (CH₂)k or (CH₂)j is optionally substituted with C₁-C₆alkyl, or linked to R¹, R² or R³ to form a ring; and
wherein at least two arginines or arginine-analogs and at least two amino acids having a hydrophobic side chain selected from an aryl or heteroaryl, wherein the aryl and heteroaryl are optionally substituted, are present among (X¹)n and (X²)m.

In the present disclosure, (X¹)n and (X²)m are amino acid sequences of n or m amino acids in length, respectively. As such, (X¹)n and (X²)m are sometimes referred to as peptide domains. That is, each peptide domain is from 2 to 20 amino acids in length in the disclosed CPPs. Further, these two peptide domains are between cysteines or cysteine-analogs such as homocysteines, thus there are at least three cysteines and/or cysteine analogs in the disclosed CPPs.

Together, there are at least two arginines or arginine analogs and at least two amino acids having hydrophobic side chains selected from an aryl or heteroaryl, wherein the aryl and heteroaryl are optionally substituted, in the two peptide domains. In some embodiments, the at least two arginines or arginine analogs are in one of the domains, i.e., (X¹)n or (X²)m. In other embodiments, the at least two arginines or arginine analogs are distributed among the two domains. In some embodiments, the at least two amino acids having hydrophobic side chains selected from an aryl or heteroaryl, wherein the aryl and heteroaryl are optionally substituted, are in one of the domains, i.e., (X¹)n or (X²)m. In other embodiments, the at least two amino acids having hydrophobic side chains selected from an aryl or heteroaryl, wherein the aryl and heteroaryl are optionally substituted are distributed among the two domains. In some embodiments, the peptide domains comprise at least three, at least four, at least five, at least six, or at least seven arginines or arginine analogs. In some embodiments, the peptide domains comprise four arginines or arginine analogs. In some embodiments, the peptide comprises five arginines or arginine analogs. The arginines or arginine analogs can be adjacent to one another or distributed throughout the CPP.

In some embodiments, the peptide domains comprise at least one hydrophobic amino acid having an aryl side chain and at least one hydrophobic amino acid having a heteroaryl side chain. In some embodiments, the cyclic peptide comprises two hydrophobic amino acids having an aryl side chain and one hydrophobic amino acid having a heteroaryl side chain.

In some embodiments, the peptide domains comprise at least two, three, at least four, or at least five hydrophobic amino acids having an aryl or heteroaryl side chain. In some embodiments, the peptide domains comprise at least three, at least four, or at least five consecutive hydrophobic amino acids. In some embodiments, the peptide domains comprise at least two consecutive hydrophobic amino acids. In some embodiments, at least two, at least three, at least four, or at least five consecutive hydrophobic amino acids have alternating chirality. In some embodiments, at least two, at least three, at least four, or at least five consecutive hydrophobic amino acids have the same chirality.

In some embodiments, Y^{1a}, Y^{1b}, Y¹, and/or Y² can be a protecting group such as an acetate or an alkylcarbonate.

In some embodiments, the cell-penetrating peptide domain without cysteine residues, (X¹)n and (X²)m, has at least two block, one block comprising at least two or at least three adjacent amino acids having hydrophobic side chains selected from an aryl or heteroaryl, wherein the aryl and heteroaryl are optionally substituted, another block comprising at least three, at least four, at least five, at least six, or at least seven adjacent arginines or its analogs:

In some embodiments, the first block has two adjacent amino acids having hydrophobic side chains selected from fused aryl, e.g., naphthyl, fused heteroaryl, e.g., benzothienyl, or non-aromatic polycyclic cycloalkyl radicals include, e.g., adamantyl, norbornyl, decalinyl, or 7,7-dimethyl-bicyclo[2.2.1]heptanyl.

In a specific example, R¹, R², and R³ are all H.

In specific examples, j or k are 1, 2, 3, or 4. In other examples, p is 0, 1, 2, 3, 4, 5, 6, 7, 8, 9 or 10.

### Hydrophobic Amino Acids

In some embodiments, the amino acid having a hydrophobic side chain is independently an amino acid having a hydrophobic aromatic side chain. In some embodiments, the aromatic side chain is aryl. In some embodiments, the hydrophobic side chain is heteroaryl. In some embodiments, an amino acid having a hydrophobic aromatic side chain is naphthylalanine, phenylglycine, homophenylalanine, phenylalanine, tryptophan, 3-(3-benzothienyl)-alanine. 3-(2-quinolyl)-alanine, O-benzylserine, 3-(4-(benzyloxy)phenyl)-alanine, S-(4-methylbenzyl) cysteine, N-(naphthalen-2-yl) glutamine, 3-(1,1′-biphenyl-4-yl)-alanine, 3-(3-benzothienyl)-alanine or tyrosine, each of which is optionally substituted with one or more substituents. The structures of a few of these non-natural aromatic hydrophobic amino acids (prior to incorporation into the peptides disclosed herein) are provided below. In particular embodiments, the hydrophobic amino acid is piperidine-2-carboxylate, naphthylalanine, tryptophan, 3-(3-benzothienyl)-alanine, or phenylalanine, each of which is optionally substituted with one or more substituents.

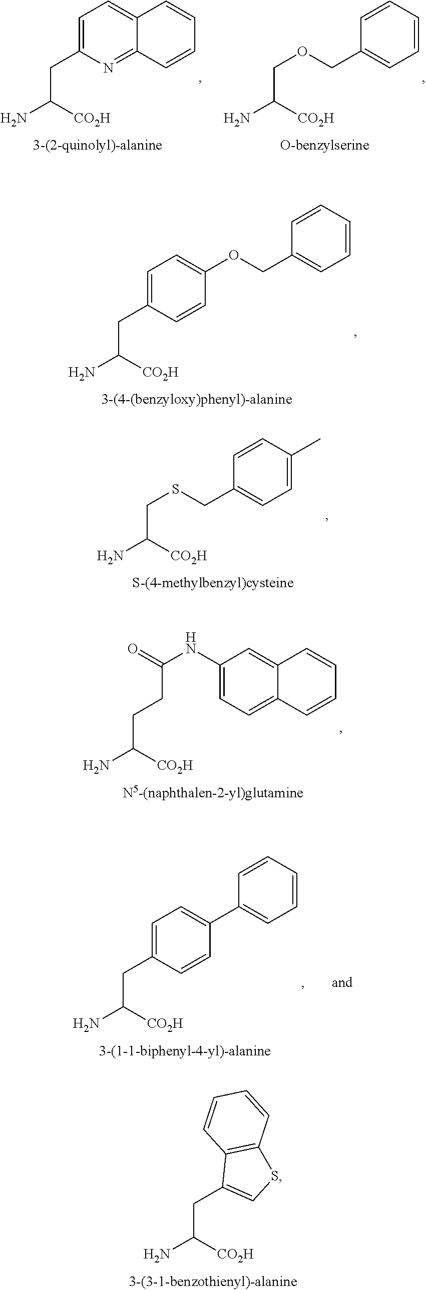

In some embodiments, the cyclic peptide comprises a hydrophobic amino acid selected from the group consisting of L-3-benzothienylalanine, L-4-fluorophenylalanine, D-4-fluorophenylalanine, L-1-naphthylalanine, L-2-naphthylalanine, L-2-pyridylalanine, D-2-pyridylalanine, L-4-pyridylalanine, D-4-pyridylalanine, L-phenylalanine, D-phenylalanine, and combinations thereof. In some embodiments, the cyclic peptide comprises the hydrophobic amino acids L-phenylalanine, D-phenylalanine, and L-2-naphthylalanine. In some embodiments, the cyclic peptide comprises the hydrophobic amino acids L-phenylalanine, D-phenylalanine, and L-3-benzothienylalanine. In some embodiments, the cyclic peptide comprises the hydrophobic amino acids L-phenylalanine, D-4-pyridylalanine, and L-2-napthylalanine.

In some embodiments, each amino acid having a hydrophobic side chain is independently selected from glycine, alanine, valine, leucine, isoleucine, methionine, phenylalanine, tryptophan, proline, naphthylalanine, phenylglycine, homophenylalanine, tyrosine, cyclohexylalanine, piperidine-2-carboxylate, 3-(3-benzothienyl)-alanine, or norleucine, each of which is optionally substituted with one or more substituents.

In some embodiments, the amino acid having a hydrophobic side chain is independently an amino acid having a hydrophobic non-aromatic side chain. In some embodiments, an amino acid having a hydrophobic non-aromatic side chain is alanine, valine, leucine, isoleucine, methionine, or proline. In other embodiments, the amino acid having a hydrophobic non-aromatic side chain has a side chain comprising a C₅-C₄₀ alkyl, alkenyl, alkynyl, acyl, alkylcarboxamidyl, alkoxycarbonyl, carbocyclyl, or heterocyclyl.

Those skilled in the art will appreciate that the N- and/or C-termini of the above non-natural aromatic hydrophobic amino acids, upon incorporation into the peptides disclosed herein, form amide bonds.

The optional substituent can be any atom or group which does not significantly reduce the cytosolic delivery efficiency of the CPP, e.g., a substituent that does not reduce the relative cytosolic delivery efficiency to less than that of c(FΦRRRRQ). In some embodiments, the optional substituent can be a hydrophobic substituent or a hydrophilic substituent. In certain embodiments, the optional substituent is a hydrophobic substituent. In some embodiments, the substituent increases the solvent-accessible surface area (as defined herein below) of the hydrophobic amino acid. In some embodiments, the substituent can be a halogen, alkyl, alkenyl, alkynyl, cycloalkyl, cycloalkenyl, cycloalkynyl, heterocyclyl, aryl, heteroaryl, alkoxy, aryloxy, acyl, alkylcarbamoyl, alkylcarboxamidyl, alkoxycarbonyl, alkylthio, or arylthio. In some embodiments, the substituent is a halogen.

Amino acids having higher hydrophobicity values can be selected to improve cytosolic delivery efficiency of a CPP relative to amino acids having a lower hydrophobicity value. In some embodiments, each hydrophobic amino acid independently has a hydrophobicity value which is greater than that of glycine. In other embodiments, each hydrophobic amino acid independently is a hydrophobic amino acid having a hydrophobicity value which is greater than that of alanine. In still other embodiments, each hydrophobic amino acid independently has a hydrophobicity value which is greater or equal to that of phenylalanine. Hydrophobicity may be measured using hydrophobicity scales known in the art.

In some embodiments, an arginine is adjacent to a hydrophobic amino acid. In some embodiments, the arginine has the same chirality as the hydrophobic amino acid. In some embodiments, at least two arginines are adjacent to each other. In other embodiments, three arginines are adjacent to each other. In some embodiments, at least two hydrophobic amino acids are adjacent to each other. In other embodiments, at least three hydrophobic amino acids are adjacent to each other. In other embodiments, the CPPs described herein comprise at least two consecutive hydrophobic amino acids and at least two adjacent arginines. In further embodiments, one hydrophobic amino acid is adjacent to one of the arginines. In still other embodiments, the CPPs described herein comprise at least three adjacent hydrophobic amino acids and at least three adjacent arginines. In further embodiments, one hydrophobic amino acid is adjacent to one of the arginines. These various combinations of amino acids can have any arrangement of D and L amino acids, e.g., any of the sequences described in the preceding paragraph.

In some embodiments, each hydrophobic amino acid is independently selected from glycine, alanine, valine, leucine, isoleucine, methionine, phenylalanine, tryptophan, proline, naphthylalanine, phenylglycine, homophenylalanine, tyrosine, cyclohexylalanine, piperidine-2-carboxy late, or norleucine, each of which is optionally substituted with one or more substituents.

The chirality of the amino acids (i.e., D or L amino acids) can be selected to improve cytosolic delivery efficiency of the CPP (and the attached cargo as described below). In some embodiments, the hydrophobic amino acid on the N- or C-terminus of an arginine has the same or opposite chirality as the adjacent arginine. In some embodiments, a hydrophobic amino acid has the same chirality as an adjacent arginine. For example, when the arginine is D-arg (i.e. “r”), the hydrophobic amino acid is a D-amino acid, and when the arginine is L-Arg (i.e., “R”), the hydrophobic amino acid is an L-amino acid. Accordingly, in some embodiments, the CPPs disclosed herein may include at least one of the following motifs: D-hydrophobic amino acid-D-arg, D-arg-D-hydrophobic amino acid, L-hydrophobic amino acid-L-Arg, or L-Arg-L-hydrophobic amino acid.

### Cargo

In some embodiments, the CPPs disclosed herein can further include a cargo moiety, which may comprise a peptide. The cargo moiety can comprise one or more detectable moieties, one or more therapeutic moieties, one or more targeting moieties, or any combination thereof. The cargo moiety can also contribute to cell penetration. In some embodiments, the cargo moiety may be a peptide sequence or a non-peptidyl therapeutic agent. In some embodiments, the cargo moiety can be coupled to an amino group (e.g., N-terminus), a carboxy late group (e.g., C-terminus), or a side chain of one or more amino acids in the cCPP. In some embodiments, the CPP and the cargo moiety together are cyclic (referred to herein as “endocyclic”). In the endocyclic system, the cargo moiety can be part of or all of one of the peptide domains (e.g., either (X¹)n or (X²)m). In some embodiments, the cargo moiety can be appended to the CPP (referred to herein as “exocyclic”). In the exocyclic system, the cargo moiety can be Y¹ and/or Y².

In some embodiments, the CPP and cargo sequences can be integrated (inter-mixed) to form “CPP-MP hybrids”, in which the CPP performs dual function of cell entry and target engagement. MP=macrocyclic peptide.

In some embodiments, the cargo moiety can contain a linker (L) to attach to the CPP. In some embodiments, L is 1 to 22 carbon atoms in length, wherein one or more carbon atoms are each optionally and independently replaced by a group selected from C(O), O, N(O), N(alkyl), S, C₂-alkenyl, C₂-alkynyl, cycloalkyl, aryl, heterocyclyl, and heteroaryl. In some embodiments, the linker comprises a polyalkeneoxide. In other embodiments the linker comprises a petide.

The cargo moiety can comprise any cargo of interest, for example a linker moiety, a detectable moiety, a therapeutic moiety, a targeting moiety, and the like, or any combination thereof. In some examples, the cargo moiety can comprise one or more additional amino acids (e.g., K, UK, TRV); a linker (e.g., bifunctional linker LC-SMCC); coenzyme A; phosphocoumaryl amino propionic acid (pCAP); 8-amino-3,6-dioxaoctanoic acid (miniPEG); L-2,3-diaminopropionic acid (Dap or J); L-β-naphthylalanine; L-pipecolic acid (Pip); sarcosine; trimesic acid; 7-amino-4-methylcourmarin (Amc); fluorescein isothiocyanate (FITC); L-2-naphthylalanine; norleucine; 2-aminobutyric acid; Rhodamine B (Rho); Dexamethasone (DEX); or combinations thereof.

In some examples the cargo moiety can comprise any of those listed in Table 2, or derivatives or combinations thereof.

TABLE 2
Example cargo moieties

| SEQ ID NO | Abbreviation | Sequence* |
|---|---|---|
| 2 | R₅ | RRRRR |
| 3 | A₅ | AAAAA |
| 4 | F₄ | FFFF |
| | PCP | DE(pCAP)LI |
| 5 | A₇ | AAAAAAA |
| 6 | | RARAR |
| 7 | | DADAD |
| 8 | | DΩUD |
| 9 | | UTRV |
| | | D-pThr-Pip-Nal |

*pCAP, phosphocoumaryl amino propionic acid; Ω, norleucine; U, 2-aminobutyric acid; D-pThr is D-phosphothreonine, Pip is L-piperidine-2-carboxylate.

### Detectable Moiety

The detectable moiety can comprise any detectable label. Examples of suitable detectable labels include, but are not limited to, a UV-Vis label, a near-infrared label, a luminescent group, a phosphorescent group, a magnetic spin resonance label, a photosensitizer, a photocleavable moiety, a chelating center, a heavy atom, a radioactive isotope, a isotope detectable spin resonance label, a paramagnetic moiety, a chromophore, or any combination thereof. In some embodiments, the label is detectable without the addition of further reagents

In some embodiments, the detectable moiety is a biocompatible detectable moiety, such that the compounds can be suitable for use in a variety of biological applications. “Biocompatible” and “biologically compatible”, as used herein, generally refer to compounds that are, along with any metabolites or degradation products thereof, generally non-toxic to cells and tissues, and which do not cause any significant adverse effects to cells and tissues when cells and tissues are incubated (e.g., cultured) in their presence.

The detectable moiety can contain a luminophore such as a fluorescent label or near-infrared label. Examples of suitable luminophores include, but are not limited to, metal porphyrins: benzoporphyrins: azabenzoporphyrine: napthoporphyrin: phthalocyanine; polycyclic aromatic hydrocarbons such as perylene, perylene diimine, pyrenes: azo dyes; xanthene dyes: boron dipyoromethene, aza-boron dipyoromethene, cyanine dyes, metal-ligand complex such as bipyridine, bipyridyls, phenanthroline, coumarin, and acetylacetonates of ruthenium and iridium: acridine, oxazine derivatives such as benzophenoxazine: aza-annulene, squaraine: 8-hydroxyquinoline, polymethines, luminescent producing nanoparticle, such as quantum dots, nanocrystals: carbostyril; terbium complex; inorganic phosphor; ionophore such as crown ethers affiliated or derivatized dyes: or combinations thereof. Specific examples of suitable luminophores include, but are not limited to, Pd (II) octaethylporphyrin; Pt (II)-octaethylporphyrin; Pd (II) tetraphenylporphyrin; Pt (II) tetraphenylporphyrin; Pd (II) meso-tetraphenylporphyrin tetrabenzoporphine; Pt (II) meso-tetrapheny metrylbenzoporphyrin; Pd (II) octaethylporphyrin ketone; Pt (II) octaethylporphyrin ketone; Pd (II) meso-tetra (pentafluorophenyl) porphyrin; Pt (II) meso-tetra (pentafluorophenyl) porphyrin; Ru (II) tris(4,7-diphenyl-1,10-phenanthroline) (Ru(dpp)₃); Ru (II) tris(1,10-phenanthroline) (Ru(phen)₃), tris(2,2′-bipyridine)ruthenium (II) chloride hexahydrate (Ru(bpy)₃); erythrosine B; fluorescein; fluorescein isothiocyanate (FITC); eosin; iridium (III) ((N-methyl-benzimidazol-2-yl)-7-(diethylamino)-coumarin)); indium (III) ((benzothiazol-2-yl)-7-(diethylamino)-coumarin))-2-(acetylacetonate); Lumogen dyes; Macroflex fluorescent red; Macrolex fluorescent yellow; Texas Red; rhodamine B; rhodamine 6G; sulfur rhodamine; m-cresol; thymol blue; xylenol blue; cresol red; chlorophenol blue; bromocresol green; bromcresol red; bromothymol blue; Cy2; a Cy3; a Cy5; a Cy5.5; Cy7; 4-nitrophenol; alizarin; phenolphthalein; o-cresolphthalein; chlorophenol red; calmagite; bromo-xylenol; phenol red; neutral red; nitrazine; 3,4,5,6-tetrabromphenolphtalein; congo red; fluorescein; eosin; 2′,7′-dichlorofluorescein; 5 (6)-carboxy-fluorecsein; carboxynaphthofluorescein; 8-hydroxypyrene-1,3,6-trisulfonic acid; semi-naphthorhodafluor; semi-naphthofluorescein; tris(4,7-diphenyl-1,10-phenanthroline) ruthenium (II) dichloride; (4,7-diphenyl-1,10-phenanthroline) ruthenium (II) tetraphenylboron; platinum (II) octaethylporphyin; dialkylcarbocyanine; dioctadecylcycloxacarbocyanine; fluorenylmethyloxy carbonyl chloride; 7-amino-4-methylcourmarin (Amc); green fluorescent protein (GFP); and derivatives or combinations thereof.

In some examples, the detectable moiety can comprise Rhodamine B (Rho), fluorescein isothiocyanate (FITC), 7-amino-4-methylcourmarin (Amc), green fluorescent protein (GFP), naphthofluorescein (NF), or derivatives or combinations thereof.

The detectible moiety can be attached to the cell penetrating peptide moiety at the amino group, the carboxylate group, or the side chain of any of the amino acids of the cell penetrating peptide moiety (e.g., at the amino group, the carboxy late group, or the side chain of any amino acid in the CPP).

### Therapeutic Moiety

The disclosed compounds can also comprise a therapeutic moiety. In some examples, the cargo moiety comprises a therapeutic moiety. The detectable moiety can be linked to a therapeutic moiety or the detectable moiety can also serve as the therapeutic moiety. Therapeutic moiety refers to a group that when administered to a subject will reduce one or more symptoms of a disease or disorder.

The therapeutic moiety can comprise a wide variety of drugs, including antagonists, for example enzyme inhibitors, and agonists, for example a transcription factor which results in an increase in the expression of a desirable gene product (although as will be appreciated by those in the art, antagonistic transcription factors can also be used), are all included. In addition, therapeutic moiety includes those agents capable of direct toxicity and/or capable of inducing toxicity towards healthy and/or unhealthy cells in the body. Also, the therapeutic moiety can be capable of inducing and/or priming the immune system against potential pathogens.

The therapeutic moiety can, for example, comprise an anticancer agent, antiviral agent, antimicrobial agent, anti-inflammatory agent, immunosuppressive agent, anesthetics, or any combination thereof.

The therapeutic moiety can comprise an anticancer agent. Example anticancer agents include 13-cis-Retinoic Acid, 2-Amino-6-Mercaptopurine, 2-CdA, 2-Chlorodeoxyadenosine, 5-fluorouracil, 6-Thioguanine, 6-Mercaptopurine, Accutane, Actinomycin-D. Adriamycin, Adrucil, Agrylin, Ala-Cort, Aldesleukin, Alemtuzumab, Alitretinoin, Alkaban-AQ, Alkeran, All-transretinoic acid, Alpha interferon, Altretamine, Amethopterin, Amifostine, Aminoglutethimide, Anagrelide, Anandron, Anastrozole, Arabinosylcytosine. Aranesp. Aredia. Arimidex. Aromasin. Arsenic trioxide. Asparaginase. ATRA, Avastin, BCG, BCNU, Bevacizumab, Bexarotene, Bicalutamide, BiCNU, Blenoxane, Bleomycin, Bortezomib, Busulfan, Busulfex, C225, Calcium Leucovorin, Campath, Camptosar, Camptothecin-11, Capecitabine, Carac, Carboplatin, Carmustine, Carmustine wafer, Casodex, CCNU, CDDP, CeeNU, Cerubidine, cetuximab, Chlorambucil, Cisplatin, Citrovorum Factor, Cladribine. Cortisone, Cosmegen, CPT-11, Cyclophosphamide, Cytadren, Cytarabine, Cytarabine liposomal, Cytosar-U, Cytoxan, Dacarbazine, Dactinomycin, Darbepoetin alfa, Daunomycin, Daunorubicin, Daunorubicin hydrochloride, Daunorubicin liposomal, DaunoXome, Decadron, Delta-Cortef, Deltasone, Denileukin diftitox, DepoCyt, Dexamethasone. Dexamethasone acetate, Dexamethasone sodium phosphate, Dexasone. Dexrazoxane, DHAD, DIC, Diodex, Docetaxel. Doxil, Doxorubicin, Doxorubicin liposomal, Droxia, DTIC, DTIC-Dome, Duralone, Efudex. Eligard, Ellence, Eloxatin, Elspar, Emcyt, Epirubicin, Epoetin alfa, Erbitux, Erwinia L-asparaginase. Estramustine. Ethyol. Etopophos. Etoposide. Etoposide phosphate. Eulexin. Evista, Exemestane. Fareston, Faslodex, Femara, Filgrastim, Floxuridine, Fludara, Fludarabine, Fluoroplex, Fluorouracil, Fluorouracil (cream), Fluoxymesterone, Flutamide, Folinic Acid, FUDR, Fulvestrant, G-CSF, Gefitinib, Gemcitabine, Gemtuzumab ozogamicin, Gemzar, Gleevec, Lupron, Lupron Depot, Matulane, Maxidex, Mechlorethamine, -Mechlorethamine Hydrochlorine, Medralone, Medrol, Megace, Megestrol, Megestrol Acetate, Melphalan, Mercaptopurine, Mesna, Mesnex, Methotrexate, Methotrexate Sodium, Methylprednisolone, Mylocel, Letrozole, Neosar. Neulasta, Neumega, Neupogen, Nilandron, Nilutamide, Nitrogen Mustard. Novaldex, Novantrone, Octreotide, Octreotide acetate, Oncospar, Oncovin. Ontak, Onxal, Oprevelkin, Orapred, Orasone, Oxaliplatin, Paclitaxel, Pamidronate, Panretin, Paraplatin, Pediapred, PEG Interferon, Pegaspargase, Pegfilgrastim, PEG-INTRON, PEG-L-asparaginase. Phenylalanine Mustard, Platinol, Platinol-AQ, Prednisolone, Prednisone, Prelone, Procarbazine, PROCRIT, Proleukin, Prolifeprospan 20 with Carmustine implant, Purinethol, Raloxifene, Rheumatrex, Rituxan, Rituximab, Roveron-A (interferon alfa-2a). Rubex, Rubidomycin hydrochloride, Sandostatin, Sandostatin LAR, Sargramostim, Solu-Cortef, Solu-Medrol, STI-571, Streptozocin, Tamoxifen, Targretin, Taxol. Taxotere, Temodar, Temozolomide, Teniposide, TESPA, Thalidomide, Thalomid, TheraCys, Thioguanine, Thioguanine Tabloid, Thiophosphoamide, Thioplex, Thiotepa, TICE, Toposar, Topotecan, Toremifene, Trastuzumab, Tretinoin, Trexall, Trisenox, TSPA, VCR, Velban, Velcade, VePesid, Vesanoid, Viadur, Vinblastine, Vinblastine Sulfate, Vincasar Pfs, Vincristine, Vinorelbine, Vinorelbine tartrate, VLB, VP-16, Vumon, Xeloda, Zanosar, Zevalin, Zinecard, Zoladex, Zoledronic acid, Zometa, Gliadel wafer, Glivec, GM-CSF, Goserelin, granulocyte colony stimulating factor, Halotestin, Herceptin, Hexadrol, Hexalen, Hexamethylmelamine, HMM, Hycamtin, Hydrea, Hydrocort Acetate, Hydrocortisone, Hydrocortisone sodium phosphate, Hydrocortisone sodium succinate, Hydrocortone phosphate, Hydroxyurea, Ibritumomab, Ibritumomab Tiuxetan, Idamycin, Idarubicin, Ifex, IFN-alpha, Ifosfamide, IL 2, IL-11, Imatinib mesylate, Imidazole Carboxamide, Interferon alfa, Interferon Alfa-2b (PEG conjugate). Interleukin 2, Interleukin-11, Intron A (interferon alfa-2b). Leucovorin, Leukeran, Leukine. Leuprolide, Leurocristine, Leustatin, Liposomal Ara-C, Liquid Pred, Lomustine, L-PAM, L-Sarcolysin, Meticorten, Mitomycin, Mitomycin-C, Mitoxantrone, M-Prednisol, MTC, MTX, Mustargen, Mustine, Mutamycin, Myleran, Iressa, Irinotecan, Isotretinoin, Kidrolase, Lanacort, L-asparaginase, and LCR. The therapeutic moiety can also comprise a biopharmaceutical such as, for example, an antibody.

In some examples, the therapeutic moiety can comprise an antiviral agent, such as ganciclovir, azidothymidine (AZT), lamivudine (3TC), etc.

In some examples, the therapeutic moiety can comprise an antibacterial agent, such as acedapsone; acetosulfone sodium; alamecin; alexidine; amdinocillin; amdinocillin pivoxil; amicycline; amifloxacin; amifloxacin mesylate; amikacin; amikacin sulfate; aminosalicylic acid; aminosalicylate sodium; amoxicillin; amphomycin; ampicillin; ampicillin sodium; apalcillin sodium; apramycin; aspartocin; astromicin sulfate; avilamycin; avoparcin; azithromycin; azlocillin; azlocillin sodium; bacampicillin hydrochloride; bacitracin; bacitracin methylene disalicylate; bacitracin zinc; bambermycins; benzoylpas calcium; berythromycin; betamicin sulfate; biapenem; biniramycin; biphenamine hydrochloride; bispyrithione magsulfex; butikacin; butirosin sulfate; capreomycin sulfate; carbadox; carbenicillin disodium; carbenicillin indanyl sodium; carbenicillin phenyl sodium; carbenicillin potassium; carumonam sodium; cefaclor; cefadroxil; cefamandole; cefamandole nafate; cefamandole sodium; cefaparole; cefatrizine; cefazaflur sodium; cefazolin; cefazolin sodium; cefbuperazone; cefdinir; cefepime; cefepime hydrochloride; cefetecol; cefixime; cefmenoxime hydrochloride; cefmetazole; cefmetazole sodium; cefonicid monosodium; cefonicid sodium; cefoperazone sodium; ceforanide; cefotaxime sodium; cefotetan; cefotetan disodium; cefotiam hydrochloride; cefoxitin; cefoxitin sodium; cefpimizole; cefpimizole sodium; cefpiramide; cefpiramide sodium; cefpirome sulfate; cefpodoxime proxetil; cefprozil; cefroxadine; cefsulodin sodium; ceftazidime; ceftibuten; ceftizoxime sodium; ceftriaxone sodium; cefuroxime; cefuroxime axetil; cefuroxime pivoxetil; cefuroxime sodium; cephacetrile sodium; cephalexin; cephalexin hydrochloride; cephaloglycin; cephaloridine; cephalothin sodium; cephapirin sodium; cephradine; cetocy cline hydrochloride; cetophenicol; chloramphenicol; chloramphenicol palmitate; chloramphenicol pantothenate complex; chloramphenicol sodium succinate; chlorhexidine phosphanilate; chlorovylenol; chlortetracycline bisulfate; chlortetracycline hydrochloride; cinoxacin; ciprofloxacin; ciprofloxacin hydrochloride; cirolemycin; clarithromycin; clinafloxacin hydrochloride; clindamycin; clindamycin hydrochloride; clindamycin palmitate hydrochloride; clindamycin phosphate; clofazimine; cloxacillin benzathine; cloxacillin sodium; cloxyquin; colistimethate sodium; colistin sulfate; coumermycin; coumermycin sodium; cyclacillin; cycloserine; dalfopristin; dapsone; daptomycin; demeclocycline; demeclocycline hydrochloride; demecycline; denofungin; diaveridine; dicloxacillin; dicloxacillin sodium; dihydrostreptomycin sulfate; dipyrithione; dirithromycin; doxycycline; doxycycline calcium; doxycycline fosfatex; doxycycline hyclate; droxacin sodium; enoxacin; epicillin; epitetracycline hydrochloride; erythromycin; erythromycin acistrate; erythromycin estolate; erythromycin ethylsuccinate; erythromycin gluceptate; erythromycin lactobionate; erythromycin propionate; erythromycin stearate; ethambutol hydrochloride; ethionamide; fleroxacin; floxacillin; fludalanine; flumequine; fosfomycin; fosfomycin tromethamine; fumoxicillin; furazolium chloride; furazolium tartrate; fusidate sodium; fusidic acid; gentamicin sulfate; gloximonam; gramicidin; haloprogin; hetacillin; hetacillin potassium; hexedine; ibafloxacin; imipenem; isoconazole; isepamicin; isoniazid; josamycin; kanamycin sulfate; kitasamycin; levofuraltadone; levopropylcillin potassium; lexithromycin; lincomycin; lincomycin hydrochloride; lomefloxacin; Lomefloxacin hydrochloride; lomefloxacin mesylate; loracarbef; mafenide; meclocycline; meclocycline sulfosalicylate; megalomicin potassium phosphate; mequidox; meropenem; methacycline; methacycline hydrochloride; methenamine; methenamine hippurate; methenamine mandelate; methicillin sodium; metioprim; metronidazole hydrochloride; metronidazole phosphate; mezlocillin; mezlocillin sodium; minocycline; minocycline hydrochloride; mirincamycin hydrochloride; monensin; monensin sodiumr; nafcillin sodium; nalidixate sodium; nalidixic acid; natainycin; nebramycin; neomycin palmitate; neomycin sulfate; neomycin undecylenate; netilmicin sulfate; neutramycin; nifuiradene; nifuraldezone; nifuratel; nifuratrone; nifurdazil; nifurimide; nifiupirinol; nifurquinazol; nifurthiazole; nitrocycline; nitrofurantoin; nitromide; norfloxacin; novobiocin sodium; ofloxacin; onnetoprim; oxacillin; oxacillin sodium; oximonam; oximonam sodium; oxolinic acid; oxytetracycline; oxytetracycline calcium; oxytetracycline hydrochloride; paldimycin; parachlorophenol; paulomycin; pefloxacin; pefloxacin mesylate; penamecillin; penicillin G benzathine; penicillin G potassium; penicillin G procaine; penicillin G sodium; penicillin V; penicillin V benzathine; penicillin V hydrabamine; penicillin V potassium, pentizidone sodium; phenyl aminosalicylate; piperacillin sodium; pirbenicillin sodium; piridicillin sodium; pirlimycin hydrochloride; pivampicillin hydrochloride; pivampicillin pamoate; pivampicillin probenate; polymyxin B sulfate; porfiromycin; propikacin; pyrazinamide; pyrithione zinc; quindecamine acetate; quinupristin; racephenicol; ramoplanin; ranimycin; relomycin; repromicin; rifabutin; rifametane; rifamexil; rifamide; rifampin; rifapentine; rifaximin; rolitetracycline; rolitetracycline nitrate; rosaramicin; rosaramicin butyrate; rosaramicin propionate; rosaramicin sodium phosphate; rosaramicin stearate; rosoxacin; roxarsone; roxithromycin; sancycline; sanfetrinem sodium; sarmoxicillin; sarpicillin; scopafungin; sisomicin; sisomicin sulfate; sparfloxacin; spectinomycin hydrochloride; spiramycin; stallimycin hydrochloride; steffimycin; streptomycin sulfate; streptonicozid; sulfabenz; sulfabenzamide; sulfacetamide; sulfacetamide sodium; sulfacytine; sulfadiazine; sulfadiazine sodium; sulfadoxine; sulfalene; sulfamerazine; sulfameter; sulfamethazine; sulfamethizole; sulfamethoxazole; sulfamonomethoxine; sulfamoxole; sulfanilate zinc; sulfanitran; sulfasalazine; sulfasomizole; sulfathiazole; sulfazamet; sulfisoxazole; sulfisoxazole acetyl; sulfisboxazole diolamine; sulfomyxin; sulopenem; sultamricillin; suncillin sodium; talampicillin hydrochloride; teicoplanin; temafloxacin hydrochloride; temocillin; tetracycline; tetracycline hydrochloride; tetracycline phosphate complex; tetroxoprim; thiamphenicol; thiphencillin potassium; ticarcillin cresyl sodium; ticarcillin disodium; ticarcillin monosodium; ticlatone; tiodonium chloride; tobramycin; tobramycin sulfate; tosufloxacin; trimethoprim; trimethoprim sulfate; trisulfapyrimidines; troleandomycin; trospectomycin sulfate; tyrothricin; vancomycin; vancomycin hydrochloride; virginiamycin; or zorbamycin.

In other examples, the therapeutic moiety comprises a therapeutic protein. For example, some people have defects in certain enzymes (e.g., lysosomal storage disease). It is disclosed herein to deliver such enzymes/proteins to human cells by linking to the enzyme/protein to one of the disclosed cell penetrating peptides. In a specific example, the therapeutic moiety is a Keap1 inhibitor, K1.

In some examples, the therapeutic moiety comprises a targeting moiety. The targeting moiety can comprise, for example, a sequence of amino acids that can target one or more enzyme domains. In some examples, the targeting moiety can comprise an inhibitor against an enzyme that can play a role in a disease, such as cancer, cystic fibrosis, diabetes, obesity, or combinations thereof. For example, the targeting moiety can comprise any of the sequences listed in Table 3.

TABLE 3
Example targeting moieties

| Abbreviation* | Sequence |
|---|---|
| PΘGΛYR | Pro-Pip-Gly-F₂Pmp-Tyr-Arg |
| SΘIΛΛR | Ser-Pip-Ile-F₂Pmp-F₂Pmp-Arg |
| IHIΛIR | Ile-His-Ile-F₂Pmp-Ile-Arg |
| AaIΛΘR | Ala-(D-Ala)-Ile-F₂Pmp-Pip-Arg |
| ΣSΘΛvR | Fpa-Ser-Pip-F₂Pmp-(D-Val)-Arg |
| ΘnPΛAR | Pip-(D-Asn)-Pro-F₂Pmp-Ala-Arg |
| TΨAΛGR | Tyr-Phg-Ala-F₂Pmp-Gly-Arg |
| AHIΛaR | Ala-His-Ile-F₂Pmp-(D-Ala)-Arg |
| GnGΛpR | Gly-(D-Asn)-Gly-F₂Pmp-(D-Pro)-Arg |
| fQΘΛIR | (D-Phe)-Gln-Pip-F₂Pmp-Ile-Arg |
| SPGΛHR | Ser-Pro-Gly-F₂Pmp-His-Arg |
| ΘYIΛHR | Pip-Tyr-Ile-F₂Pmp-His-Arg |
| SvPΛHR | Ser-(D-Val)-Pro-F₂Pmp-His-Arg |
| AIPΛnR | Ala-Ile-Pro-F₂Pmp-(D-Asn)-Arg |
| ΣSIΛQF | Fpa-Ser-Ile-F₂Pmp-Gln-Arg |
| AaΨΛfR | Ala-(D-Ala)-Phg-F₂Pmp-(D-Phe)-Arg |
| ntΨΛΨR | (D-Asn)-(D-Thr)-Phg-F₂Pmp-Phg-Arg |
| IPΨΛΩR | Ile-Pro-Phg-F₂Pmp-Nle-Arg |
| QΘΣΛΘR | Gln-Pip-Fpa-F₂Pmp-Pip-Arg |
| nAΣΛGR | (D-Asn)-Ala-Fpa-F₂Pmp-Gly-Arg |
| ntYΛAR | (D-Asn)-(D-Thr)-Tyr-F₂Pmp-Ala-Arg |
| eAΨΛvR | (D-Glu)-Ala-Phg-F₂Pmp-(D-Val)-Arg |
| IvΨΛAR | Ile-(D-Val)-Phg-F₂Pmp-Ala-Arg |
| YtΨΛAR | Tyr-(D-Thr)-Phg-F₂Pmp-Ala-Arg |
| nΘΨΛIR | (D-Asn)-Pip-Phg-F₂Pmp-Ile-Arg |
| ΘnWΛHR | Pip-(D-Asn)-Trp-F₂Pmp-His-Arg |
| YΘvΛIR | Tyr-Pip-(D-Val)-F₂Pmp-Ile-Arg |
| nSAΛGR | (D-Asn)-Ser-(D-Ala)-F₂Pmp-Gly-Arg |
| tnvΛaR | (D-Thr)-(D-Asn)-(D-Val)-F₂Pmp-(D-Ala)-Arg |
| ntvΛtR | (D-Asn)-(D-Thr)-(D-Val)-F₂Pmp-(D-Thr)-Arg |
| SItΛYR | Ser-Ile-(D-Thr)-F₂Pmp-Tyr-Arg |
| nΣnΛlR | (D-Asn)-Fpa-(D-Asn)-F₂Pmp-(D-Leu)-Arg |
| ΥnnΛΩR | Tyr-(D-Asn)-(D-Asn)-F₂Pmp-Nle-Arg |
| nYnΛGR | (D-Asn)-Tyr-(D-Asn)-F₂Pmp-Gly-Arg |
| AWnΛAR | Ala-Trp-(D-Asn)-F₂Pmp-Ala-Arg |
| vtHΛYR | (D-Val)-(D-Thr)-His-F₂Pmp-Tyr-Arg |
| PΨHΛΘR | Pro-Phg-His-F₂Pmp-Pip-Arg |
| nΨHΛGR | (D-Asn)-Phg-His-F₂Pmp-Gly-Arg |
| PAHΛGR | Pro-Ala-His-F₂Pmp-Gly-Arg |
| AYHΛIR | Ala-Tyr-His-F₂Pmp-Ile-Arg |
| nΘeΛYR | (D-Asn)-Pip-(D-Glu)-F₂Pmp-Tyr-Arg |
| vSSΛtR | (D-Val)-Ser-Ser-F₂Pmp-(D-Thr)-Arg |
| aΞt′ϑΦ′YNK | ((D-Ala)-Sar-(D-pThr)-Pp-Nal-Tyr-Gln)-Lys |
| Tm(aΞt′ϑΦ′RA)Dap | Tm((D-Ala)-Sar-(D-pThr)-Pp-Nal-Arg-Ala)-Dap |
| Tm(aΞt′ϑΦ′RAa)Dap | Tm((D-Ala)-Sar-(D-pThr)-Pp-Nal-Arg-Ala-(D- |
| | Ala))-Dap |
| Tm(aΞtϑΦ′RAa)Dap | Tm((D-Ala)-Sar-(D-Thr)-Pp-Nal-Arg-Ala-(D- |
| | Ala))-Dap |
| Tm(aΞtaΦ′RAa)Dap | Tm((D-Ala)-Sar-(D-Thr)-(D-Ala)-Nal-Arg-Ala-(D- |
| | Ala))-Dap |

*Fpa, Σ: L-4-fluorophenylalanine; Pip, Θ: L-homoproline; Nle, Ω: L-norleucine; Phg, Ψ L-phenylglycine; F₂Pmp, Λ: L-4-(phosphonodifluoromethyl)phenylalanine; Dap, L-2,3-diaminopropionic acid; Nal, Φ: L-β-naphthylalanine; Pp, ϑ: L-pipecolic acid; Sar, Ξ: sarcosine; Tm, trimesic acid.

The targeting moiety and cell penetrating peptide moiety can overlap. That is, the residues that form the cell penetrating peptide moiety can also be part of the sequence that forms the targeting moiety, and vice a versa.

The therapeutic moiety can be attached to the cell penetrating peptide moiety at the amino group, the carboxylate group, or the side chain of any of the amino acids of the cell penetrating peptide moiety (e.g., at the amino group, the carboxylate group, or the side chain or any of amino acid of the CPP). In some examples, the therapeutic moiety can be attached to the detectable moiety.

In some examples, the therapeutic moiety can comprise a targeting moiety that can act as an inhibitor against Ras (e.g., K-Ras), PTP1B, Pin1, Grb2 SH2, CAL PDZ, and the like, or combinations thereof.

In some embodiments, the therapeutic moiety is a nucleic acid. In some embodiments, the nucleic acid is an antisense compound. In some embodiments, the antisense compound is selected from the group consisting of an antisense oligonucleotide, a small interfering RNA (siRNA), microRNA (miRNA), a ribozyme, an immune stimulating nucleic acid, an antagomir, an antimir, a microRNA mimic, a supermir, a U1 adaptor, and an aptamer. In some embodiments, the nucleic acid is a peptide nucleic acid (PNA) and a phosphorodiamidate morpholino oligomer (PMO).

Also disclosed herein are pharmaceutically-acceptable salts and prodrugs of the disclosed compounds. Pharmaceutically-acceptable salts include salts of the disclosed compounds that are prepared with acids or bases, depending on the particular substituents found on the compounds. Under conditions where the compounds disclosed herein are sufficiently basic or acidic to form stable nontoxic acid or base salts, administration of the compounds as salts can be appropriate. Examples of pharmaceutically-acceptable base addition salts include sodium, potassium, calcium, ammonium, or magnesium salt. Examples of physiologically-acceptable acid addition salts include hydrochloric, hydrobromic, nitric, phosphoric, carbonic, sulfuric, and organic acids like acetic, propionic, benzoic, succinic, fumaric, mandelic, oxalic, citric, tartaric, malonic, ascorbic, alpha-ketoglutaric, alpha-glycophosphoric, maleic, tosyl acid, methanesulfonic, and the like. Thus, disclosed herein are the hydrochloride, nitrate, phosphate, carbonate, bicarbonate, sulfate, acetate, propionate, benzoate, succinate, fumarate, mandelate, oxalate, citrate, tartarate, malonate, ascorbate, alpha-ketoglutarate, alpha-glycophosphate, maleate, tosylate, and mesylate salts. Pharmaceutically acceptable salts of a compound can be obtained using standard procedures well known in the art, for example, by reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made.

### Methods of Making

The compounds described herein can be prepared in a variety of ways known to one skilled in the art of organic synthesis or variations thereon as appreciated by those skilled in the art. The compounds described herein can be prepared from readily available starting materials. Optimum reaction conditions can vary with the particular reactants or solvents used, but such conditions can be determined by one skilled in the art.

Variations on the compounds described herein include the addition, subtraction, or movement of the various constituents as described for each compound. Similarly, when one or more chiral centers are present in a molecule, the chirality of the molecule can be changed. Additionally, compound synthesis can involve the protection and deprotection of various chemical groups. The use of protection and deprotection, and the selection of appropriate protecting groups can be determined by one skilled in the art. The chemistry of protecting groups can be found, for example, in Wuts and Greene, Protective Groups in Organic Synthesis, 4th Ed., Wiley & Sons, 2006, which is incorporated herein by reference in its entirety.

The starting materials and reagents used in preparing the disclosed compounds and compositions are either available from commercial suppliers such as Aldrich Chemical Co., (Milwaukee, WI), Acros Organics (Morris Plains, NJ), Fisher Scientific (Pittsburgh, PA), Sigma (St. Louis, MO), Pfizer (New York, NY), GlaxoSmithKline (Raleigh, NC). Merck (Whitehouse Station, NJ). Johnson & Johnson (New Brunswick, NJ). Aventis (Bridgewater. NJ), AstraZeneca (Wilmington, DE), Novartis (Basel, Switzerland), Wyeth (Madison. NJ), Bristol-Myers-Squibb (New York, NY), Roche (Basel, Switzerland), Lilly (Indianapolis, IN), Abbott (Abbott Park. Ill.), Schering Plough (Kenilworth, NJ), or Boehringer Ingelheim (Ingelheim, Germany), or are prepared by methods known to those skilled in the art following procedures set forth in references such as Fieser and Fieser's Reagents for Organic Synthesis, Volumes 1-17 (John Wiley and Sons. 1991); Rodd's Chemistry of Carbon Compounds, Volumes 1-5 and Supplementals (Elsevier Science Publishers, 1989); Organic Reactions, Volumes 1-40 (John Wiley and Sons, 1991); March's Advanced Organic Chemistry, (John Wiley and Sons, 4th Edition); and Larock's Comprehensive Organic Transformations (VCH Publishers Inc., 1989). Other materials, such as the pharmaceutical carriers disclosed herein can be obtained from commercial sources.

Reactions to produce the compounds described herein can be carried out in solvents, which can be selected by one of skill in the art of organic synthesis. Solvents can be substantially nonreactive with the starting materials (reactants), the intermediates, or products under the conditions at which the reactions are carried out, i.e., temperature and pressure. Reactions can be carried out in one solvent or a mixture of more than one solvent. Product or intermediate formation can be monitored according to any suitable method known in the art. For example, product formation can be monitored by spectroscopic means, such as nuclear magnetic resonance spectroscopy (e.g., ¹H or ¹³C) infrared spectroscopy, spectrophotometry (e.g., UV-visible), or mass spectrometry, or by chromatography such as high performance liquid chromatography (HPLC) or thin layer chromatography.

The disclosed compounds can be prepared by solid phase peptide synthesis wherein the amino acid α-N-terminus is protected by an acid or base protecting group. Such protecting groups should have the properties of being stable to the conditions of peptide linkage formation while being readily removable without destruction of the growing peptide chain or racemization of any of the chiral centers contained therein. Suitable protecting groups are 9-fluorenylmethyloxy carbonyl (Fmoc), t-butyloxy carbonyl (Boc), benzyloxy carbonyl (Cbz), biphenylisopropyloxycarbonyl, t-amyloxycarbonyl, isobornyloxy carbonyl, α,α-dimethyl-3,5-dimethoxy benzyloxy carbonyl, o-nitrophenylsulfenyl, 2-cyano-t-butyloxy carbonyl, and the like. The 9-fluorenylmethyloxy carbonyl (Fmoc) protecting group is particularly preferred for the synthesis of the disclosed compounds. Other preferred side chain protecting groups are, for side chain amino groups like lysine and arginine, 2,2,5,7,8-pentamethylchroman-6-sulfonyl (pmc), nitro, p-toluenesulfonyl, 4-methoxy benzene-sulfonyl, Cbz, Boc, and adamantyloxy carbonyl; for tyrosine, benzyl, o-bromobenzyloxy-carbonyl, 2,6-dichlorobenzyl, isopropyl, t-butyl (t-Bu), cyclohexyl, cyclopenyl and acetyl (Ac); for serine, t-butyl, benzyl and tetrahydropyranyl; for histidine, trityl, benzyl, Cbz, p-toluenesulfonyl and 2,4-dinitrophenyl; for tryptophan, formyl; for aspartic acid and glutamic acid, benzyl and t-butyl and for cysteine, triphenylmethyl (trityl).

In the solid phase peptide synthesis method, the α-C-terminal amino acid is attached to a suitable solid support or resin. Suitable solid supports useful for the above synthesis are those materials which are inert to the reagents and reaction conditions of the stepwise condensation-deprotection reactions, as well as being insoluble in the media used. Solid supports for synthesis of α-C-terminal carboxy peptides is 4-hydroxy methylphenoxymethyl-copoly(styrene-1% divinylbenzene) or 4-(2′,4′-dimethoxyphenyl-Fmoc-aminomethyl)phenoxyacetamidoethyl resin available from Applied Biosystems (Foster City, Calif.). The α-C-terminal amino acid is coupled to the resin by means of N,N′-dicyclohexylcarbodiimide (DCC), N,N′-diisopropylcarbodiimide (DIC) or O-benzotriazol-1-yl-N,N,N′,N′-tetramethyluroniumhexafluorophosphate (HBTU), with or without 4-dimethylaminopyridine (DMAP), 1-hydroxy benzotriazole (HOBT), benzotriazol-1-yloxy-tris(dimethylamino)phosphoniumhexafluorophosphate (BOP) or bis(2-oxo-3-oxazolidinyl)phosphine chloride (BOPCl), mediated coupling for from about 1 to about 24 hours at a temperature of between 10° C. and 50° C. in a solvent such as dichloromethane or DMF. When the solid support is 4-(2′,4′-dimethoxyphenyl-Fmoc-aminomethyl) phenoxy-acetamidoethyl resin, the Fmoc group is cleaved with a secondary amine, preferably piperidine, prior to coupling with the α-C-terminal amino acid as described above. One method for coupling to the deprotected 4 (2′,4′-dimethoxyphenyl-Fmoc-aminomethyl) phenoxy-acetamidoethyl resin is O-benzotriazol-1-yl-N,N,N′,N′-tetramethyluroniumhexafluorophosphate (HBTU, 1 equiv.) and 1-hydroxy benzotriazole (HOBT, 1 equiv.) in DMF. The coupling of successive protected amino acids can be carried out in an automatic polypeptide synthesizer. In one example, the α-N-terminus in the amino acids of the growing peptide chain are protected with Fmoc. The removal of the Fmoc protecting group from the α-N-terminal side of the growing peptide is accomplished by treatment with a secondary amine, preferably piperidine. Each protected amino acid is then introduced in about 3-fold molar excess, and the coupling is preferably carried out in DMF. The coupling agent can be O-benzotriazol-1-yl-N,N,N′,N′-tetramethyluroniumhexafluorophosphate (HBTU, 1 equiv.) and 1-hydroxy benzotriazole (HOBT, 1 equiv.). At the end of the solid phase synthesis, the polypeptide is removed from the resin and deprotected, either successively or in a single operation. Removal of the polypeptide and deprotection can be accomplished in a single operation by treating the resin-bound polypeptide with a cleavage reagent comprising thioanisole, water, ethanedithiol and trifluoroacetic acid. In cases wherein the α-C-terminal of the polypeptide is an alkylamide, the resin is cleaved by aminolysis with an alkylamine. Alternatively, the peptide can be removed by transesterification, e.g. with methanol, followed by aminolysis or by direct transamidation. The protected peptide can be purified at this point or taken to the next step directly. The removal of the side chain protecting groups can be accomplished using the cleavage cocktail described above. The fully deprotected peptide can be purified by a sequence of chromatographic steps employing any or all of the following types: ion exchange on a weakly basic resin (acetate form); hydrophobic adsorption chromatography on underivatized polystyrene-divinylbenzene (for example, Amberlite XAD); silica gel adsorption chromatography; ion exchange chromatography on carboxymethylcellulose; partition chromatography, e.g. on Sephadex G-25, LH-20 or countercurrent distribution; high performance liquid chromatography (HPLC), especially reverse-phase HPLC on octyl- or octadecylsilyl-silica bonded phase column packing.

The disclosed peptides can be made to cyclize by contacting the peptide with a bismuth salt, such as BiCl₃, BiBr₃, Bi₂O₃, BiNO₃, Bi(OAc)₃, Bi₂(SO₄)₃, Bi(CO₂)₃, BiPO₄, and the like.

### Methods of Use

Also provided herein are methods of use of the compounds or compositions described herein. Also provided herein are methods for treating a disease or pathology in a subject in need thereof comprising administering to the subject an effective amount of any of the compounds or compositions described herein.

Also provided herein are methods of treating cancer in a subject. The methods include administering to a subject an effective amount of one or more of the compounds or compositions described herein, or a pharmaceutically acceptable salt thereof. The compounds and compositions described herein or pharmaceutically acceptable salts thereof are useful for treating cancer in humans, e.g., pediatric and geriatric populations, and in animals, e.g., veterinary applications. The disclosed methods can optionally include identifying a patient who is or can be in need of treatment of a cancer. Examples of cancer types treatable by the compounds and compositions described herein include bladder cancer, brain cancer, breast cancer, colorectal cancer, cervical cancer, gastrointestinal cancer, genitourinary cancer, head and neck cancer, lung cancer, ovarian cancer, pancreatic cancer, prostate cancer, renal cancer, skin cancer, and testicular cancer. Further examples include cancer and/or tumors of the anus, bile duct, bone, bone marrow, bowel (including colon and rectum), eye, gall bladder, kidney, mouth, larynx, esophagus, stomach, testis, cervix, mesothelioma, neuroendocrine, penis, skin, spinal cord, thyroid, vagina, vulva, uterus, liver, muscle, blood cells (including lymphocytes and other immune system cells). Further examples of cancers treatable by the compounds and compositions described herein include carcinomas, Karposi's sarcoma, melanoma, mesothelioma, soft tissue sarcoma, pancreatic cancer, lung cancer, leukemia (acute lymphoblastic, acute myeloid, chronic lymphocytic, chronic myeloid, and other), and lymphoma (Hodgkin's and non-Hodgkin's), and multiple myeloma.

The methods of treatment or prevention of cancer described herein can further include treatment with one or more additional agents (e.g., an anti-cancer agent or ionizing radiation). The one or more additional agents and the compounds and compositions or pharmaceutically acceptable salts thereof as described herein can be administered in any order, including simultaneous administration, as well as temporally spaced order of up to several days apart. The methods can also include more than a single administration of the one or more additional agents and/or the compounds and compositions or pharmaceutically acceptable salts thereof as described herein. The administration of the one or more additional agents and the compounds and compositions or pharmaceutically acceptable salts thereof as described herein can be by the same or different routes. When treating with one or more additional agents, the compounds and compositions or pharmaceutically acceptable salts thereof as described herein can be combined into a pharmaceutical composition that includes the one or more additional agents.

The methods and compounds as described herein are useful for both prophylactic and therapeutic treatment. As used herein the term treating or treatment includes prevention; delay in onset; diminution, eradication, or delay in exacerbation of signs or symptoms after onset; and prevention of relapse. For prophylactic use, a therapeutically effective amount of the compounds and compositions or pharmaceutically acceptable salts thereof as described herein are administered to a subject prior to onset (e.g., before obvious signs of cancer), during early onset (e.g., upon initial signs and symptoms of cancer), or after an established development of cancer. Prophylactic administration can occur for several days to years prior to the manifestation of symptoms of an infection. Prophylactic administration can be used, for example, in the chemopreventative treatment of subjects presenting precancerous lesions, those diagnosed with early stage malignancies, and for subgroups with susceptibilities (e.g., family, racial, and/or occupational) to particular cancers. Therapeutic treatment involves administering to a subject a therapeutically effective amount of the compounds and compositions or pharmaceutically acceptable salts thereof as described herein after cancer is diagnosed.

The disclosed subject matter also concerns methods for treating a subject having a metabolic disorder or condition. In one embodiment, an effective amount of one or more compounds or compositions disclosed herein is administered to a subject having a metabolic disorder and who is in need of treatment thereof. In some examples, the metabolic disorder can comprise type II diabetes. In some examples of the methods of treating of treating the metabolic disorder in a subject, the compound or composition administered to the subject can comprise a therapeutic moiety that can comprise a targeting moiety that can act as an inhibitor against PTP1B. In one particular example of this method the subject is obese and the method comprises treating the subject for obesity by administering a composition as disclosed herein.

The disclosed subject matter also concerns methods for treating a subject having an immune disorder or condition. In one embodiment, an effective amount of one or more compounds or compositions disclosed herein is administered to a subject having an immune disorder and who is in need of treatment thereof. In some examples of the methods of treating of treating the immune disorder in a subject, the compound or composition administered to the subject can comprise a therapeutic moiety that can comprise a targeting moiety that can act as an inhibitor against Pin1.

The disclosed subject matter also concerns methods for treating a subject having an inflammatory disorder or condition. In one embodiment, an effective amount of one or more compounds or compositions disclosed herein is administered to a subject having an inflammatory disorder and who is in need of treatment thereof.

The disclosed subject matter also concerns methods for treating a subject having cystic fibrosis. In one embodiment, an effective amount of one or more compounds or compositions disclosed herein is administered to a subject having cystic fibrosis and who is in need of treatment thereof. In some examples of the methods of treating the cystic fibrosis in a subject, the compound or composition administered to the subject can comprise a therapeutic moiety that can comprise a targeting moiety that can act as an inhibitor against CAL PDZ.

In some embodiments, the CPPs disclosed herein can be used for detecting or diagnosing a disease or condition in a subject. For example, a CPP can comprise a targeting moiety and/or a detectible moiety that can interact with a target, e.g., a tumor.

### Compositions, Formulations and Methods of Administration

In vivo application of the disclosed compounds, and compositions containing them, can be accomplished by any suitable method and technique presently or prospectively known to those skilled in the art. For example, the disclosed compounds can be formulated in a physiologically- or pharmaceutically-acceptable form and administered by any suitable route known in the art including, for example, oral, nasal, rectal, topical, and parenteral routes of administration. As used herein, the term parenteral includes subcutaneous, intradermal, intravenous, intramuscular, intraperitoneal, and intrasternal administration, such as by injection. Administration of the disclosed compounds or compositions can be a single administration, or at continuous or distinct intervals as can be readily determined by a person skilled in the art.

The compounds disclosed herein, and compositions comprising them, can also be administered utilizing liposome technology, slow release capsules, implantable pumps, and biodegradable containers. These delivery methods can, advantageously, provide a uniform dosage over an extended period of time. The compounds can also be administered in their salt derivative forms or crystalline forms.

The compounds disclosed herein can be formulated according to known methods for preparing pharmaceutically acceptable compositions. Formulations are described in detail in a number of sources which are well known and readily available to those skilled in the art. For example, Remington's Pharmaceutical Science by E. W. Martin (1995) describes formulations that can be used in connection with the disclosed methods. In general, the compounds disclosed herein can be formulated such that an effective amount of the compound is combined with a suitable carrier in order to facilitate effective administration of the compound. The compositions used can also be in a variety of forms. These include, for example, solid, semi-solid, and liquid dosage forms, such as tablets, pills, powders, liquid solutions or suspension, suppositories, injectable and infusible solutions, and sprays. The preferred form depends on the intended mode of administration and therapeutic application. The compositions also preferably include conventional pharmaceutically-acceptable carriers and diluents which are known to those skilled in the art. Examples of carriers or diluents for use with the compounds include ethanol, dimethyl sulfoxide, glycerol, alumina, starch, saline, and equivalent carriers and diluents. To provide for the administration of such dosages for the desired therapeutic treatment, compositions disclosed herein can advantageously comprise between about 0.1% and 100% by weight of the total of one or more of the subject compounds based on the weight of the total composition including carrier or diluent.

Formulations suitable for administration include, for example, aqueous sterile injection solutions, which can contain antioxidants, buffers, bacteriostats, and solutes that render the formulation isotonic with the blood of the intended recipient; and aqueous and nonaqueous sterile suspensions, which can include suspending agents and thickening agents. The formulations can be presented in unit-dose or multi-dose containers, for example sealed ampoules and vials, and can be stored in a freeze dried (lyophilized) condition requiring only the condition of the sterile liquid carrier, for example, water for injections, prior to use. Extemporaneous injection solutions and suspensions can be prepared from sterile powder, granules, tablets, etc. It should be understood that in addition to the ingredients particularly mentioned above, the compositions disclosed herein can include other agents conventional in the art having regard to the type of formulation in question.

Compounds disclosed herein, and compositions comprising them, can be delivered to a cell either through direct contact with the cell or via a carrier means. Carrier means for delivering compounds and compositions to cells are known in the art and include, for example, encapsulating the composition in a liposome moiety. Another means for delivery of compounds and compositions disclosed herein to a cell comprises attaching the compounds to a protein or nucleic acid that is targeted for delivery to the target cell. U.S. Pat. No. 6,960,648 and U.S. Application Publication Nos. 2003/0032594 and 2002/0120100 disclose amino acid sequences that can be coupled to another composition and that allows the composition to be translocated across biological membranes. U.S. Application Publication No. 200/20035243 also describes compositions for transporting biological moieties across cell membranes for intracellular delivery. Compounds can also be incorporated into polymers, examples of which include poly (D-L lactide-co-glycolide) polymer for intracranial tumors; poly[bis(p-carboxy phenoxy) propane; sebacic acid] in a 20:80 molar ratio (as used in GLIADEL); chondroitin; chitin; and chitosan.

For the treatment of oncological disorders, the compounds disclosed herein can be administered to a patient in need of treatment in combination with other antitumor or anticancer substances and/or with radiation and/or photodynamic therapy and/or with surgical treatment to remove a tumor. These other substances or treatments can be given at the same as or at different times from the compounds disclosed herein. For example, the compounds disclosed herein can be used in combination with mitotic inhibitors such as taxol or vinblastine, alkylating agents such as cyclophosamide or ifosfamide, antimetabolites such as 5-fluorouracil or hydroxyurea, DNA intercalators such as adriamycin or bleomycin, topoisomerase inhibitors such as etoposide or camptothecin, antiangiogenic agents such as angiostatin, antiestrogens such as tamoxifen, and/or other anti-cancer drugs or antibodies, such as, for example, GLEEVEC (Novartis Pharmaceuticals Corporation) and HERCEPTIN (Genentech, Inc.), respectively, or an immunotherapeutic such as ipilimumab and bortezomib.

In certain examples, compounds and compositions disclosed herein can be locally administered at one or more anatomical sites, such as sites of unwanted cell growth (such as a tumor site or benign skin growth, e.g., injected or topically applied to the tumor or skin growth), optionally in combination with a pharmaceutically acceptable carrier such as an inert diluent. Compounds and compositions disclosed herein can be systemically administered, such as intravenously or orally, optionally in combination with a pharmaceutically acceptable carrier such as an inert diluent, or an assimilable edible carrier for oral delivery. They can be enclosed in hard or soft shell gelatin capsules, can be compressed into tablets, or can be incorporated directly with the food of the patient's diet. For oral therapeutic administration, the active compound can be combined with one or more excipients and used in the form of ingestible tablets, buccal tablets, troches, capsules, elixirs, suspensions, syrups, wafers, aerosol sprays, and the like.

The disclosed compositions are bioavailable and can be delivered orally. Oral compositions can be tablets, troches, pills, capsules, and the like, and can also contain the following: binders such as gum tragacanth, acacia, corn starch or gelatin: excipients such as dicalcium phosphate: a disintegrating agent such as corn starch, potato starch, alginic acid and the like: a lubricant such as magnesium stearate; and a sweetening agent such as sucrose, fructose, lactose or aspartame or a flavoring agent such as peppermint, oil of wintergreen, or cherry flavoring can be added. When the unit dosage form is a capsule, it can contain, in addition to materials of the above type, a liquid carrier, such as a vegetable oil or a polyethylene glycol. Various other materials can be present as coatings or to otherwise modify the physical form of the solid unit dosage form. For instance, tablets, pills, or capsules can be coated with gelatin, wax, shellac, or sugar and the like. A syrup or elixir can contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound can be incorporated into sustained-release preparations and devices.

Compounds and compositions disclosed herein, including pharmaceutically acceptable salts or prodrugs thereof, can be administered intravenously, intramuscularly, or intraperitoneally by infusion or injection. Solutions of the active agent or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations can contain a preservative to prevent the growth of microorganisms.

The pharmaceutical dosage forms suitable for injection or infusion can include sterile aqueous solutions or dispersions or sterile powders comprising the active ingredient, which are adapted for the extemporaneous preparation of sterile injectable or infusible solutions or dispersions, optionally encapsulated in liposomes. The ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. Optionally, the prevention of the action of microorganisms can be brought about by various other antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the inclusion of agents that delay absorption, for example, aluminum monostearate and gelatin.

Sterile injectable solutions are prepared by incorporating a compound and/or agent disclosed herein in the required amount in the appropriate solvent with various other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, compounds and agents disclosed herein can be applied in as a liquid or solid. However, it will generally be desirable to administer them topically to the skin as compositions, in combination with a dermatologically acceptable carrier, which can be a solid or a liquid. Compounds and agents and compositions disclosed herein can be applied topically to a subject's skin to reduce the size (and can include complete removal) of malignant or benign growths, or to treat an infection site. Compounds and agents disclosed herein can be applied directly to the growth or infection site. Preferably, the compounds and agents are applied to the growth or infection site in a formulation such as an ointment, cream, lotion, solution, tincture, or the like.

Useful solid carriers include finely divided solids such as talc, clay, microcrystalline cellulose, silica, alumina and the like. Useful liquid carriers include water, alcohols or glycols or water-alcohol/glycol blends, in which the compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers, for example.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Useful dosages of the compounds and agents and pharmaceutical compositions disclosed herein can be determined by comparing their in vitro activity, and in vivo activity in animal models. Methods for the extrapolation of effective dosages in mice, and other animals, to humans are known to the art.

The dosage ranges for the administration of the compositions are those large enough to produce the desired effect in which the symptoms or disorder are affected. The dosage should not be so large as to cause adverse side effects, such as unwanted cross-reactions, anaphylactic reactions, and the like. Generally, the dosage will vary with the age, condition, sex and extent of the disease in the patient and can be determined by one of skill in the art. The dosage can be adjusted by the individual physician in the event of any counterindications. Dosage can vary, and can be administered in one or more dose administrations daily, for one or several days.

Also disclosed are pharmaceutical compositions that comprise a compound disclosed herein in combination with a pharmaceutically acceptable carrier. Pharmaceutical compositions adapted for oral, topical or parenteral administration, comprising an amount of a compound constitute a preferred aspect. The dose administered to a patient, particularly a human, should be sufficient to achieve a therapeutic response in the patient over a reasonable time frame, without lethal toxicity, and preferably causing no more than an acceptable level of side effects or morbidity. One skilled in the art will recognize that dosage will depend upon a variety of factors including the condition (health) of the subject, the body weight of the subject, kind of concurrent treatment, if any, frequency of treatment, therapeutic ratio, as well as the severity and stage of the pathological condition.

Also disclosed are kits that comprise a compound disclosed herein in one or more containers. The disclosed kits can optionally include pharmaceutically acceptable carriers and/or diluents. In one embodiment, a kit includes one or more other components, adjuncts, or adjuvants as described herein. In another embodiment, a kit includes one or more anti-cancer agents, such as those agents described herein. In one embodiment, a kit includes instructions or packaging materials that describe how to administer a compound or composition of the kit. Containers of the kit can be of any suitable material. e.g., glass, plastic, metal, etc., and of any suitable size, shape, or configuration. In one embodiment, a compound and/or agent disclosed herein is provided in the kit as a solid, such as a tablet, pill, or powder form. In another embodiment, a compound and/or agent disclosed herein is provided in the kit as a liquid or solution. In one embodiment, the kit comprises an ampoule or syringe containing a compound and/or agent disclosed herein in liquid or solution form.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. However, those of skill in the art should, in light of the present disclosure, appreciate that many changes can be made in the specific embodiments which are disclosed and still obtain a like or similar result without departing from the spirit and scope of the invention.

Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

Peptide Synthesis and Labeling. Linear peptides were synthesized by solid-phase peptide synthesis on Rink amide resin LS (0.49 mmol/g) using standard Fmoc chemistry, either manually or on a CEM Liberty Blue peptide synthesizer. The coupling reaction included 5 equivalents of Fmoc-amino acid, 5 equivalents of 2-(7-aza-1H-benzotriazole-1-yl)-1,1,3,3-tetramethyl-uronium hexafluorophosphate (HATU), and 10 equivalents of diisopropylethylamine (DIPEA). The N-terminal amine was deprotected by treatment with 20% piperidine in dimethylformamide (DMF) and acetylated by treatment with 10 equivalents acetic anhydride and 10 equivalents DIPEA in anhydrous dichloromethane (DCM) (2×15 min). CPP12 linear sequence was synthesized as described with an α-allyl-protected glutamate residue. The allyl protecting group was removed by treatment with 0.1 equivalent of Pd(PPh₃)₄ and 10 equivalents of phenylsilane in DCM (3×15 mins). The N-terminal Fmoc group was removed by piperidine and head-to-tail cyclization was performed by treatment with 5 equivalents of benzotriazole-1-yloxy-tris-pyrrolidino-phosphonium hexafluoro-phosphate (PyBOP), 5 equivalents of hydroxy benzotriazole, and 10 equivalents of DIPEA (2×90 min). Peptides were cleaved from resin by treatment with 91:3:3:3 (v/v) trifluoracetic acid (TFA)/H₂O/triisopropylsilane/ethanedithiol for 3 h. Cleaved peptide solution was concentrated by N₂ evaporation and triturated with cold ether (3×). Peptides were purified by reversed-phase HPLC on a C₁₈ column eluted with 0.05% TFA H₂O and acetonitrile. Peptide cyclization was carried out by incubation with 1.2 equivalents of BiBr₃ (as a saturated 60 mM stock in DMF) and 2.5 equivalents of tris(2-carboxyethyl)phosphine (TCEP) in a TRIS buffer (pH 7.4). Excess insoluble bismuth was pelleted by centrifugation. Cyclic peptides were purified by HPLC. Fluorescent labeling was achieved by incubating cyclic peptides with 1.1 eq of N-hydroxysuccinimide ester activated fluorophore in a NaHCO₃ solution (pH 8) for 90 min. Labeled peptides was purified by HPLC. Purity and identity of product (>95%) was confirmed at each stage by ultra-performance liquid chromatography in tandem with an electrospray ionization mass spectrometer (UPLC-MS).

Cell culture. HeLa cells were maintained in Dulbecco's modified eagle media (DMEM) supplemented with 10% fetal bovine serum (FBS) and 1% penicillin/streptomycin. Cells were cultured in a humidified incubator at 37° C. with 5% CO₂.

Flow cytometry. HeLa cells were cultured in 12-well plates (1.5×10⁵ cells/well) overnight. The following day, cells were incubated with 5 μM fluorescently labeled peptide for 2 h in DMEM supplemented with 1% FBS and 1% antibiotic solution. DMSO was used as a negative control. After incubation, cells were detached from plate with 350 μL of 0.25% Trypsin-EDTA solution, diluted with Dulbecco's phosphate buffered saline (DPBS), and pelleted at 300 g for 5 min. Cells were washed twice with DPBS, resuspended in DPBS, and analyzed on a BD FACS LSR II flow cytometer.

Confocal microscopy. HeLa cells (5×10⁵ cells/mL in 300 μL volume) were cultured overnight in DMEM (10% FBS, 1% antibiotic solution) in a Grenier Bio-One 35/10 mm glass-bottomed microwell dish with four compartments. The following day, cells were incubated with 5 μM fluorescently labeled peptide for 2 h in phenol-red free DMEM (1% FBS, 1% antibiotic solution) and washed with warm media prior to imaging. Cells were imaged on a Nikon A1R live-cell confocal laser scanning microscope (ECLIPSE Ti-E automated, inverted) equipped with a 100× oil objective (1.45 N.A.) and a heated chamber (37° C.) supplied with 5% CO₂. TMR-labeled peptides were imaged by excitation with a λ_{Ex} of 561 nm. Differential interference contrast (DIC) images were obtained to validate the morphology of cells.

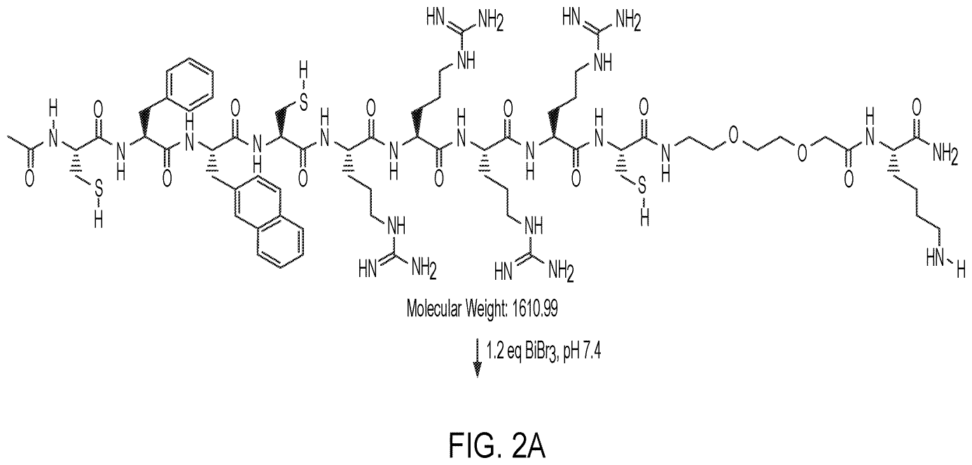

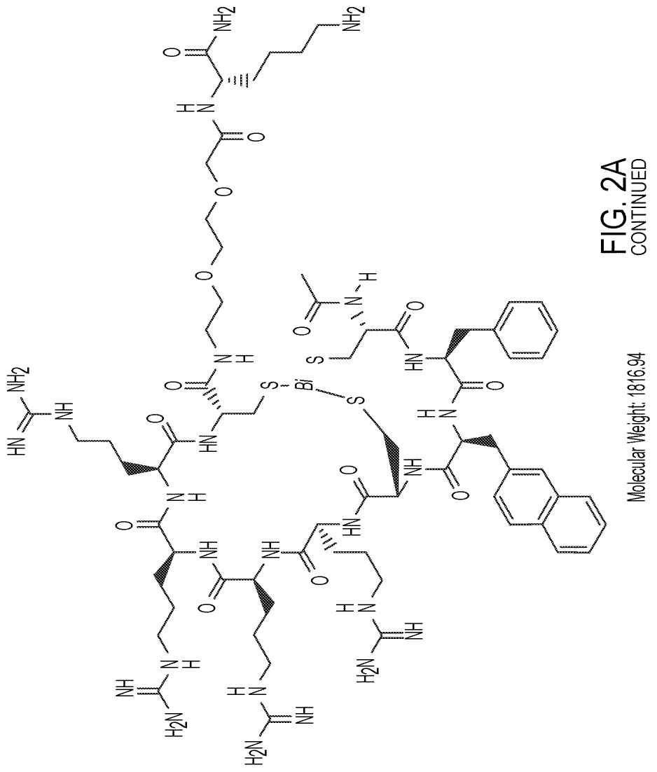

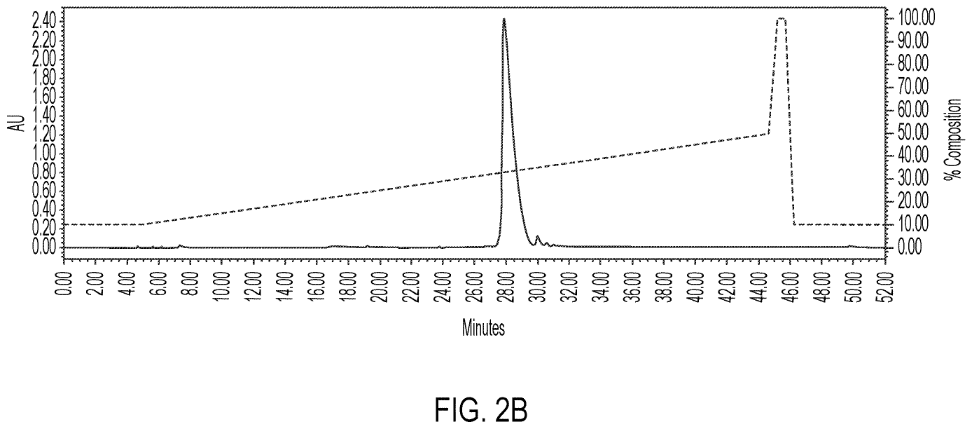

Results. A library of bismuth-cyclized peptides (BCPs, Tables 4-7) was designed, synthesized, and tested for entry into Hela cells. The BCPs are separated into two different families-those that contain of only proteinogenic amino acids (designated as “pBCPs”, where “p” stands for “proteinogenic”: Tables 4 and 6) and those that contain both proteinogenic and non-proteinogenic amino acids (BCPs: Tables 5 and 7). pBCPs are intended for future incorporation into recombinant proteins, although they can also be chemically synthesized and used to deliver diverse cargoes other than peptides and proteins. BCPs must be chemically synthesized and subsequently conjugated to a cargo of interest, including peptides and proteins. The peptides were synthesized on Rink amide resin by the standard Fmoc/HATU solid-phase peptide synthesis (SPPS) protocol. The peptides were purified by reversed-phase HPLC and reacted with 1.2 equivalents of BiBr₃ in an aqueous buffer (Tris, pH 7.4), in the presence of 2.5 equivalents of tris(2-carboxyethyl)phosphine (TCEP). Insoluble excess bismuth was removed by centrifugation. HPLC analysis revealed a single peak corresponding to the cyclized peptide, while no peak corresponding to the linear peptide precursor was observed, indicating quantitative conversion of the linear peptide into the desired cyclic form (FIGS. 2A and 2B). It was also found that the crude peptide (after trituration with diethylether) can be cyclized with BiBr3 prior to HPLC purification.

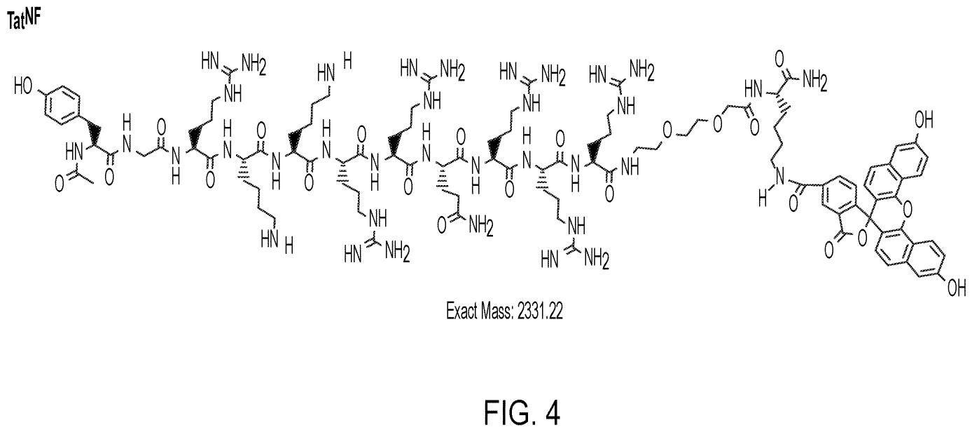

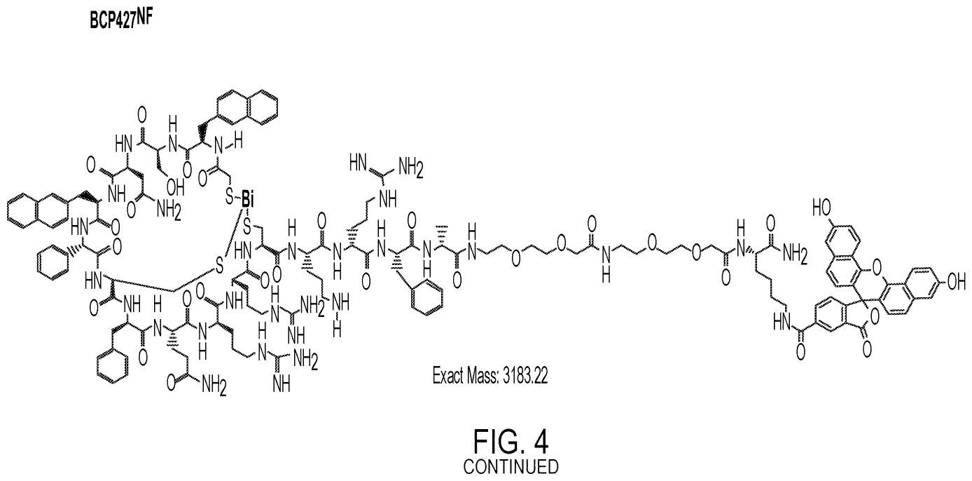

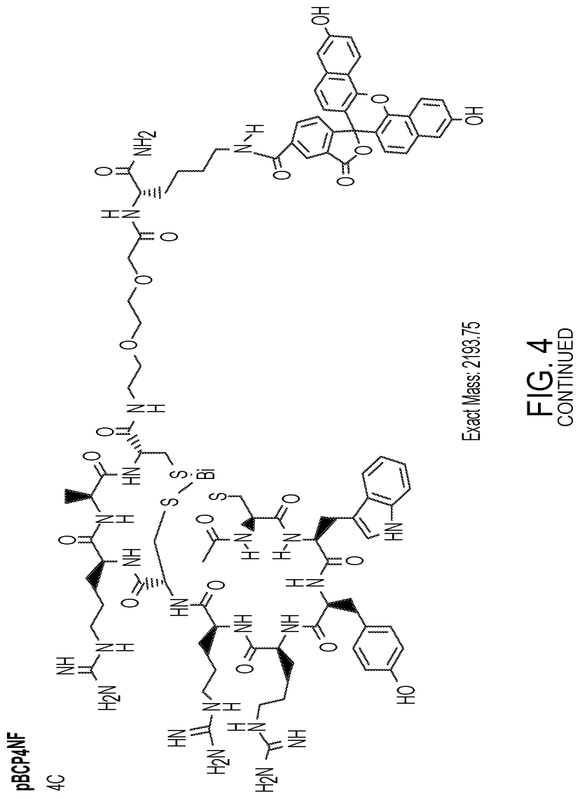

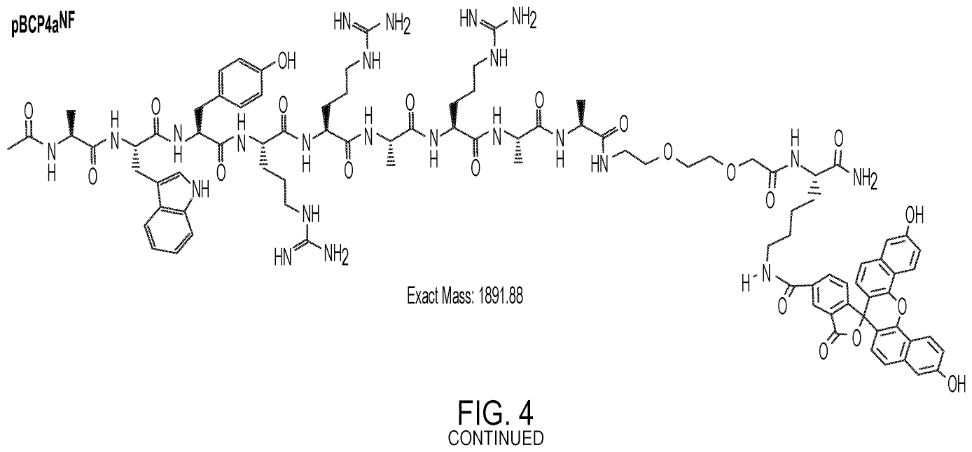

To evaluate their cell entry efficiencies, the BCPs and pBCPs were labeled with tetramethylrhodamine (TMR) or naphthofluorescein (NF) on a C-terminal lysine residue (FIGS. 2A, 2B, and 4). TMR- and NF-labeled CPP12 (one of the most active CPPs known to date) and Tat (a prototypical first-generation CPP that is widely used in research and drug delivery) were also synthesized as benchmarks. The labeled peptides were purified again by reversed-phase HPLC and their authenticity was confirmed by high-resolution mass spectrometry.

HeLa cells were treated with 5 μM dye-labeled BCPs (or pBCPs), CPP12, Tat, or DMSO (equal volume) for 2 h at 37° C., washed to remove any peptides in the medium, and analyzed by flow cytometry. The mean fluorescence intensity (MFI) value of TMR (which is pH insensitive) reflects the total amount of CPP inside the HeLa cells (total cellular uptake). On the other hand, NF is a pH sensitive dye, having an apparent pKₐ of 7.8. NF is unprotonated and fluorescent inside the neutral environment of the cytosol (and nucleus) but is nearly completely protonated and nonfluorescent in the acidic environments of endosomes and lysosomes. Thus, the MFI value of NF provides a convenient estimate of the peptides that have reached the cytosol.

Most of the pBCPs have both total cellular uptake and cytosolic entry efficiencies that are 10-15% of that of CPP12 (Table 4). The most active CPP of this series is pBCP10 (CWYWRRCRAC); with a cytosolic entry efficiency of 17% (relative to CPP12), it is 6-fold less active than CPP12 but 5-fold more active than Tat (3.3%). To assess the effect of bismuth-mediated cyclization on CPP activity, we generated a linear variant of bBCP4, in which the three cysteine residues were replaced with Ala (pBCP4a). The linear peptide (pBCP4a) is ˜2-fold less active than pBCP4, demonstrating the benefit of peptide cyclization for cell entry. Note that the effect of cyclization observed here (2-fold) is much smaller than some of the previous observations (23-fold) (Qian, Z., et al. (2013) Efficient delivery of cyclic peptides into mammalian cells with short sequence motifs ACS Chem. Biol. 8, 423-431), suggesting that in the case of pBCP4, the cyclized peptide may not adopt the optimal conformation for membrane binding.

TABLE 4
Sequences and Cellular Entry Efficiencies of Bismuth CPPs Containing
Proteinogenic Residues Only (Genetically Encodable)

| | | | MFI^{TMR} | No. of | | No. of |
|---|---|---|---|---|---|---|
| | | | (Total | Rep- | MFI^{NF} | Rep- |
| SEQ ID | | | Cellular | licates | (Cytosolic | licates |
| NO. | CPP | Sequenceª | Uptake)^{b} | (n) | Entry)^{b} | (n) |
| 10 | CPP12 | cyclo(FfΦRrRrQ) | 100 | | 100 | |
| 11 | Tat | YGRKKRRQRRR | | | 3.3 ± 0.3 | 3 |
| 12 | pBCP1 | CRRRRCWYWC | 9.3 ± 1.9 | 3 | 12 ± 4 | 3 |
| 13 | pBCP2 | CRRCRRWYWC | N/a* | 0 | | |
| 14 | pBCP3 | CARCRRRWYWC | 3 | 1 | | |
| 15 | pBCP4 | CWYRRCRAC | 11 ± 1 | 3 | 10 ± 4 | 3 |
| 16 | pBCP4a | AWYRRARAA | | | 5.4 | 1 |
| 19 | pBCP5 | CARCRRYWC | 9.2 | 1 | | |
| 17 | pBCP6 | CFWRCRRC | 9.0 ± 0.7 | 2 | | |
| 19 | pBCP7 | CRRCRWFC | 10 ± 0.1 | 2 | | |
| 20 | pBCP8 | CRRRWYWCAAC | 8.4 | 1 | | |
| 21 | pBCP9 | CWYRRRCRAC | | | 12 ± 4 | 3 |
| 22 | pBCP10 | CWYWRRCRAC | | | 17 ± 2 | 3 |
| 23 | pBCP11 | CWYCRRRC | | | 14 ± 1 | 3 |
| 24 | pBCP12 | CWYRRRCAC | | | 12 ± 1 | 3 |
| 25 | pBCP13 | CWYRCRRC | | | 11 ± 1 | 3 |
| 26 | pBCP15 | CYYRRCRAC | | | 16 ± 3 | 3 |
| 27 | pBCP16 | CFFRRRCRAC | | | 15 ± 1 | 3 |
| 28 | pBCP17 | CFFFRRRCRAC | | | 15 ± 1 | 3 |
| 29 | pBCP427 | CWSNWFCFQRRCRRFA | | | 41 ± 5 | 3 |

^{a}All peptides contained an N-terminal acetyl group and a C-terminal miniPEG-Lys(dye)-NH₂. Φ, L-2-naphthylalanine; f, D-phenylalanine; r, D-arginine.
^{b}All values are relative to that of CPP12 (100%).

Incorporation of non-proteinogenic amino acids such as Nal renders BCPs generally more active than pBCPs (Tables 4 and 5), in agreement with earlier findings (Id.). Also as previously observed (Qian Z, et al. Discovery and Mechanism of Highly Efficient Cyclic Cell-Penetrating Peptides. Biochemistry. 2016; 55(18), 2601-2612), altering the stereochemistry of amino acids significantly increases the cell entry efficiency, relative to BCPs of all-L configurations (e.g., compare BCP3 vs BCP8 and BCP10). However, the specific stereochemical pattern (i.e., the actual peptide sequence) required for high cellular entry efficiency appears to be different from that of previous cyclic CPPs and remains unpredictable. For example, the addition of a Phe to CPP9 [cyclo(fΦRrRrQ)] gives CPP12 [cyclo(FfΦRrRrQ)] and increases the cytosolic entry efficiency by 2-fold (Qian. Z, et al. Discovery and Mechanism of Highly Efficient Cyclic Cell-Penetrating Peptides. Biochemistry. 2016; 55(18), 2601-2612.), but the same addition to BCP4 caused a small decrease in the cytosolic entry efficiency (Table 5, compare BCP4 and BCP5). This is not entirely unexpected, as bicyclization by bismuth and head-to-tail cyclization likely constrain a peptide sequence into different preferred conformations. Importantly, BCP427, which is an analog of a highly cell-permeable Ras inhibitor B4-27 (Buyanova, M., (2021) Discovery of a Bicyclic Peptidyl Pan-Ras Inhibitor. Journal of medicinal chemistry, 64(17), 13038-13053), has a cytosolic entry efficiency rivaling that of CPP12. pBCP427, which is also an analog of B4-27 but consists of only proteinogenic amino acids, has a cytosolic entry efficiency 41% of CPP12 (Table 4).

TABLE 5
Sequences and Cellular Entry Efficiencies of Bismuth CPPs Containing Non-
proteinogenic Residues

| | | | MFI^{TMR} | No. of | | No. of |
|---|---|---|---|---|---|---|
| | | | (Total | Rep- | MFI^{NF} | Rep- |
| SEQ ID | | | Cellular | licates | (Cytosolic | licates |
| NO. | CPP | Sequence^{a} | Uptake)^{b} | (n) | Entry)^{b} | (n) |
| 10 | CPP12 | cyclo(FfΦRrRrQ) | 100 | | 100 | |
| 11 | Tat | YGRKKRRQRRR | | | 3.3 ± 0.3 | 3 |
| 30 | BCP1 | Ac-CFΦCRRRRC | 12 ± 2 | 2 | | |
| 31 | BCP2 | Ac-CRRRRCΦFC | 15 ± 4 | 2 | | |
| 32 | BCP3 | Ac-CFΦRRCRRC | 13 ± 2 | 2 | | |
| 33 | BCP4 | Ac-CrRrRCΦfC | | | 51 ± 16 | 4 |
| 34 | BCP5 | Ac-CrRrRCΦfFC | | | 45 ± 1 | 3 |
| 35 | BCP8 | Ac-CFΦRRCrrC | | | 38 ± 1 | 3 |
| 36 | BCP9 | Ac-CΣΦRRCrrC | | | 43 | 1 |
| 37 | BCP10 | Ac-CfΦRrCRrC | | | 56 ± 32 | 2 |
| 38 | BCP11 | Ac- | | | 13 | 1 |
| | | CFΦRRRRCAAAC | | | | |
| 39 | BCP13 | Ac-CFΦRrRCAAC | | | 30 ± 3 | 3 |
| 40 | BCP427 | HS-CH₂-CO- | | | 100 ± 13 | 2 |
| | | φSNφΠCfQrRCOrFa | | | | |

^{a}All peptides contained a C-terminal miniPEG-Lys(dye)-NH₂. Φ, L-2-naphthylalanine; Σ, 3-(3-benzothienyl)-L-alanine; f, D-phenylalanine; r, D-arginine; φ, D-2-naphthylalanine; Π, L-phenylglycine; O, L-ornithine; a, D-alanine.
^{b}All values are relative to that of CPP12 (100%).

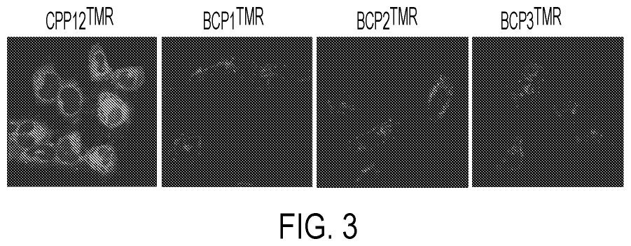

The cellular entry of BCP1-3 was further confirmed by live-cell confocal microscopy. HeLa cells were incubated with 5 μM TMR-labeled peptide for 2 h in the presence of 1% FBS in media. The cells were washed twice with fresh media and imaged without fixation. All three peptides showed readily visible TMR fluorescence in the cytoplasmic region of the cells, although the fluorescence pattern was predominantly punctate, suggesting that a significant fraction of the internalized BCPs were still entrapped inside the endosomes and lysosomes (FIG. 3).

The above results demonstrate that linear peptide containing arginines, hydrophobic residues, and three imbedded cysteine residues can be readily converted into bicyclic CPPs by the addition of a Bi³⁺ ion. The resulting CPPs enter the cytosol of HeLa cells with efficiencies approaching that of CPP12, one of the most potent CPPs known to date. The cyclization strategy should be readily applicable to recombinant proteins. The cellular entry efficiency of the BCPs and pBCPs can be further improved by optimizing the peptide sequences and/or stereochemistry.

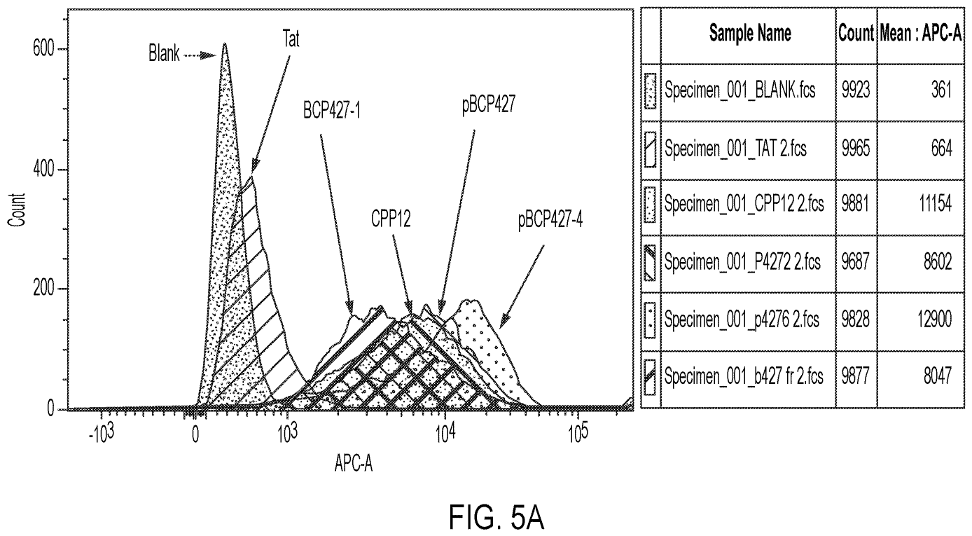

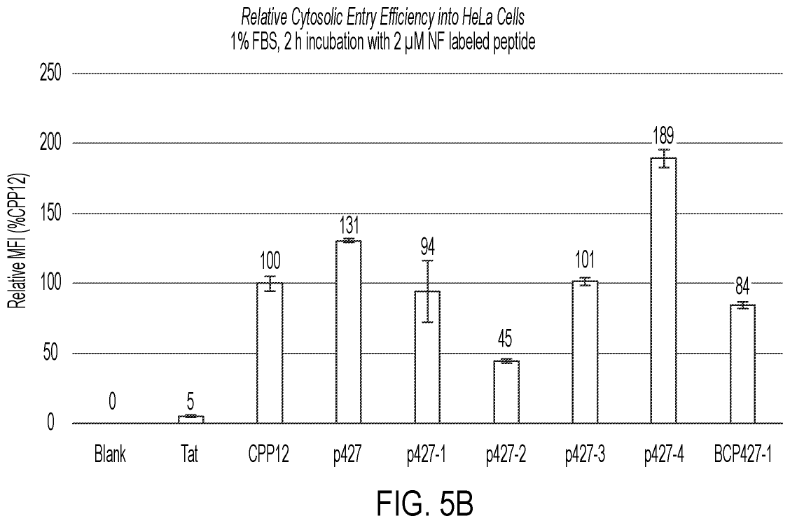

An additional set of sequences were prepared starting from pBCP427. The C-terminal exocyclic residues, which are expected to proteolytically labile, were truncated. Interestingly, while removal of the C-terminal Ala and Phe progressively reduced the cytosolic entry efficiency, further truncation from the C-terminus increases the cell entry (FIGS. 5A and 5B, and Table 6). Most notably, pBCP427-4, which contains no exocyclic residue, has a cytosolic entry efficiency of 189% (relative to CPP12 which is defined as 100%). Another important feature of pBCP427-4 is that it contains only two arginine residues. Polyarginines bind to proteoglycans on mammalian cell surface and may adversely affect their biodistribution (Qian, Z., et al., (2014) Early endosomal escape of a cyclic cell-penetrating peptide allows effective cytosolic cargo delivery. Biochemistry 53:4034-4046). High cationic charges, especially when in combination with hydrophobic moieties, increases the probability of mast cell degranulation (Lorenz, D., et al., (1998) Mechanism of peptide-induced mast cell degranulation. Translocation and patch-clamp studies. J. General Physiol. 112 (5), 577-591).

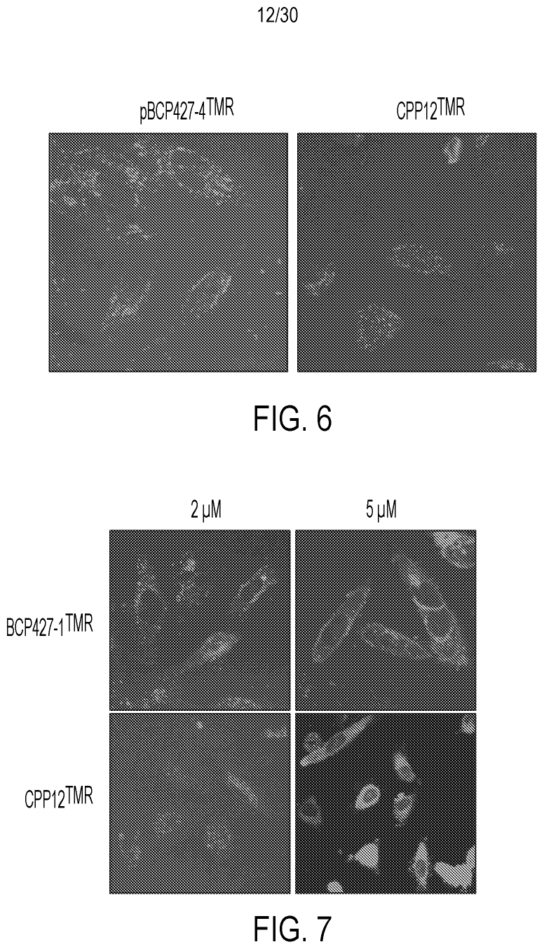

Note that cell entry assays were performed by treating HeLa cell with 2 μM naphthofluorescein (NF)-labeled peptide for 2 h at 37° C. Under this condition, the BCPs enter the cell almost exclusively by endocytosis and endosomal escape. A small fraction of the CPP12^{NF}-treated cells (˜10%) underwent direct translocation of CPP12^{NF}; this fraction of highly fluorescent cells was not included in the data analysis. The cellular entry of pBCP427-4 was confirmed by live-cell confocal microscopy (FIG. 6).

TABLE 6
Sequences and Cytosolic Entry Efficiencies of Bismuth CPPs Containing
Proteinogenic Residues Only (Genetically Encodable)

| | | | | No. of |
|---|---|---|---|---|
| SEQ ID | | | | Replicates |
| NO. | CPP | Sequenceª | MFI^{b} | (n) |
| 10 | CPP12 | cyclo(FfΦRrRrQ) | 100 | 3 |
| 11 | Tat | Ac-YGRKKRRQRRR | 5.0 ± 0.6 | 3 |
| 29 | pBCP427 | Ac-CWSNWFCFQRRCRRFA | 131 ± 1 | 3 |
| 41 | pBCP427-1 | Ac-CWSNWFCFQRRCRRF | 94 ± 22 | 3 |
| 42 | pBCP427-2 | Ac-CWSNWFCFQRRCRR | 45 ± 1 | 3 |
| 43 | pBCP427-3 | Ac-CWSNWFCFQRRCR | 101 ± 3 | 3 |
| 44 | pBCP427-4 | Ac-CWSNWFCFQRRC | 189 ± 6 | 3 |
| 45 | pBCP427-5 | Ac-CWSNWCFQRRC | 76 ± 1 | 3 |
| 46 | pBCP427-6 | Ac-CWSNWFCQRRC | 65 ± 1 | 3 |
| 47 | pBCP427-7 | Ac-CWSQWFCFQRRC | 69 ± 3 | 3 |
| 48 | pBCP427-8 | Ac-CWQQWCFQRRC | | |
| 49 | pBCP427-9 | Ac-CWQWFCFQRRC | | |
| 50 | pBCP427-10 | Ac-CFSNWFCFQRRC | | |
| 51 | pBCP427-11 | Ac-CWSNFFCFQRRC | | |
| 52 | pBCP427-12 | Ac-CWSNWYCFQRRC | | |
| 53 | pBCP427-13 | Ac-CWSNWFCYQRRC | | |
| 54 | pBCP427-14 | Ac-CWNNWFCFQRRC | | |
| 55 | pBCP427-15 | Ac-CWSNWFCFRRRC | | |

^{a}All peptides contained an N-terminal acetyl group and a C-terminal miniPEG-Lys(NF)-NH₂. Φ, L-2-naphthylalanine; f, D-phenylalanine; r, D-arginine; and NF, naphthofluorescein.
^{b}All values were determined at 2 μM peptide and are relative to that of CPP12 (100%).

For BCPs containing non-proteinogenic amino acids, we found that C-terminal truncation of BCP427-1 also increased the cytosolic entry by ˜50% (Table 7 and FIG. 3), although they are less active than pBCP427-4. In addition, we converted a previously reported monocyclic peptidyl Ras inhibitor, 9A5 [3], into BCPs through bismuth-mediated bicyclization (Table 7). The resulting peptides, BCP9A5-1 and BCP9A5-2, showed respectable cytosolic entry efficiencies (30-43% of CPP12). Increasing the size and/or the number of hydrophobic residues of BCP4 and BCP5 resulted in greatly improved and highly active CPPs (Table 7, 86-444% cytosolic entry efficiencies for BCP14-16). Additional SAR and further improvement of the BCPs are ongoing (Table 7).

TABLE 7
Sequences and Cytosolic Entry Efficiencies of Bismuth CPPs Containing Non-
proteinogenic Residues

| | | | MFI^{NF} | No. of |
|---|---|---|---|---|
| SEQ ID | | | (Cytosolic | Replicates |
| NO. | CPP | Sequence^{a} | Entry)^{b} | (n) |
| 10 | CPP12 | cyclo(FfΦRrRrQ) | 100 | 3 |
| 11 | Tat | Ac-YGRKKRRQRRR | 5.0 ± 0.6 | 3 |
| 56 | BCP14 | HS-CH₂-CO-rRrRCΦφC | 86 ± 2 | 2 |
| 57 | BCP15 | HS-CH₂-CO-rRrRC(Bta)φC | 108 ± 4 | 2 |
| 58 | BCP16 | HS-CH₂-CO-rRrRCΦ(Bta)φC | 444 ± 2 | 2 |
| 59 | BCP17 | HS-CH₂-CO-rRrRCΦC | | |
| 40 | BCP427 | HS-CH₂-CO- | 100 ± 13** | 2 |
| | | φSNφΠCfQrRCOrFa | | |
| 60 | BCP427-1 | HS-CH₂-CO- | 84 ± 2 | 3 |
| | | φSNφFCfQrRCRrFa | | |
| 61 | BCP427-2 | HS-CH₂-CO-φSNφFCfQrRC | 118 ± 4 | 3 |
| 62 | BCP427-3 | Ac-CΦSNWFCFQRRC | | |
| 63 | BCP427-4 | Ac-C(Bta)SNWFCFQRRC | | |
| 64 | BCP427-5 | Ac-CφSNWFCFQRRC | | |
| 65 | BCP427-6 | Ac-CWSNΦFCFQRRC | | |
| 66 | BCP427-7 | Ac-CWSN(Bta)FCFQRRC | | |
| 67 | BCP427-8 | Ac-CWSNWφCFQRRC | | |
| 68 | BCP427-9 | Ac-CWSNWFCfQRRC | | |
| 69 | BCP427-10 | Ac-CWSNWFCFQrRC | | |
| 70 | BCP427-11 | Ac-CWSNWFCFQRrC | | |
| 71 | BCP427-12 | Ac-CΦSNΦFCFQRRC | | |
| 72 | BCP427-13 | Ac-CφSNφFCFQRRC | | |
| 73 | BCP9A5-1 | Ac-CRRRφRFCQWC | 30 ± 1 | 3 |
| 74 | BCP9A5-2 | Ac-CRRRφCF(dNle)QWC | 43 ± 2 | 3 |

^{a}All peptides contained a C-terminal miniPEG-Lys(dye)-NH₂. Φ, L-2-naphthylalanine; Σ, 3-(3-benzothienyl)-L-alanine; f, D-phenylalanine; r, D-arginine; φ, D-2-naphthylalanine; Π, L-phenylglycine; O, L-ornithine; a, D-alanine; dNle, D-norleucine.
^{b}All values were determined at 2 μM peptide and are relative to that of CPP12 (100%).
**Value determined at 5 μM peptide.

Proteolytic Stability of BCP4, BCP16, and pBCP427-4. BCPs (100 μM) were incubated in 25% human serum diluted in phosphate-buffered saline for 24 h at 37° C. One hundred-μL aliquots were withdrawn from the mixture at various time points and mixed with 200 μL of 1:1 (v/v) 15% trichloroacetic acid in methanol and acetonitrile. The samples were centrifuged for 5 min in a microcentrifuge and stored at 4° C. until analysis by UPLC-MS equipped with a C-18 column. The column was eluted with a linear gradient of 5-100% acetonitrile in water containing 0.1% TFA over 7.5 min and the eluant was monitored at 214 nm. The area underneath the peak for any remaining BCP was integrated and compared to that at time 0 and the percentage of remaining BCP was plotted as a function of the incubation time. When integration of the UV absorbance was not possible due to overlapping of the peptide signal with that of serum proteins, the signal abundance from the mass spectrum was used to estimate the % peptide remaining.

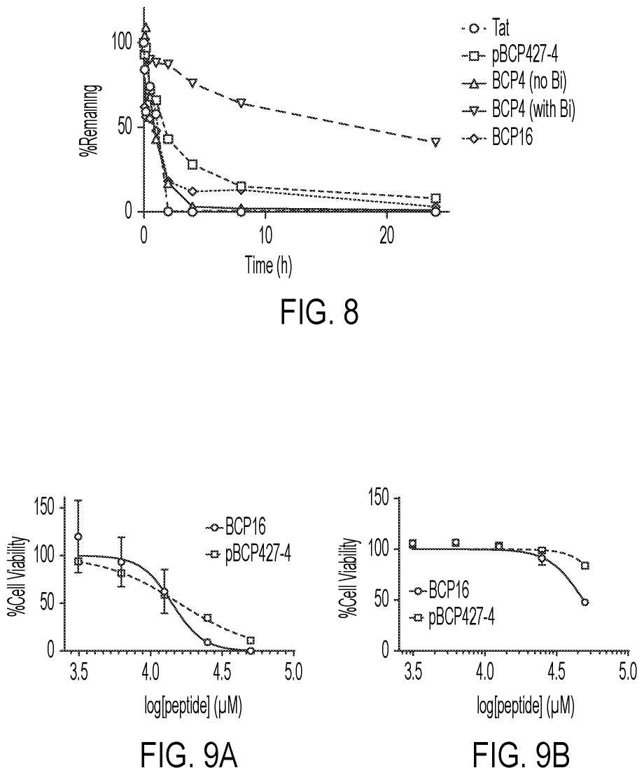

BCP16 and pBCP427-4 are unstable in human serum, having tin values of 1.1 and ˜2 h, respectively (FIG. 8). On the other hand, BCP4 is more stable, showing a tin value of ˜18 h: ˜40% of BCP4 remained intact even after 24 h of incubation. The linear form of BCP4 (without bismuth) is much less stable than the cyclized form (t_{1/2}<1 h), demonstrating that bismuth-mediated bicyclization results in substantial stabilization of the peptides against proteolysis.

Cytotoxicity of BCPs. The cytotoxicity of pBCP427-4 and BCP16 was tested 20) against HeLa and HEK293T cell lines by using Promega's Cell-Glo assay. The cells were seeded in a 96-well plate (5000 cells/well) in 100 μL of DMEM supplemented with 10% FBS and 1% penicillin/streptomycin. The following day. 10 μL of peptide of various concentrations was added to the cells. A row of wells was treated with DMSO as a control. After 72 h, all wells were treated with an equal volume (110 μL) of Promega CellTiter-Glo 2.0 assay solution and shaken for 30 min. Luminescent signal was measured on a Tecan Infinite Pro plate reader. Data was analyzed via GraphPad Prism to calculate the concentration at which 50% of cells are nonviable (IC₅₀). pBCP427-4 showed IC₅₀ values of 16 μM against HEK293T cells and >50 μM against Hela cells (FIGS. 9A-9B). BCP16 had IC₅₀ values of 14 μM against HEK293T cells and 49 μM against HeLa cells.

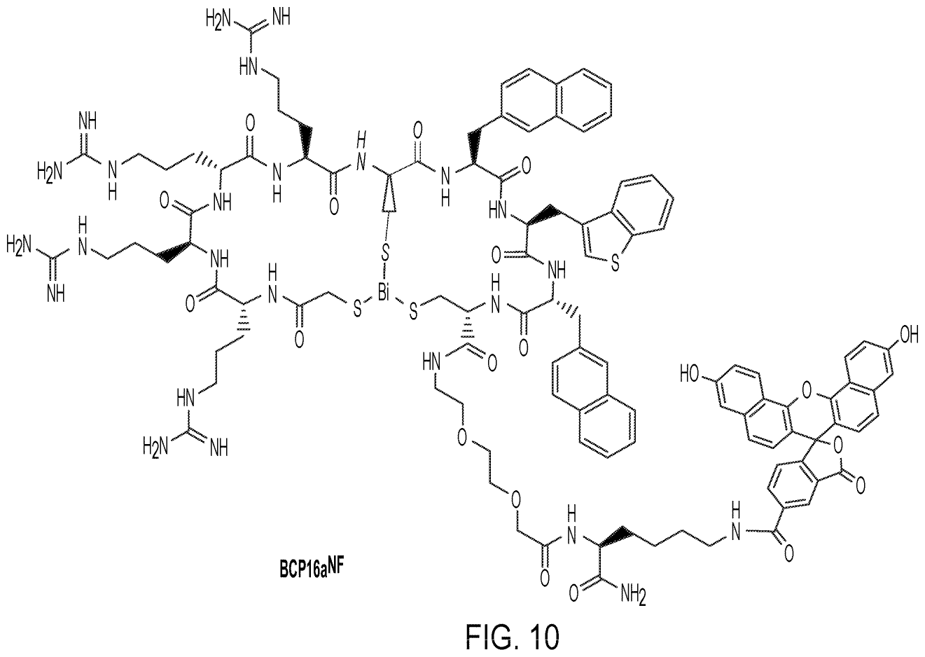

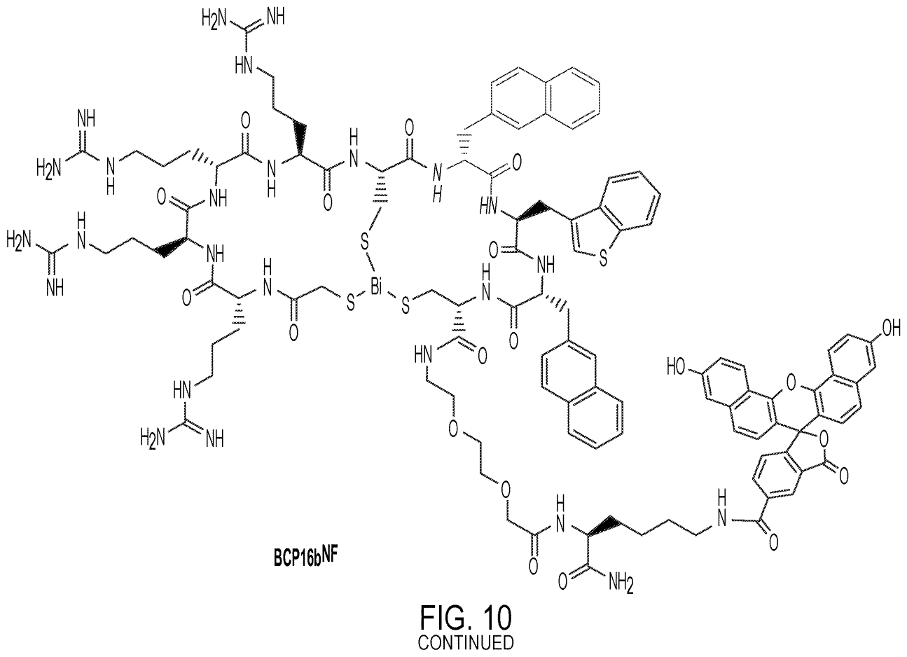

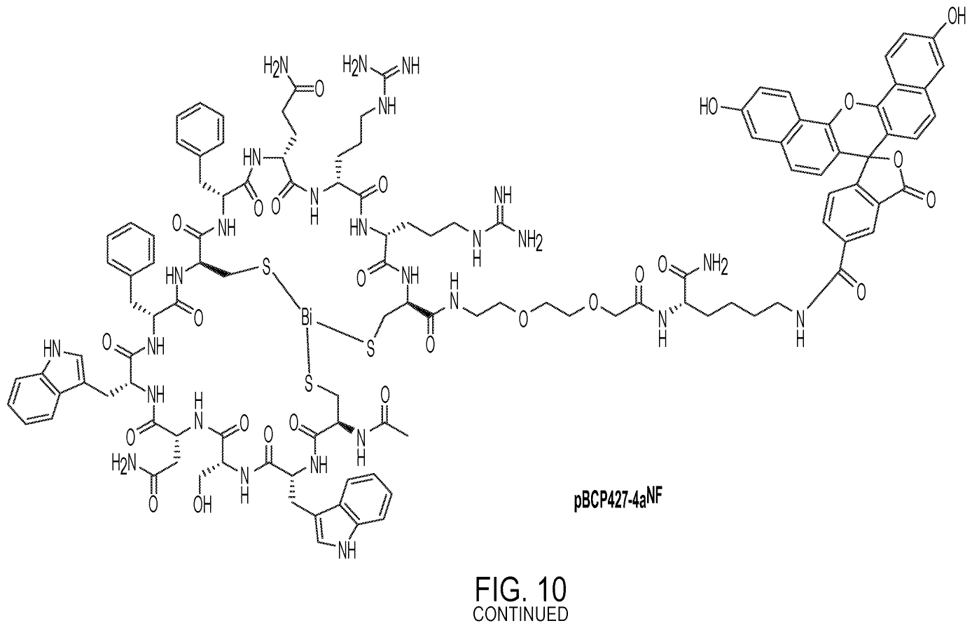

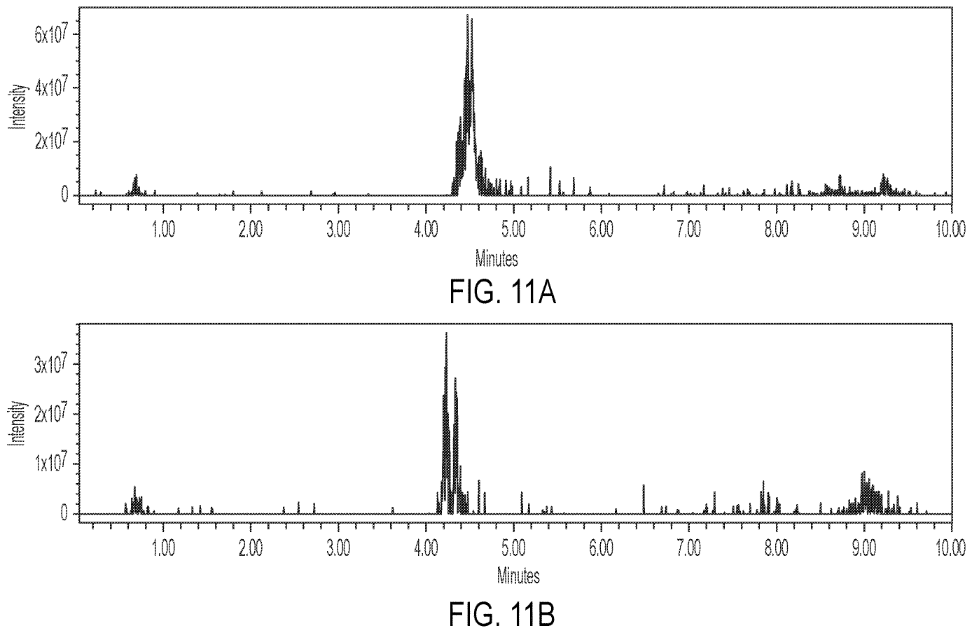

BCP16 and pBCP427-4 Analogs of Improved Proteolytic Stability. Although Bi³⁺ ions bind to peptides containing three adjacent cysteines with very high thermodynamic stability (Potocki, S., et al . . . (2011). Metal binding ability of cysteine-rich peptide domain of ZIP13 Zn²⁺ ions transporter. Inorganic Chemistry, 50 (13), 6135-61451; Voss, S., et al., (2022). Peptide-Bismuth Bicycles: In Situ Access to Stable Constrained Peptides with Superior Bioactivity. Angewandte Chemie (International ed. in English), 61(4), e2021138572), the Bi³⁺/peptide complex is kinetically labile and in rapid equilibrium with the free peptide (Sadler P J, et al., (1996) Bismuth (III) complexes of the tripeptide glutathione (γ-L-Glu-L-Cys-Gly). Chemistry 2 (6), 701-7083). Presumably, the transient linear peptide form is recognized and degraded by serum proteases. To generate BCPs of greater proteolytic stability, the enantiomer of pBCP427-4, pBCP427-4a (which contains all D-amino acids), was synthesized (Table 8 and FIG. 10). To improve the proteolytic stability of BCP16, the stereochemistry of the internal cysteine or L-naphthylalanine was inverted to produce BCP16a and BCP16b, respectively (FIG. 10). These substitutions remove an all-L tetrapeptide motif in BCP16, Arg-Cys-Nal-Bta, which is likely a hot spot for proteolytic degradation. Satisfyingly, the three BCP analogs showed substantially improved serum stability; preliminary studies indicate that BCP16a and BCP16b have serum tin values of ˜24 h (FIGS. 11A-11B), while pBCP427-4a showed no significant degradation after 24 h of incubation in human serum (t_{1/2}>24 h).

BCP16a, BCP16b, and pBCP427-4a were labeled with NF and their cytosolic entry efficiencies were assessed by flow cytometry analysis of HeLa cells after treatment with 2 μM peptide at 37° C. for 2 h. In the presence of 1% FBS, BCP16a, BCP16b, and pBCP427-4a exhibited cytosolic entry efficiencies of 156%, 98%, and 33%, respectively, relative to that of CPP12 (Table 8). Interestingly, in the presence of 10% FBS (and under otherwise identical conditions), BCP16a, BCP16b, and pBCP427-4a showed cytosolic entry efficiencies of 821%, 169%, and 85%, respectively, relative to that of CPP12 (data not shown). CPP12 was previously shown to be highly sensitive to serum proteins (Buyanova, M., et al., (2022). Discovery of a Cyclic Cell-Penetrating Peptide with Improved Endosomal Escape and Cytosolic Delivery Efficiency. Molecular pharmaceuties, 19 (5). 1378-1388). The above data suggest that BCP16a, BCP16b, and pBCP427-4a are less sensitive to serum proteins (as compared to CPP12) and remain effective in human serum.

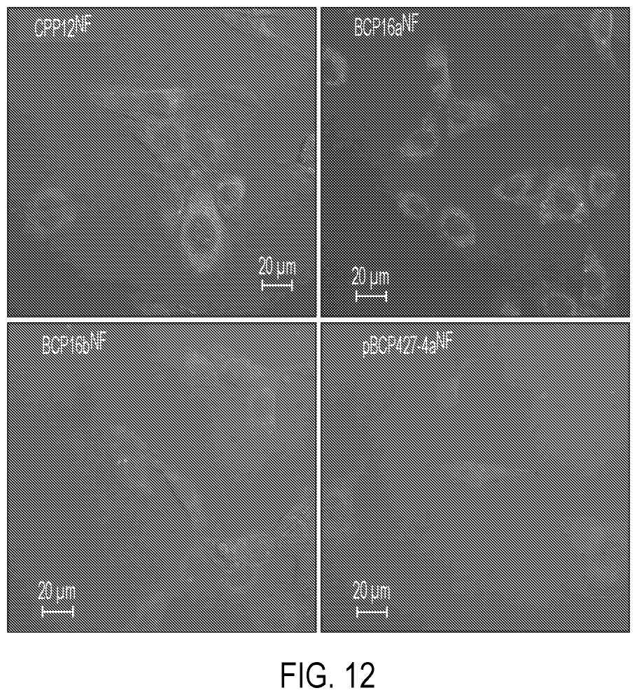

The cytosolic entry efficiencies of CPP12, BCP16a, BCP16b, and pBCP427-4a were also assessed by confocal microscopy of NIH 3T3 cells after treatment with 2 μM peptide at 37° C. for 2 h. Each peptide resulted in diffuse fluorescence in the cytoplasmic region of the cells, demonstrating that a significant fraction of the internalized peptide reached the cytosol (FIG. 12). Largely consistent with the flow cytometry results, the fluorescence intensities of the treated cells suggest that BCP16a and BCP16b enter the cytosol of 3T3 cells with similar efficiencies to CPP12, whereas pBCP427-4a is less active than CPP12.

TABLE 8
Structures, Cytosolic Entry Efficiency, and Serum Stability of BCPs

| | | | MFI^{NF} | |
|---|---|---|---|---|
| SEQ ID | | | (Cytosolic | Serum |
| NO. | CPP | Sequenceª | Entry)^{b} | t_{1/2} (h) |
| 10 | CPP12 | cyclo(FfΦRrRrQ) | 100 | >24 |
| 11 | Tat | Ac-YGRKKRRQRRR | 5.0 ± 0.6 | <0.5 |
| | BCP4 | Ac-CrRrRCΦfC | 51 ± 16 | 18 |
| 58 | BCP16 | HS-CH₂-CO- | 444 ± 2 | 1.1 |
| | | rRrRCΦ(Bta)φC | | |
| 82 | BCP16a | HS-CH₂-CO- | 156 ± 4 | ~24 |
| | | rRrRcΦ(Bta)φC | | |
| 83 | BCP16b | HS-CH₂-CO- | 98 ± 1 | ~24 |
| | | rRrRCφ(Bta)φC | | |
| 68 | pBCP427-4 | Ac-CWSNWFCFQRRC | 189 ± 6 | 1.6 |
| 84 | pBCP427-4a | Ac-cwsnwfcfqrrc | 33 ± 1 | >24 |
| 85 | pBCP427- | Ac-AWSNWFAFQRRA | 18 ± 2 | 0.56 |
| | 4Ala | | | |

ªAll peptides contained a C-terminal miniPEG-Lys(NF)-NH₂. Bta, 3-(3-benzothienyl)-L-alanine; Φ, L-2-naphthylalanine; φ, D-2-naphthylalanine; lowercase letters, D-amino acids; and NF, naphthofluorescein.
^{b}All values were determined at 2 μM peptide and are relative to that of CPP12 (100%).

BCP16 Analogs of Decreased Positive Charges. Some polycationic CPPs cause cytotoxicity to mammalian cells in vitro and acute toxicity in animals (Aguilera, T. A., et al., (2009). Systemic in vivo distribution of activatable cell penetrating peptides is superior to that of cell penetrating peptides. Integrative biology: quantitative biosciences from nano to macro, 1(5-6), 371-381; Lafarga, V., et al., (2021). Widespread displacement of DNA- and RNA-binding factors underlies toxicity of arginine-rich cell-penetrating peptides. The EMBO journal, 40(13), e103311). While the molecular mechanism of toxicity remains poorly defined, the toxicity appears to be correlated with the number of positive changes especially the number of arginine residues in the CPP. Since BCP16 shows some cytotoxicity to HEK293T and HeLa cells at high concentrations (FIGS. 9A-9B), a limited medicinal chemistry campaign was undertaken to reduce the charge and/or hydrophobicity of BCP16. Thus, in BCP17-21, one or two arginine residues were replaced by citrullines, which have a neutral side chain but maintain some of the hydrogen-bonding capabilities of arginine (Table 9). For BCP22-24, one of the large hydrophobic residues (Nal, Bta, or D-Nal) was replaced by a phenylalanine or tryptophan. The peptides were similarly labeled with NF at the C-terminus and their cytosolic entry efficiencies into HeLa cells were assessed by flow cytometry.

At 2 μM, BCP17 showed 361% cytosolic entry efficiency relative to that of CPP12 (or 81% of BCP16), suggesting it is feasible to reduce the charge of the peptide and maintain a highly efficient BCP (Table 9).

TABLE 9
Structures and Cytosolic Entry Efficiency of Additional BCPs

| | | | MFI^{NF} | No. of |
|---|---|---|---|---|
| SEQ ID | | | (Cytosolic | Replicates |
| NO. | CPP | Sequenceª | Entry)^{b} | (n) |
| 10 | CPP12 | cyclo(FfΦRrRrQ) | 100 | 3 |
| 11 | Tat | Ac-YGRKKRRQRRR | 5.0 ± 0.6 | 3 |
| 58 | BCP16 | HS-CH₂-CO-rRrRCΦ(Bta)φC | 444 ± 2 | 2 |
| 59 | BCP17 | HS-CH₂-CO-r(L-Cit)rRCΦ(Bta)φC | 361 ± 18 | 3 |
| 75 | BCP18 | HS-CH₂-CO-rRr(L-Cit)CΦ(Bta)φC | | |
| 76 | BCP19 | HS-CH₂-CO-(d-cit)RrRCΦ(Bta)φC | | |
| 77 | BCP20 | HS-CH₂-CO-rR(d-cit)RCΦ(Bta)φC | | |
| 78 | BCP21 | HS-CH₂-CO-(d-cit)(L- | | |
| | | Cit)rRCΦ(Bta)φC | | |
| 79 | BCP22 | HS-CH₂-CO-rRrRCΦWφC | | |
| 80 | BCP23 | HS-CH₂-CO-rRrRCF(Bta)φC | | |
| 81 | BCP24 | HS-CH₂-CO-rRrRCΦ(Bta)fC | | |

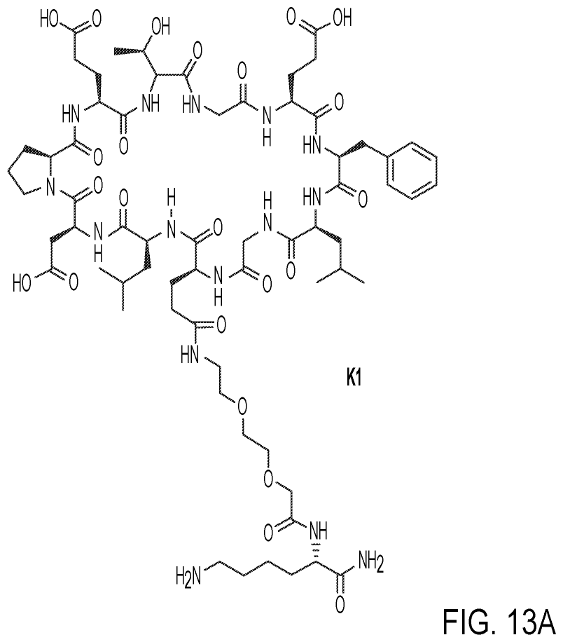

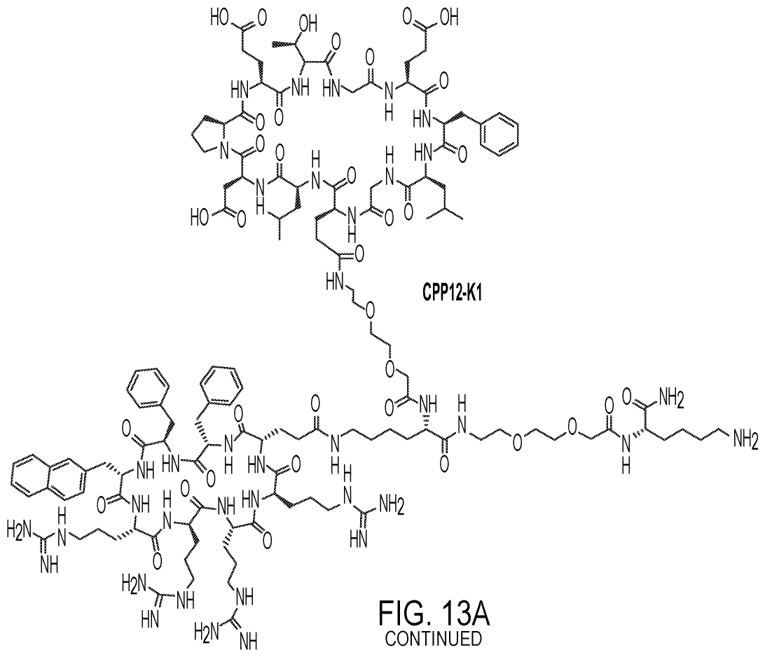

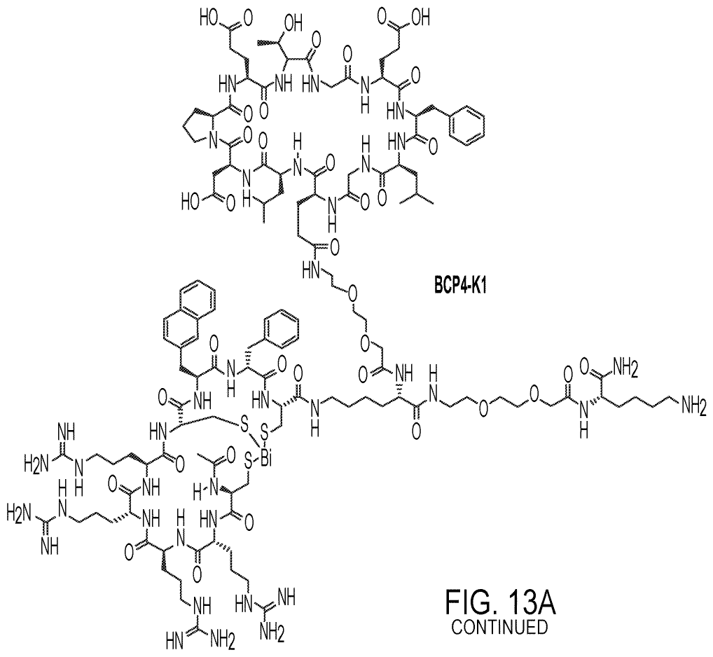

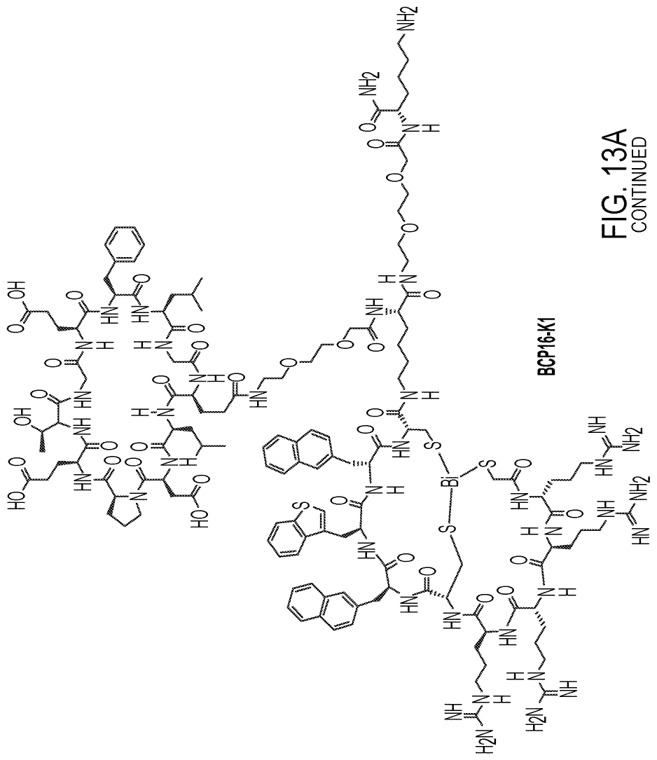

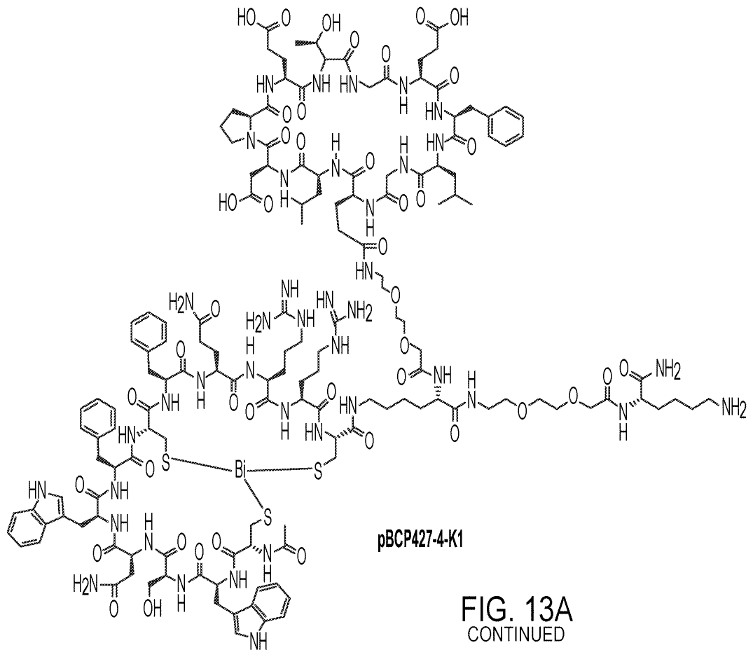

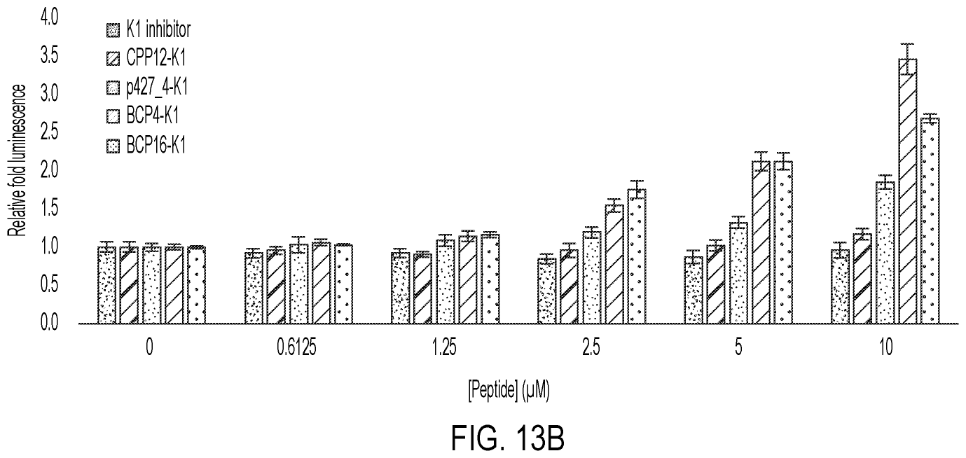

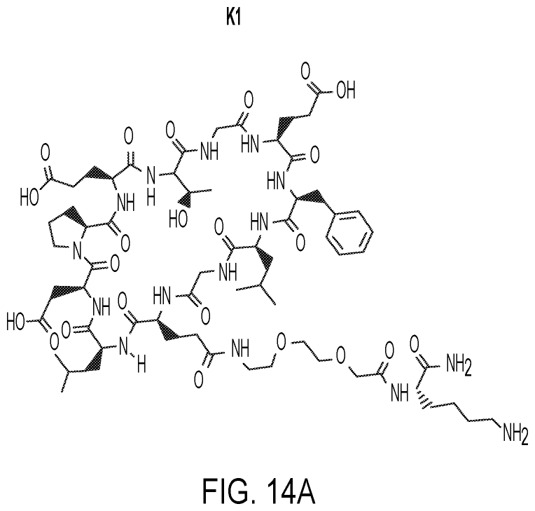

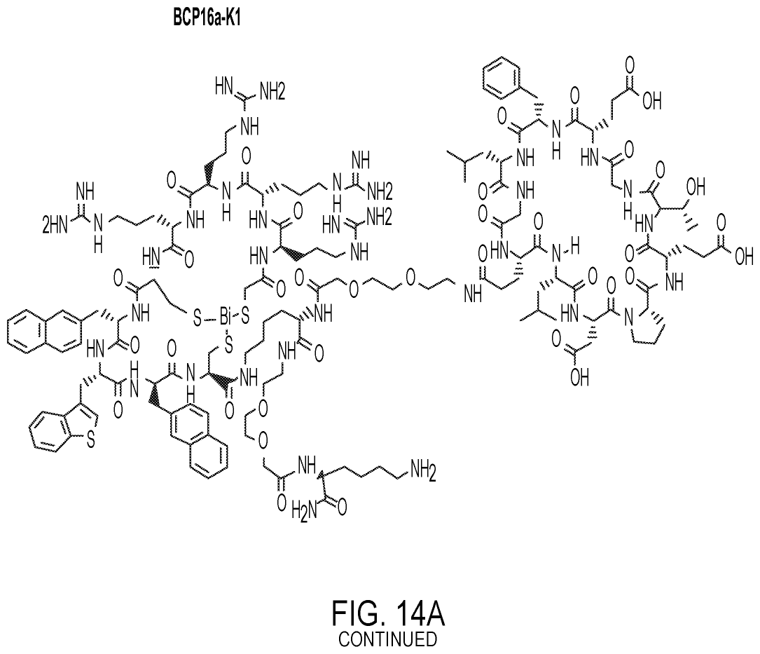

Cytosolic Delivery of Peptidyl Cargo. To assess the ability of BCPs to deliver peptidyl cargo into mammalian cells, the BCPs were covalently conjugated to a cyclic peptidyl inhibitor of the Keap1-Nrf2 interaction, cyclo(GQLDPETGEFL (K1, K_{D}=18 nM for Keap1) (Lu, M. C., et al., (2018). Discovery of a head-to-tail cyclic peptide as the Keap1-Nrf2 protein-protein interaction inhibitor with high cell potency. European journal of medicinal chemistry, 143:1578-1589), to peptides BCP4-K1, BCP16-K1, pBCP427-4-K1 (FIGS. 13A-13B). For comparison, cyclic CPP12 was similarly conjugated to K1 and K1 was synthesized as a negative control. The BCP-K1 and CPP12-K1 conjugates were examined for their inhibition of the intracellular Keap1-Nrf2 interaction using an ARE reporter-HepG2 cell line, which contains a firefly luciferase gene under the transcriptional control of Nrf2 (Lee, J. M., & Johnson, J. A. (2004). An important role of Nrf2-ARE pathway in the cellular defense mechanism. Journal of biochemistry and molecular biology, 37(2), 139-1438). Under basal conditions, Nrf2 interacts with Keap1 and is retained in the cytosol or degraded by the proteasome. However, upon blocking the Keap1-Nrf2 interaction, Nrf2 accumulates and translocates into the nucleus, inducing the expression of luciferase.

One hundred μL of ARE-Reporter Hep G2 cells were seeded in a 96-well plate at 5000 cells/well in minimum essential media supplemented with 10% FBS and 1% penicillin/streptomycin. The following day, 10 μL of peptide of various concentrations was added to the cells to give final concentrations of 0.625-10 μM. Cells treated with DMSO were used as a control. After 18 h, the cells were treated with an equal volume (110 μL) of BPS bioscience ONE-Step luciferase assay solution and shaken for 30 min. The luminescent signal was measured on a Tecan Infinite Pro plate reader.

BCP4-K1, BCP16-K1, and pBCP427-4-K1 induced the luminescence signal of HepG2 cells in dose-dependent manners, with up to 3.5-fold induction at 10 μM peptide concentration, whereas peptide K1 did not (FIG. 13A-13B). These results demonstrate that the BCPs successfully delivered peptide K1 into the cytosol of HepG2 cells and the BCP-K1 conjugates are biologically active inside the cytosol of the cells. However, quantitative comparison of the cytosolic entry efficiencies of the BCPs and CPPs is complicated by several factors. First, they have very different proteolytic stabilities during the 18-h incubation period; while cyclic CPP12 has a serum tin of >24 h, BCP4, BCP16, and pBCP427-4 have serum tin values of 18, 1.1, and ˜2 h, respectively (Table 8). Second, binding of BCPs/CPPs to serum and/or intracellular proteins may significantly inhibit the cellular entry efficiency and Keap1 binding inside the cytosol, respectively. Finally, the positively charged cyclic CPP12 and BCPs may interact intramolecularly with the negatively charged K1 peptide, inhibiting the cellular entry and/or Keap1 binding of the conjugates (Buyanova, M., et al., (2022). Discovery of a Cyclic Cell-Penetrating Peptide with Improved Endosomal Escape and Cytosolic Delivery Efficiency. Molecular pharmaceutics, 19(5), 1378-13884). One or more of the factors may have contributed to the poor performance of cyclic CPP12 in the ARE-Reporter assay (FIGS. 13A-13B).

Cytosolic Entry Efficiency. The cytosolic entry efficiencies of BCP16a, BCP16b, and pBCP427-4a were remeasured. They are 156%, 98%, and 33%, respectively, relative to that of CPP12 (100%). These values should replace those reported earlier. To test the significance of bismuth cyclization, an alanine analog of pBCP427-4 was synthesized yielding pBCP427-4Ala (AWSNWFAFQRRA), which has a cytosolic entry efficiency of 18%. Thus, cyclization by bismuth improved the cytosolic entry efficiency by ˜10-fold (Table 10).

TABLE 10
Structures, Cytosolic Entry Efficiency, and Serum Stability of BCPs

| | | | MFI^{NF} | |
|---|---|---|---|---|
| SEQ ID | | | (Cytosolic | Serum |
| NO. | CPP | Sequenceª | Entry)^{b} | t_{1/2} (h) |
| 10 | CPP12 | cyclo(FfΦRrRrQ) | 100 | >24 |
| 11 | Tat | Ac-YGRKKRRQRRR | 5.0 ± 0.6 | <0.5 |
| 33 | BCP4 | Ac-CrRrRCΦfC | 51 ± 16 | 18 |
| 58 | BCP16 | HS-CH₂-CO- | 444 ± 2 | 1.1 |
| | | rRrRCΦ(Bta)φC | | |
| 82 | BCP16a | HS-CH₂-CO- | 156 ± 4 | ~24 |
| | | rRrRcΦ(Bta)φC | | |
| 83 | BCP16b | HS-CH₂-CO- | 98 ± 1 | ~24 |
| | | rRrRCφ(Bta)φC | | |
| 68 | pBCP427-4 | Ac-CWSNWFCFQRRC | 189 ± 6 | 1.6 |
| 84 | pBCP427-4a | Ac-cwsnwfcfqrrc | 33 ± 1 | >24 |
| 85 | pBCP427- | Ac-AWSNWFAFQRRA | 18 ± 2 | 0.56 |
| | 4Ala | | | |

ªAll peptides contained a C-terminal miniPEG-Lys(NF)-NH₂. Bta, 3-(3-benzothienyl)-L-alanine; Φ, L-2-naphthylalanine; φ, D-2-naphthylalanine; lowercase letters, D-amino acids; and NF, naphthofluorescein.
^{b}All values were determined at 2 μM peptide and are relative to that of CPP12 (100%).

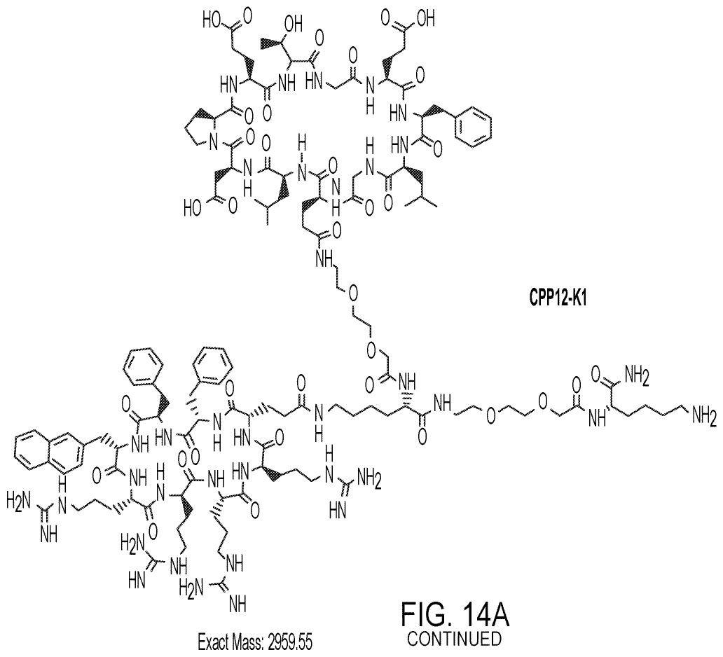

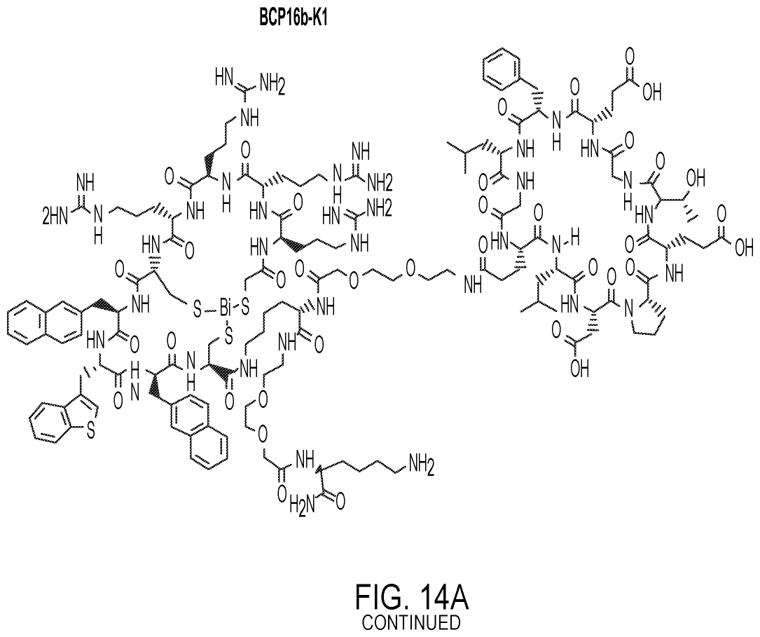

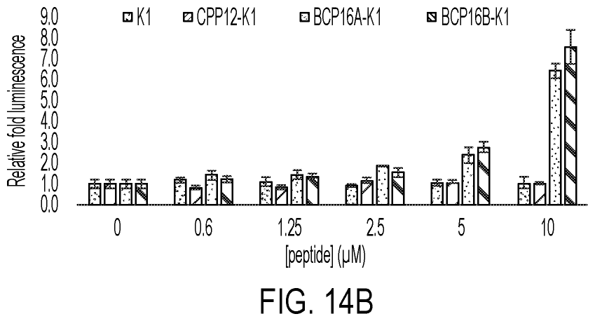

Cytosolic Delivery of Keap1 Inhibitors by BCP16a and BCP16b. BCP16a and BCP16b were covalently conjugated to Keap1 inhibitor K1, cyclo(GQLDPETGEFL (K_{D}=18 nM for Keap1), to produce peptides BCP16a-K1 and BCP16b-K1 (FIG. 14A). K1 and CPP12-K1 were used for comparison and the assay was performed as described above. BCP16a-K1 and BCP16b-K1 induced the luminescence signal of HepG2 cells in dose-dependent manners, with up to 7-fold induction at 10 μM peptide concentration, whereas peptide K1 did not (FIG. 14B). These results demonstrate that the BCP16a and BCP16b successfully delivered peptide K1 into the cytosol of HepG2 cells and the BCP-K1 conjugates are biologically active inside the cytosol of the cells. CPP12-K1 failed to show significant activity in this assay, likely because of intramolecular interaction/interference between the positively charged CPP12 and the negatively charged K1.

Intracellular Delivery of Protein by pBCP427-4. To test whether BCPs can deliver protein cargos into the cell, we fused pBCP427-4 to the N-terminus of superecliptic-pHluorin (SEP), a pH-sensitive GFP variant (1), through the recombinant DNA technology. Specifically, DNA primers 5′-taattggttttgttttcaacgtcgttgtGGAAGTTCAGGCAGTAGCAAAGG-3′ (SEQ ID NO:86) and 5′-gaccaacaagaagaaccagaagaacccatGGGTTTGTGCCCACATGG-3′ (SEQ ID NO:87) were chemically synthesized and used to amplify the plasmid DNA coding for SEP, pET-22b(+)-SEP. This cloning procedure resulted in the insertion of peptide sequence MGSSGSSCWSNWFCFQRRC (SEQ ID NO:88) into the N-terminus of SEP. The resulting fusion protein, pBCP427-4-SEP, has the following amino-acid sequence (with the BCP sequence underlined).

(SEQ ID NO: 89)
MPCGHKPMGSSGSSCWSNWFCFQRRCGSSGSSKGEELFTGVVPILVELD
GDVNGHKFSVSGEGEGDATYGKLTLKFICTTGKLPVPWPTLVTTLTYGV
QCFSRYPDHMKRHDFFKSAMPEGYVQERTIFFKDDGNYKTRAEVKFEGD
TLVNRIELKGIDFKEDGNILGHKLEYNYNDHQVYIMADKQKNGIKANFK
IRHNIEDGGVQLADHYQQNTPIGDGPVLLPDNHYLFTTSTLSKDPNEKR
DHMVLLEFVTAAGITHGMDELYKLEHHHHHH

Escherichia coli BL21 (DE3) Rosetta cells were transformed with the plasmid encoding pBCP427-4-SEP and grown in Luria broth supplemented with 75 mg/L ampicillin to an OD₆₀₀ of 0.6. Protein expression was induced by the addition of 0.25 mM (final concentration) IPTG and 0 5 mM colloidal bismuth subcitrate (CBS) for 5 h at 37° C. The cells were pelleted by centrifugation and suspended in 50 mL (for 1 L of cell culture) of Tris buffer (25 mM Tris, pH 7.0, 150 mM NaCl) supplemented with 0.2 mg/mL lysozyme and a protease inhibitor cocktail (Sigma). After incubation at 4° C. for 30 min, the cell lysate was briefly sonicated and clarified by centrifugation at 15,000 g for 20 min. The crude protein was purified with a HisTrap Excel column on an AKTA GO FLPC, eluted with a linear gradient of 10-500 mM imidazole in the above Tris buffer. The protein was concentrated and exchanged into a low salt pH 7.0 Tris buffer before flash frozen and stored at −80° C.

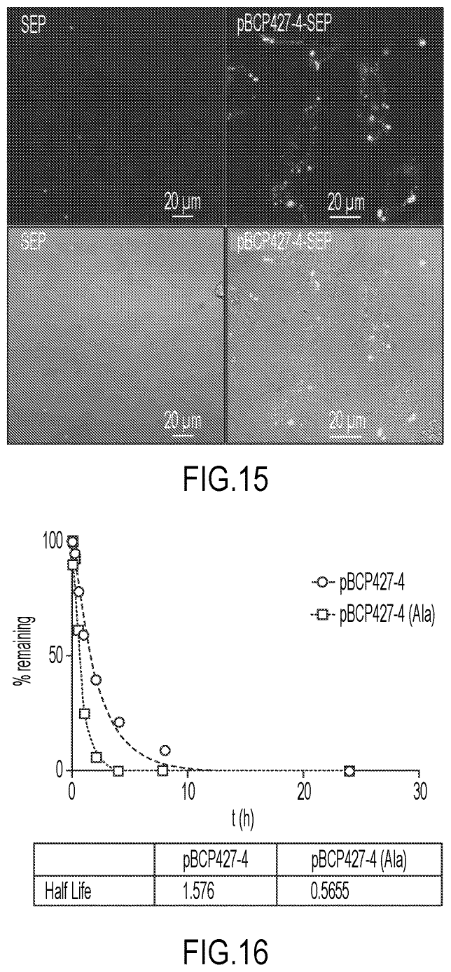

To assess cellular delivery by confocal microscopy, HeLa cells were cultured overnight in a 4-well, 35-mm glass bottom microscope dish at a density of 50,000 cells/mL (300 μL per well). The following day, the cells were washed twice with DPBS and incubated with 5 μM protein for 4 h in phenol red free DMEM with 1% FBS. The cells were washed twice with fresh phenol red free DMEM and incubated with 5 μg/mL Hoechst 33342 for 15 min to stain the nucleus. The cells were washed again with fresh media and imaged with a Nikon A1R confocal microscope. HeLa cells treated with pBCP427-4-SEP showed intense GFP fluorescence inside the cell, although the fluorescence was predominantly punctate, whereas the cells treated with SEP showed no visible fluorescence under the same conditions (FIG. 15). These results indicate that pBCP427-4-SEP entered the HeLa cells but a significant fraction of the internalized proteins remained entrapped inside the endosome/lysosome.

Proteolytic Stability of pBCP427-4 and pBCP427-4Ala. BCPs (100 μM) were incubated in 25% human serum diluted in phosphate-buffered saline for 24 h at 37° C. One hundred-μL aliquots were withdrawn from the mixture at various time points and mixed with 200 μL of 1:1 (v/v) 15% trichloroacetic acid in methanol and acetonitrile. The samples were centrifuged for 5 min in a microcentrifuge and stored at 4° C. until analysis by UPLC-MS equipped with a C-18 column. The column was eluted with a linear gradient of 5-100% acetonitrile in water containing 0.1% TFA over 7.5 min and the eluant was monitored at 214 nm. The area underneath the peak for any remaining BCP was integrated and compared to that at time 0 and the percentage of remaining BCP was plotted as a function of the incubation time. pBCP427-4 and pBCP427-4Ala were degraded in human serum with t_{1/2} values of 1.6 and 0.56 h, respectively (FIG. 16).

1. A peptide, comprising: a cell-penetrating peptide domain of from about 7 to about 25 amino acids in length; wherein the cell-penetrating peptide domain comprises any combination of at least two arginines or arginine analogs and at least two amino acids having a hydrophobic side chain selected from an aryl or heteroaryl, wherein the aryl and heteroaryl are optionally substituted, and wherein the cell penetrating peptide domain comprises at least three thiol containing residues and which are separated from one another by at least one amino acid.

2. The peptide of claim 1, wherein the cell penetrating peptide domain is from about 8 to about to about 14 amino acids amino acids in length.

3. The peptide of claim 1, wherein one or more of the thiol containing residues are cysteine or cysteine analog.

4. The peptide of claim 1, wherein one or more of the amino acids in the cell penetrating peptide domain are non-natural amino acids.

5. The peptide of claim 1, wherein one or more of the amino acids in the cell penetrating peptide domain are D-amino acids.

6. The peptide of claim 1, wherein at least one of the hydrophobic amino acids has an aryl side chain.

7. The peptide of claim 1, further comprising bismuth.

8. The peptide of claim 7, wherein the cell penetrating domain is represented by the following structure: 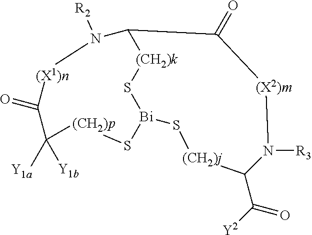
wherein,
X¹ and X² are amino acids;
n and m are independently selected from 1 to 20;
j and k are independently selected from 1 to 4;
p is selected from 0 to 10;
Y^{1a} and Y^{1b} are independently selected from H, OH, when Y^{1b} is not OH or NR¹Y¹, NR¹Y¹, when Y^{1b} is not OH or NR¹Y¹, C₁-C₆alkyl, arylC₀-C₆alkyl, heteroarylC₀-C₆alkyl where aryl and heteroaryl are optionally substituted;
Y¹ is H, a protecting group, a counterion, a cargo moiety joined by an optional linker, or a third peptide domain with an optional linker moiety;
R¹, R², and R³ are independently selected from H, C₁-C₆alkyl, arylC₁-C₆alkyl, heteroarylC₁-C₆alkyl where aryl and heteroaryl are optionally substituted;
Y² is OH, NH₂, a protecting group of the carboxylate, a counterion of the carboxylate, a cargo moiety joined by an optional linker, or a third peptide domain with an optional linker moiety;
wherein, the methylene group in (CH₂)p, (CH₂)k or (CH₂)j is optionally substituted with C₁-C₆alkyl, or linked to R¹, R² or R³ to form a ring; and
wherein at least two arginines or arginine-analogs and at least two amino acids having a hydrophobic side chain selected from an aryl or heteroaryl, wherein the aryl and heteroaryl are optionally substituted, are present among (X¹)n and (X²)m.

9. The peptide of claim 8, wherein the at least two arginines or arginine-analogs are in (X¹)n or (X²)m.

10. The peptide of claim 8, wherein the at least two arginines or arginine-analogs are distributed among (X¹)n and (X²)m.

11. The peptide of claim 8, wherein the at least two amino acids having hydrophobic side chains are in (X¹)n or (X²)m.

12. The peptide of claim 8, wherein the at least two amino acids having hydrophobic side chains are distributed among (X¹)n and (X²)m.

13. The peptide of claim 8, wherein the at least two or three amino acids having hydrophobic side chains selected from an aryl or heteroaryl, wherein the aryl and heteroaryl are optionally substituted are distributed among the two domains.

14. The peptide of claim 8, wherein the peptide domains comprise at least three, at least four, at least five, at least six, or at least seven arginines.

15. The peptide of claim 8, wherein the peptide domains comprise four arginines.

16. The peptide of claim 8, wherein either (X¹)n or (X²)m comprises at least two or at least three adjacent amino acids having hydrophobic side chains selected from an aryl or heteroaryl, wherein the aryl and heteroaryl are optionally substituted, and the other (X¹)n or (X²)m comprises at least three, at least four, at least five, at least six, or at least seven adjacent arginines or arginine analogs.

17. The peptide of claim 8, wherein either (X¹)n or (X²)m comprises two adjacent amino acids having hydrophobic side chains selected from fused aryl, fused heteroaryl, or non-aromatic polycyclic cycloalkyl radicals.

18. The peptide of claim 8, wherein the arginines or arginine-analogs are adjacent to one another or distributed throughout the CPP.

19. The peptide of claim 8, wherein Y^{1a}, Y^{1b}, Y¹, or Y² comprises a cargo moiety.

20. A peptide having SEQ ID NO. 12-85.

21. A fusion protein comprising a therapeutic protein domain conjugated to the peptide of any claim 20.

22. The fusion protein of claim 21, wherein the therapeutic domain is conjugated though a linker.

23. A method of cyclizing a peptide comprising contacting a peptide of any one of claim 20 with a bismuth salt.
